(19) United States
(12) Patent Application Publication
(10) Pub. No.: US 2016/0135737 A1
(43) Pub. Date: May 19, 2016

(54) DETERMINING TREATMENT COMPLIANCE USING SPEECH PATTERNS CAPTURED DURING USE OF A COMMUNICATION SYSTEM

(71) Applicant: Elwha LLC, Bellevue, WA (US)

(72) Inventors: Jeffrey A. Bowers, Bellevue, WA (US); Paul Duesterhoft, Grapevine, TX (US); Daniel Hawkins, Pleasanton, CA (US); Roderick A. Hyde, Redmond, WA (US); Edward K.Y. Jung, Bellevue, WA (US); Jordin T. Kare, San Jose, CA (US); Eric C. Leuthardt, St. Louis, MO (US); Nathan P. Myhrvold, Medina, WA (US); Michael A. Smith, Phoenix, AZ (US); Elizabeth A. Sweeney, Seattle, WA (US); Clarence T. Tegreene, Mercer Island, WA (US); Lowell L. Wood, JR., Bellevue, WA (US)

(21) Appl. No.: 14/729,322

(22) Filed: Jun. 3, 2015

Related U.S. Application Data
(63) Continuation-in-part of application No. 14/543,030, filed on Nov. 17, 2014.
Application No. 14/543,030 is a continuation-in-part of application No. 14/543,066, filed on Nov. 17, 2014.

(51) Int. Cl. A61B 5/00; G10L 17/00; G06F 19/00

(52) U.S. Cl. CPC A61B 5/4833; A61B 5/4803; G06F 19/3487; G10L 17/00; G06F 19/36

(57) ABSTRACT

Methods and systems for monitoring compliance of a patient with a prescribed treatment regimen are described. Compliance is determined based upon analysis of the patient's speech detected during use of a communication system such as a mobile telephone by the patient. Methods and systems as described herein can be used for monitoring patient compliance with a treatment for a brain-related disorder, for example. Identity of the patient as user of the communication system is determined through the use of e.g., biometric or authentication techniques. Speech data indicative of whether the patient has complied with the prescribed treatment regimen is transmitted from a circuitry-based system at the patient location to a monitoring location, where it may be reviewed by a medical caregiver or other party, for example. Patient speech may be analyzed at the patient location and/or subjected to analysis at the monitoring location.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority Application(s)).

### PRIORITY APPLICATIONS

The present application constitutes a continuation-in-part of U.S. patent application Ser. No. 14/543,030, entitled MONITORING TREATMENT COMPLIANCE USING SPEECH PATTERNS PASSIVELY CAPTURED FROM A PATIENT ENVIRONMENT, naming Jeffrey A. Bowers, Paul Duesterhoft, Daniel Hawkins, Roderick A. Hyde, Edward K. Y. Jung, Jordin T. Kare, Eric C. Leuthardt, Nathan P. Myhrvold, Michael A. Smith, Elizabeth A. Sweeney, Clarence T. Tegreene, and Lowell L. Wood, Jr. as inventors, filed 17 Nov. 2014 with attorney docket no. 0810-004-006-000000, which is currently co-pending or is an application of which a currently co-pending application is entitled to the benefit of the filing date.
The present application constitutes a continuation-in-part of U.S. patent application Ser. No. 14/543,066, entitled DETERMINING TREATMENT COMPLIANCE USING SPEECH PATTERNS PASSIVELY CAPTURED FROM A PATIENT ENVIRONMENT, naming Jeffrey A. Bowers, Paul Duesterhoft, Daniel Hawkins, Roderick A. Hyde, Edward K. Y. Jung, Jordin T. Kare, Eric C. Leuthardt, Nathan P. Myhrvold, Michael A. Smith, Elizabeth A. Sweeney, Clarence T. Tegreene, and Lowell L. Wood, Jr. as inventors, filed 17 Nov. 2014 with attorney docket no. 0810-004-007-000000, which is currently co-pending or is an application of which a currently co-pending application is entitled to the benefit of the filing date.

If the listings of applications provided above are inconsistent with the listings provided via an ADS, it is the intent of the Applicant to claim priority to each application that appears in the Domestic Benefit/National Stage Information section of the ADS and to each application that appears in the Priority Applications section of this application.

All subject matter of the Priority Applications and of any and all applications related to the Priority Applications by priority claims (directly or indirectly), including any priority claims made and subject matter incorporated by reference therein as of the filing date of the instant application, is incorporated herein by reference to the extent such subject matter is not inconsistent herewith.

### SUMMARY

In an aspect, a system includes, but is not limited to, at least one receiving device for use at a monitoring location for receiving a speech data signal transmitted to the monitoring location from a patient location, the speech data signal containing speech data, the speech data representing at least one speech pattern in speech sensed from a patient with at least one audio sensor in a communication system at the patient location during use of the communication system by the patient, and the patient having a brain-related disorder and a prescribed treatment regimen for treating at least one aspect of the brain-related disorder; patient identification circuitry configured to determine a presence of the patient from at least one identity signal received at the monitoring location from the patient location; signal processing circuitry configured to analyze the speech data signal to determine whether the speech data is representative of at least one speech pattern that matches at least one characteristic speech pattern; compliance determination circuitry configured to determine whether the patient has complied with the prescribed treatment regimen based upon whether the speech data represents the at least one speech pattern that matches the at least one characteristic speech pattern; and reporting circuitry configured to report a conclusion based on the determination of whether the patient has complied with the prescribed treatment regimen. In addition to the foregoing, other system aspects are described in the claims, drawings, and text forming a part of the disclosure set forth herein.

In an aspect, a method of monitoring compliance of a patient with a prescribed treatment regimen includes, but is not limited to, receiving a speech data signal with a receiving device at a monitoring location, the speech data signal transmitted to the monitoring location from a patient location, the speech data signal containing speech data representing at least one speech pattern in speech sensed from a patient by at least one audio sensor of a communication system at the patient location during use of the communication system by the patient, the patient having a brain-related disorder and a prescribed treatment regimen for treating at least one aspect of the brain-related disorder; determining a presence of the patient with patient identification circuitry at the monitoring location from at least one identity signal received at the monitoring location from the patient location; analyzing the speech data signal with signal processing circuitry at the monitoring location to determine whether the speech data represents at least one speech pattern that matches at least one characteristic speech pattern; determining with compliance determination circuitry whether the patient has complied with the prescribed treatment regimen based on whether the speech data represents the at least one speech pattern that matches the at least one characteristic speech pattern; and reporting with reporting circuitry a conclusion based on the determination of whether the patient has complied with the prescribed treatment regimen. In addition to the foregoing, other method aspects are described in the claims, drawings, and text forming a part of the disclosure set forth herein.

In an aspect, a computer program product includes, but is not limited to, a non-transitory signal-bearing medium bearing one or more instructions for receiving a speech data signal with a receiving device at a monitoring location, the speech data signal transmitted to the monitoring location from a patient location, the speech data signal containing speech data representing at least one speech pattern in speech sensed from a patient by at least one audio sensor of a communication system at the patient location during use of the communication system by the patient, the patient having a brain-related disorder and a prescribed treatment regimen for treating at least one aspect of the brain-related disorder; one or more instructions for determining a presence of the patient with patient identification circuitry at the monitoring location from at least one identity signal received at the monitoring location from the patient location; one or more instructions for analyzing the speech data signal with signal processing circuitry at the monitoring location to determine whether the speech data represents at least one speech pattern that matches at least one characteristic speech pattern; one or more instructions for determining with compliance determination circuitry whether the patient has complied with the prescribed treatment regimen based on whether the speech data represents the at least one speech pattern that matches the at least one characteristic speech pattern; and one or more instructions for reporting with reporting circuitry a conclusion based on the determination of whether the patient has complied with the prescribed treatment regimen. In addition to the foregoing, other aspects of a computer program product including one or more non-transitory machine-readable data storage media bearing one or more instructions are described in the claims, drawings, and text forming a part of the disclosure set forth herein.

In an aspect, a system includes, but is not limited to, a computing device, and instructions that when executed on the computing device cause the computing device to receive a speech data signal with a receiving device at a monitoring location, the speech data signal transmitted to the monitoring location from a patient location, the speech data signal containing speech data representing at least one speech pattern in speech sensed from a patient by at least one audio sensor of a communication system at the patient location during use of the communication system by the patient, the patient having a brain-related disorder and a prescribed treatment regimen for treating at least one aspect of the brain-related disorder; determine a presence of the patient with patient identification circuitry at the monitoring location from at least one identity signal received at the monitoring location from the patient location; analyze the speech data signal with signal processing circuitry at the monitoring location to determine whether the speech data represents at least one speech pattern that matches at least one characteristic speech pattern; determine with compliance determination circuitry whether the patient has complied with the prescribed treatment regimen based on whether the speech data represents the at least one speech pattern that matches the at least one characteristic speech pattern; and report with reporting circuitry a conclusion based on the determination of whether the patient has complied with the prescribed treatment regimen. In addition to the foregoing, other aspects of a computing device are described in the claims, drawings, and text forming a part of the disclosure set forth herein.

In an aspect, a communication system includes, but is not limited to, at least one audio sensor for sensing at least one audio signal including patient speech during use of the communication system by a patient at a patient location, the patient having a brain-related disorder and a prescribed treatment regimen for treating at least one aspect of the brain-related disorder; patient identification circuitry configured to determine a presence of the patient from at least one identity signal sensed at the patient location; speech detection circuitry in the communication system for processing the at least one audio signal to identify at least one section of the at least one audio signal containing speech of the patient, wherein the speech detection circuitry is configured to identify the at least one section of the at least one audio signal containing speech of the patient based at least in part on the determination of the presence of the patient by the patient identification circuitry; speech analysis circuitry in the communication system for analyzing the at least one section of the at least one audio signal to generate speech data including data indicative of whether the patient has complied with the prescribed treatment regimen; and at least one transmitting device at the patient location for transmitting a speech data signal containing the speech data including data indicative of whether the patient has complied with the prescribed treatment regimen from the patient location to a receiving device at a monitoring location. In addition to the foregoing, other system aspects are described in the claims, drawings, and text forming a part of the disclosure set forth herein.

In an aspect, a method of monitoring compliance of a patient with a prescribed treatment regimen includes, but is not limited to, sensing at least one audio signal including patient speech from a patient with at least one audio sensor of a communication system at a patient location during use of the communication system by the patient, the patient having a brain-related disorder and a prescribed treatment regimen for treating at least one aspect of the brain-related disorder; determining a presence of the patient with patient identification circuitry from at least one identity signal sensed at the patient location; processing the at least one audio signal with a speech detection circuitry in the communication system to identify at least one section of the at least one audio signal containing speech of the patient, including identifying speech from the patient based at least in part on the determination of the presence of the patient by the patient identification circuitry; analyzing the at least one section of the at least one audio signal with speech analysis circuitry in the communication system to generate speech data including data indicative of whether the patient has complied with the prescribed treatment regimen; and transmitting the speech data including data indicative of whether the patient has complied with the prescribed treatment regimen to a receiving device at a monitoring location with at least one transmitting device at the patient location.

In an aspect, a computer program product includes, but is not limited to, a non-transitory signal-bearing medium bearing one or more instructions for sensing at least one audio signal including patient speech from a patient with at least one audio sensor of a communication system at a patient location during use of the communication system by the patient, the patient having a brain-related disorder and a prescribed treatment regimen for treating at least one aspect of the brain-related disorder; one or more instructions for determining a presence of the patient with patient identification circuitry from at least one identity signal sensed at the patient location; one or more instructions for processing the at least one audio signal with a speech detection circuitry in the communication system to identify at least one section of the at least one audio signal containing speech of a patient, including identifying speech from the patient based at least in part on the determination of the presence of the patient by the patient identification circuitry; one or more instructions for analyzing the at least one section of the at least one audio signal with speech analysis circuitry in the communication system to generate speech data including data indicative of whether the patient has complied with the prescribed treatment regimen; and one or more instructions for transmitting the speech data including data indicative of whether the patient has complied with the prescribed treatment regimen to a receiving device at a monitoring location with at least one transmitting device at the patient location. In addition to the foregoing, other aspects of a computer program product including one or more non-transitory machine-readable data storage media bearing one or more instructions are described in the claims, drawings, and text forming a part of the disclosure set forth herein.

In an aspect, a system includes, but is not limited to, a computing device and instructions that when executed on the computing device cause the computing device to sense at least one audio signal including patient speech from a patient with at least one audio sensor of a communication system at a patient location during use of the communication system by the patient, the patient having a brain-related disorder and a prescribed treatment regimen for treating at least one aspect of the brain-related disorder; determine a presence of the patient with patient identification circuitry from at least one identity signal sensed at the patient location; process the at least one audio signal with a speech detection circuitry in the communication system to identify at least one section of the at least one audio signal containing speech of a patient, including identifying speech from the patient based at least in part on the determination of the presence of the patient by the patient identification circuitry; analyze the at least one section of the at least one audio signal with speech analysis circuitry in the communication system to generate speech data including data indicative of whether the patient has complied with the prescribed treatment regimen; and transmit the speech data including data indicative of whether the patient has complied with the prescribed treatment regimen to a receiving device at a monitoring location with at least one transmitting device at the patient location. In addition to the foregoing, other aspects of a computing device are described in the claims, drawings, and text forming a part of the disclosure set forth herein.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a block diagram of a system for monitoring compliance of a patient with a prescribed treatment regimen.

FIG. 2 is a block diagram of an embodiment of a system for monitoring compliance of a patient with a prescribed treatment regimen.

FIG. 3 is a block diagram of components of a system for monitoring compliance of a patient with a prescribed treatment regimen at a patient location.

FIG. 4 is a block diagram of an embodiment of a system for monitoring compliance of a patient with a prescribed treatment regimen.

FIG. 5 is a block diagram of components of a system for monitoring compliance of a patient with a prescribed treatment regimen at a monitoring location.

FIG. 6 illustrates an example embodiment of a thin computing device in which embodiments may be implemented.

FIG. 7 illustrates an example embodiment of a computing system in which embodiments may be implemented.

FIG. 8 illustrates an embodiment of a system for monitoring compliance of a patient with a prescribed treatment regimen.

FIG. 9 illustrates an embodiment of a system for monitoring compliance of a patient with a prescribed treatment regimen.

FIG. 10 is a flow diagram of a method of monitoring compliance of a patient with a prescribed treatment regimen.

FIG. 11 is a flow diagram of further aspects of the method of FIG. 10.

FIG. 12 is a flow diagram of further aspects of the method of FIG. 10.

FIG. 13 is a flow diagram of further aspects of the method of FIG. 10.

FIG. 14 is a flow diagram of further aspects of the method of FIG. 10.

FIG. 15 is a flow diagram of further aspects of the method of FIG. 10.

FIG. 16 is a flow diagram of further aspects of the method of FIG. 10.

FIG. 17 is a flow diagram of further aspects of the method of FIG. 10.

FIG. 18 is a flow diagram of further aspects of the method of FIG. 10.

FIG. 19 is a flow diagram of further aspects of the method of FIG. 10.

FIG. 20 is a flow diagram of further aspects of the method of FIG. 10.

FIG. 21 is a flow diagram of further aspects of the method of FIG. 10.

FIG. 22 is a flow diagram of further aspects of the method of FIG. 10.

FIG. 23 is a block diagram of a computer program product including a signal-bearing medium.

FIG. 24 is a block diagram of a system including a computing device.

FIG. 25 is a flow diagram of a method of monitoring compliance of a patient with a prescribed treatment regimen.

FIG. 26 is a flow diagram of further aspects of the method of FIG. 25.

FIG. 27 is a flow diagram of further aspects of the method of FIG. 25.

FIG. 28 is a flow diagram of further aspects of the method of FIG. 25.

FIG. 29 is a flow diagram of further aspects of the method of FIG. 25.

FIG. 30 is a flow diagram of further aspects of the method of FIG. 25.

FIG. 31 is a flow diagram of further aspects of the method of FIG. 25.

FIG. 32 is a flow diagram of further aspects of the method of FIG. 25.

FIG. 33 is a flow diagram of further aspects of the method of FIG. 25.

FIG. 34 is a block diagram of a computer program product including a signal-bearing medium.

FIG. 35 is a block diagram of a system including a computing device.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

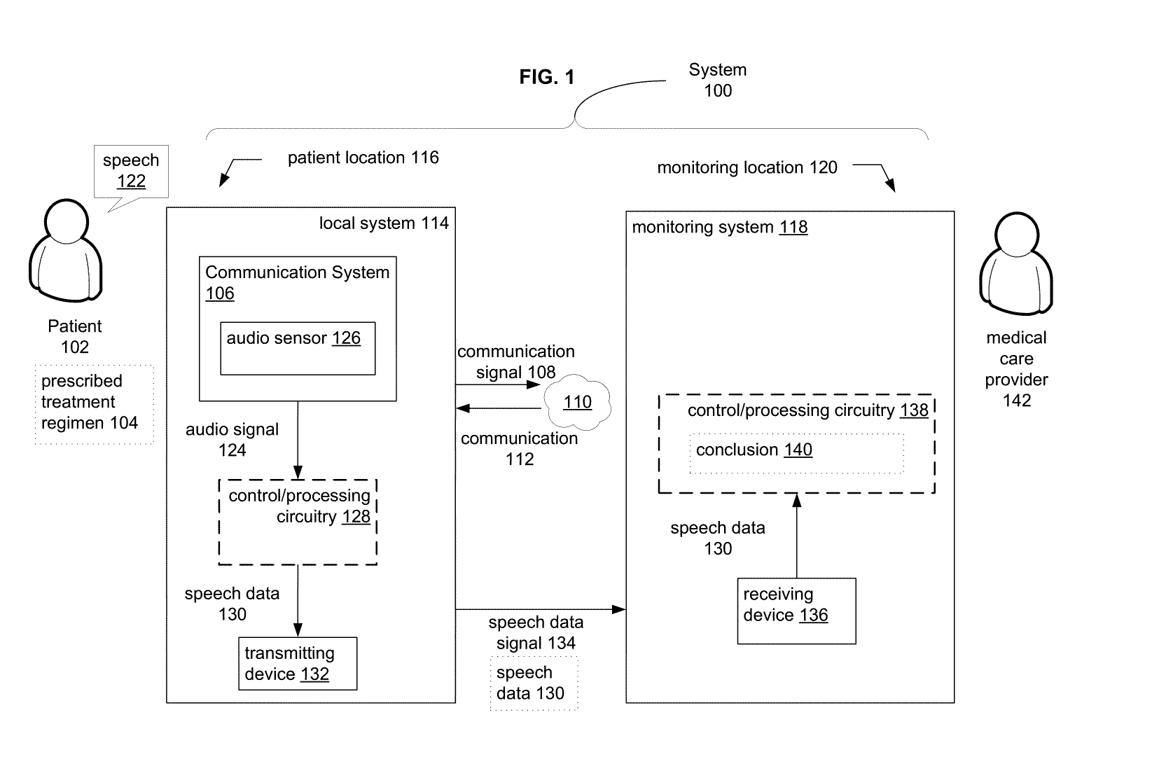

FIG. 1 illustrates a system 100 for monitoring compliance of a patient 102 with a prescribed treatment regimen 104 during use of a communication system 106. Communication system 106 may be, for example and without limitation, a cell phone or a computing system configured to receive voice communications from patient 102 and transmit them, in the form of communication signal 108, to a destination 110. In an aspect, communication system 106 also delivers communication 112 to patient 102. System 100 includes local system 114 at patient location 116 and monitoring system 118 at monitoring location 120. Patient location 116 includes any location at which patient 102 uses communication system 106. In FIG. 1, and in other figures herein, in general, unless context dictates otherwise, solid lines are used to indicate standard components or steps, and dashed lines are used to represent optional components or steps. Unless context indicates otherwise, dotted lines are used to indicate data or information. Dashed lines may also be used to indicate signals.

System 100 monitors compliance of patient 102 with prescribed treatment regimen 104 by detecting and analyzing speech 122 from patient 102 during use of communication system 106. In an aspect, speech 122 (in the form of audio signal 124 sensed by audio sensor 126) is processed by control/processing circuitry 128 in local system 114 to obtain speech data 130. Transmitting device 132 transmits speech data signal 134, including speech data 130, to receiving device 136 at monitoring location 120. Speech data 130 from speech data signal 134 is processed by control/processing circuitry 138 in monitoring system 118 to generate a conclusion 140 (e.g., regarding patient's compliance or lack thereof), which is reported to medical care provider 142. In different embodiments, examples of which are described elsewhere here, different levels of signal processing take place in local system 114 versus monitoring system 118. The location at which different signal processing aspects are performed may depend on availability of data storage space; speed, reliability and/or power consumption of data transmission between patient location 116 and monitoring location 120; and privacy concerns relating to storage and transmittal of patient data, among other considerations. As will be discussed in greater detail herein below, speech data signal 134 may contain raw speech data, information obtained from processed speech data, or both.

In various aspects, speech data signal 134, which is transmitted to monitoring location 120, is distinct from communication signal 108, which is used to carry voice data in connection with the use of communication system 106 for voice communication. For example, if communication system 106 is a cell phone, communication signal 108 is a cell phone signal that is transmitted, via a cellular network, to a destination 110 such as a cell phone used by a party the patient 102 is calling. While patient 102 is engaged in use of communication system 106 for voice communications, communication signal 108 carrying voice communications is transmitted to destination 110, and in addition speech data signal 134 is transmitted to monitoring location 120. In some cases, destination 110 may be at the same location as the monitoring location (e.g., if patient 102 is engaged in a phone call with medical care provider 142) and potentially (but not necessarily) the same device as monitoring system 118, but in other aspects destination 110 is a device at any location on a communication network that is accessible with communication system 106.

In an aspect, patient 102 has a brain-related disorder, and prescribed treatment regimen 104 is a treatment regimen prescribed to patient 102 for treating at least one aspect of the brain-related disorder. Brain-related disorders include, for example, mental disorders, psychological disorders, psychiatric disorder, traumatic disorders, lesion-related disorders, and/or neurological disorders, as discussed in greater detail herein below. Prescribed treatment regimen 104 may include a prescription for one or more therapeutic treatments, including medications, pharmaceuticals, nutraceuticals, therapeutic activities, diet, sleep, exercise, counseling, etc., to be used individually or in combination. In various aspects, prescribed treatment regimen 104 specifies type, quantity, and time course of any or all such therapeutic treatments.

Monitoring system 118 at monitoring location 120 allows medical care provider 142 to remotely monitor compliance of patient 102 with prescribed treatment regimen 104. Monitoring location 120 may be, for example, a hospital, clinic, data center, or doctor's office. Monitoring location 120 may be a short distance away from patient location 116 (e.g., in another room of the same building, or even within the same room as patient location 116) or it may be in a separate building, a few miles away, or many miles away. Monitoring system 118 includes at least one receiving device 136 for use at monitoring location 120 for receiving speech data signal 134 transmitted to monitoring location 120 from patient location 116.

Systems as described herein can be used, for example, to monitor patient compliance with prescribed treatment regimen 104 at the request of or with the cooperation and/or authorization of patient 102, e.g., in the situation that the patient and/or the patient's caregiver wish to track the patient's compliance with the prescribed treatment regimen. In some cases, monitoring of patient compliance with a prescribed treatment regimen can be implemented at the request or requirement of a caregiver, insurance company, or other individual or entity, for example, as a condition of living in a group home, mental health care facility, or other institution. In some cases, monitoring of compliance can be implemented without knowledge and/or authorization of the patient, e.g., in situations in which the patient is not capable of making decisions for his or her self or to fulfill a legal requirement.

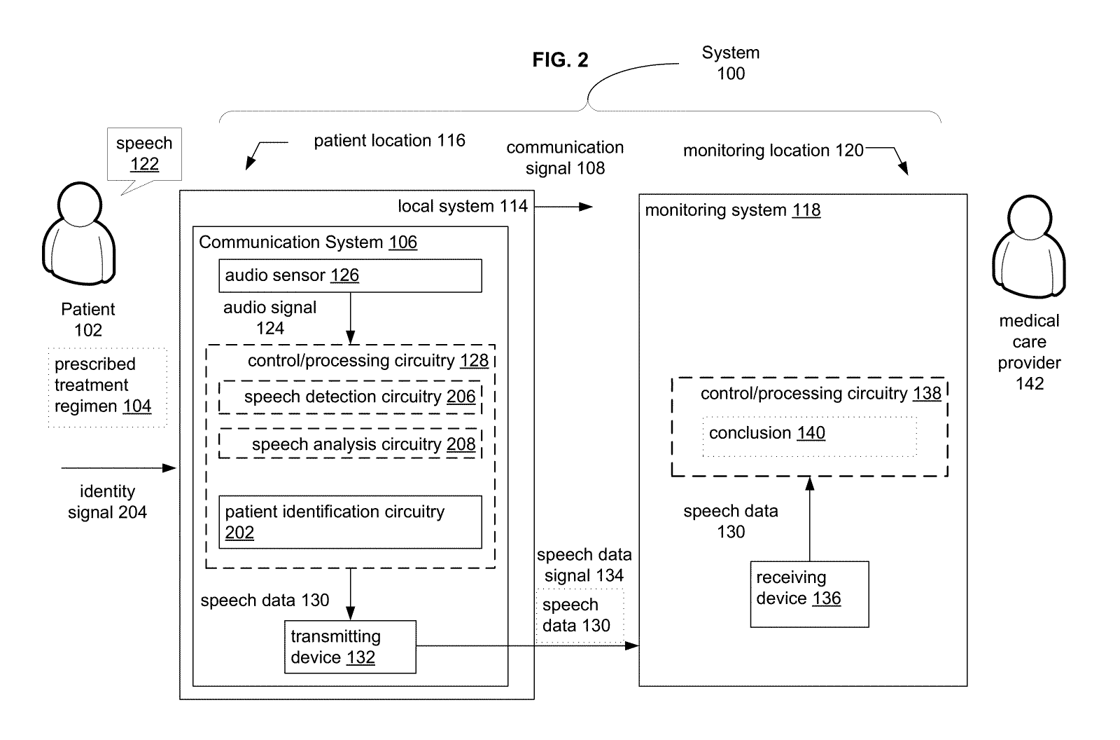

FIG. 2 illustrates basic components of local system 114 at patient location 116. Local system 114 includes at least communication system 106, which includes at least one audio sensor 126 for sensing at least one audio signal 124 during use of communication system 106 by patient 102. Audio signal 124 includes patient speech 122. Local system 114 also includes control/processing circuitry 128 and transmitting device 132. In some aspects, control/processing circuitry 128 is part of communication system 106. In some aspects, some or all of control/processing circuitry 128 is distinct from, but used in combination with, communication system 106, e.g., application software or hardware accessories or add-ons to a cell phone, or a plug in card or accessory device used in combination with a computing system.

Control/processing circuitry 128 includes patient identification circuitry 202, which is configured to determine a presence of patient 102 from at least one identity signal 204 sensed at patient location 116. In some aspects, control/processing circuitry includes speech detection circuitry 206 and speech analysis circuitry 208.

Speech detection circuitry 206 is used for processing the at least one audio signal 124 to identify at least one section of the at least one audio signal containing speech of the patient. In an aspect, speech detection circuitry 206 is configured to identify the at least one section of the at least one audio signal containing speech of the patient based at least in part on the determination of the presence of patient 102 by patient identification circuitry 202. Speech analysis circuitry 208 is used for analyzing the at least one section of the at least one audio signal 124 to generate speech data 130 including data indicative of whether the patient has complied with prescribed treatment regimen 104.

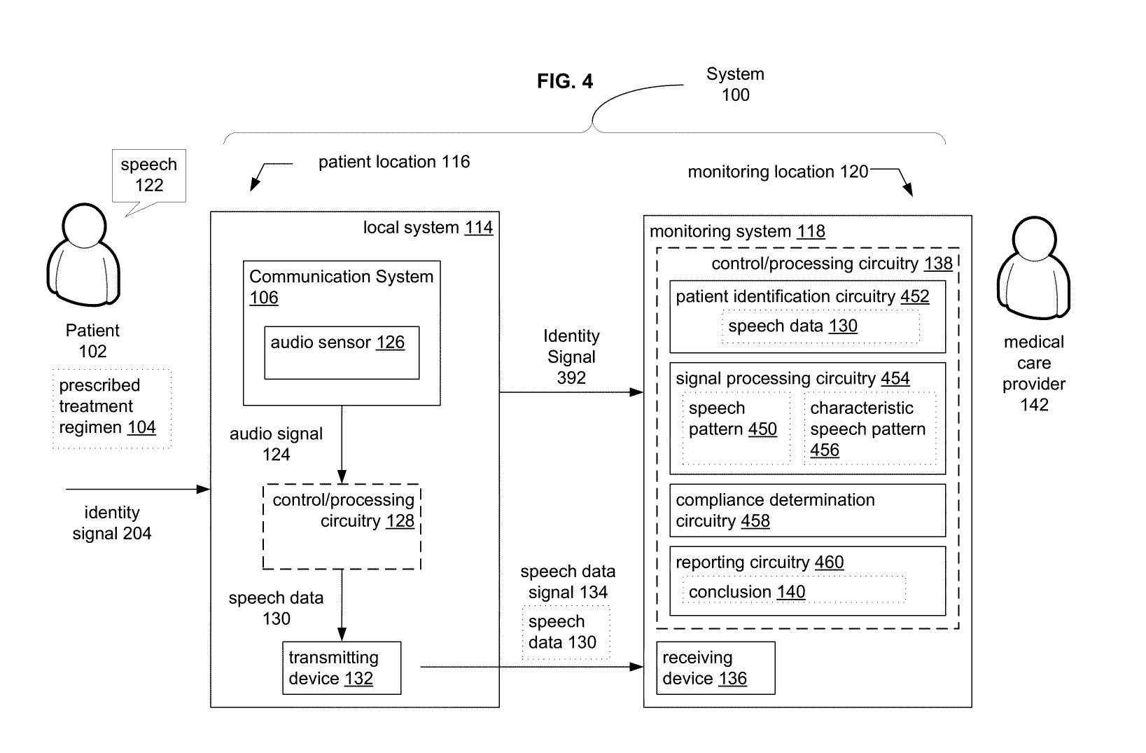

Transmitting device 132 is used for transmitting speech data signal 134 from patient location 116 to receiving device 136 at monitoring location 120. Speech data signal 134 contains speech data 130 including data indicative of whether patient 102 has complied with prescribed treatment regimen 104. Transmitting device 132 can be a component of communication system 106 as depicted in FIG. 2 or a component of local system 108 that is distinct from but used in combination with communication system 106, as depicted in FIG. 1. System components at monitoring location 120 are as described in connection with FIG. 1 or in connection with the example embodiments in FIGS. 4, 5, 8 and 9.

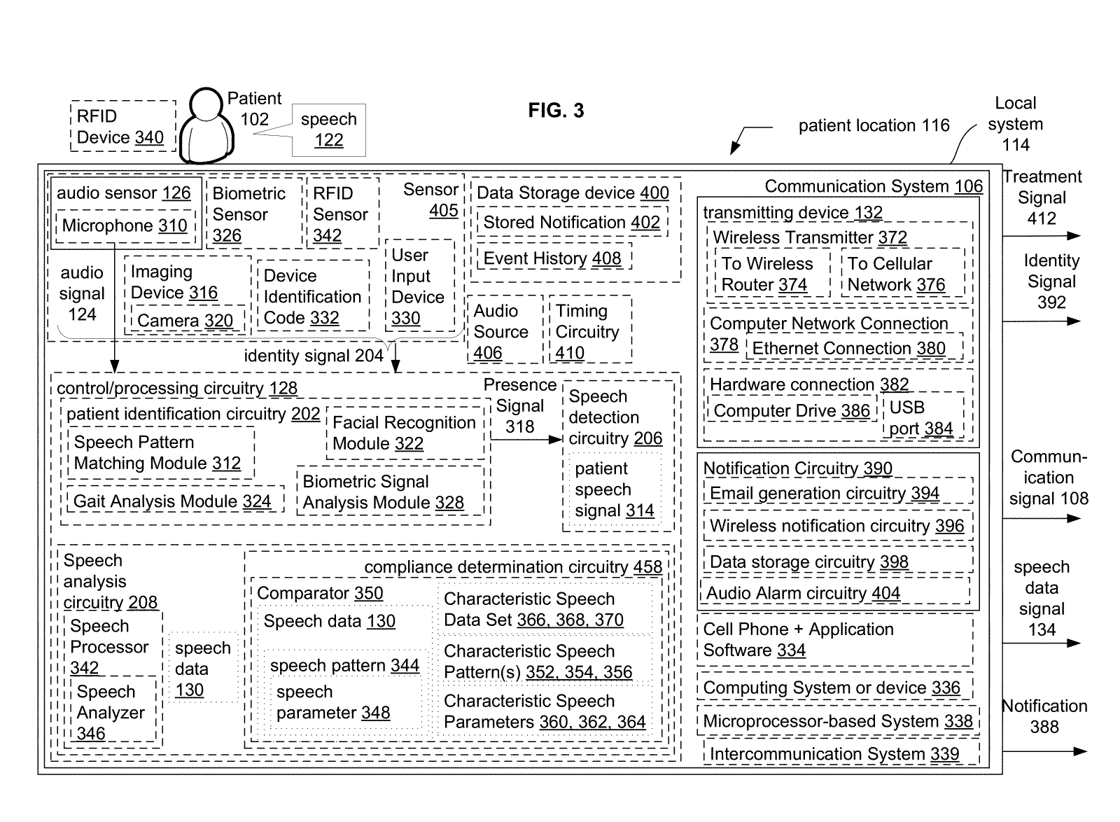

FIG. 3 depicts details of local system 114, showing additional and alternative components. Local system 114 includes communication system 106, which includes one or multiple audio sensors 126, which may be of the same or different types, without limitation. Audio sensor 126 may be, for example, a microphone 310. Audio sensor 126 can be a component of communication system 106, e.g., a microphone built into a cell phone, or a microphone built into or used in combination with a desktop computer or other computing system used for, e.g., voice over internet protocol (VOIP) communications.

Local system 114 also includes at least one transmitting device 132 at patient location 116 for transmitting a speech data signal 134 from patient location 116 to receiving device 136 at monitoring location 120. Speech data signal 134 contains speech data 130 including data indicative of whether patient 102 has complied with prescribed treatment regimen 104. In an aspect, transmitting device 132 is a component of communication system 106, as depicted in FIG. 3. In other aspects, transmitting device 132 is a part of local system 114, distinct from communication system 106 but configured to receive speech data from communication system 106 and subsequently transmit speech data signal 134 to a receiving device (not shown in FIG. 3, but like receiving device 136 at monitoring location 120 as shown in FIG. 2).

In an aspect, communication system 106 also includes patient identification circuitry 202, which is configured to determine a presence of patient 102 from at least one identity signal 204 sensed at patient location 116. The presence and/or identity of the patient can be determined at patient location 116, based on identity signal 204. In some embodiments, identity signal 392, which is transmitted to the monitoring system (not shown in FIG. 3), is the same as identity signal 204. In other aspects, identity signal 392 is a processed version of identity signal 204, or is determined from identity signal 204. In other aspects, identity signal 392 is a signal that is transmitted to the monitoring location and used to determine patient identity at the monitoring location, as an alternative to (or in addition to) determining the patient identity at patient location 116.

In an aspect, at least one identity signal 204 includes at least a portion of the at least one audio signal 124, wherein patient identification circuitry 202 is configured to analyze the at least one audio signal 124 to determine the presence of patient 102 by identifying at least a portion of the at least one audio signal 124 that resembles known speech of the patient, using speech pattern matching module 312, and wherein speech detection circuitry 206 is configured to identify the at least one section of the at least one audio signal 124 containing speech of patient 102 by identifying speech in the at least one audio signal 124 corresponding (e.g. spatially and/or temporally) to presence of patient 102 detected from the at least one audio signal 124. The at least one section of at least one audio signal 124 containing speech of patient 102 is patient speech signal 314. For example, a continuous speech system may be used for identifying the speaker, as described in Chandra, E. and Sunitha, C., “A review on Speech and Speaker Authentication System using Voice Signal feature selection and extraction,” IEEE International Advance Computing Conference, 2009. IACC 2009, Page(s): 1341-1346, 2009 (DOI: 10.1109/IADCC.2009.4809211), which is incorporated herein by reference. In an aspect, patient identification circuitry 202 is configured to analyze audio signal 124 to determine the presence of the patient based on frequency analysis of the audio signal. Magnitude or phase spectral analysis may be used, as described in McCowan, I.; Dean, D.; McLaren, M.; Vogt, R.; and Sridharan, S.; “The Delta-Phase Spectrum With Application to Voice Activity Detection and Speaker Recognition,” IEEE Transactions on Audio, Speech, and Language Processing, 2011, Volume: 19, Issue: 7, Page(s): 2026-2038 (DOI: 10.1109/TASL.2011.2109379), which is incorporated herein by reference. In order to use audio signal 124 as identity signal 204, it may be necessary to process audio signal 124 to determine presence of the patient and simultaneously or subsequently process audio signal 124 with speech detection circuitry 206 to generate patient speech signal 314. This can be accomplished by parallel processing of audio signal 124 by patient identification circuitry 202 and speech detection circuitry 206, or by processing audio signal 124 first with patient identification circuitry 202 and subsequently with speech detection circuitry 206. If the latter approach is used, generation of patient speech signal 314 may not take place strictly in real time. Patient speech signal 314 can be identified through the use of other types of identity signal, as well, as described herein below.

In an aspect, the at least one identity signal 204 includes an image signal received from an imaging device 316 at patient location 116, wherein the patient identification circuitry 202 is configured to analyze the image signal to determine the presence of the patient 102 and generate presence signal 318, and wherein the speech detection circuitry 206 is configured to identify at least one section of the at least one audio signal 124 containing speech of patient 102 by identifying speech in the at least one audio signal 124 corresponding to an image signal representing the patient (patient speech signal 314). In an aspect, imaging device 316 includes a camera 320.

In an aspect, patient identification circuitry 202 is configured to analyze the image signal to determine the presence of the patient through facial recognition, with facial recognition module 322, e.g., using approaches as described in Wheeler, Frederick W.; Weiss, R. L.; and Tu, Peter H., “Face recognition at a distance system for surveillance applications,” Fourth IEEE International Conference on Biometrics: Theory Applications and Systems (BTAS), 2010 Page(s): 1-8 (DOI: 10.1109/BTAS.2010.5634523), and Moi Hoon Yap; Ugail, H.; Zwiggelaar, R.; Rajoub, B.; Doherty, V.; Appleyard, S.; and Hurdy, G., “A Short Review of Methods for Face Detection and Multifractal Analysis,” International Conference on CyberWorlds, 2009. CW '09, Page(s): 231-236 (DOI: 10.1109/CW.2009.47), both of which are incorporated herein by reference. In an aspect, patient identification circuitry 202 is configured to analyze the image signal to determine the presence of the patient through gait analysis, with gait analysis module 324. Identification of the patient based on gait analysis can be performed for example by methods as described in U.S. Pat. No. 7,330,566, issued Feb. 12, 2008 to Cutler, and Gaba, I. and Kaur P., “Biometric Identification on The Basis of BPNN Classifier with Other Novel Techniques Used For Gait Analysis,” Intl. J. of Recent Technology and Engineering (IJRTE) ISSN: 2277-3878, Vol. 2, issue 4, September 2013, pp. 137-142, both of which are incorporated herein by reference.

In an aspect, the at least one identity signal includes a biometric signal from at least one biometric sensor 326 at patient location 116, wherein patient identification circuitry 202 is configured to analyze the biometric signal, using biometric signal analysis module 328 to determine the presence of the patient, and wherein the speech detection circuitry 206 is configured to identify at least one section of the at least one audio signal containing speech of the patient by identifying speech in the at least one audio signal 124 corresponding to a biometric signal representing patient 102. Biometric identification can include face and gait recognition, as described elsewhere herein, and recognition based on a variety of other physiological or behavioral characteristics, such as fingerprints, voice, iris, retina, hand geometry, handwriting, keystroke pattern, etc., e.g., as described in Kataria, A. N.; Adhyaru, D. M.; Sharma, A. K.; and Zaveri, T. H., “A survey of automated biometric authentication techniques” Nirma University International Conference on Engineering (NUiCONE), 2013, Page(s): 1-6 (DOI: 10.1109/NUiCONE.2013.6780190), which is incorporated herein by reference. U.S. Pat. No. 8,229,178 issued Jul. 24, 2012 to Zhang et al., which is incorporated herein by reference, describes a method for acquiring a palm vein image with visible and infrared light and extracting features from the image for authentication of individual identity. Biometric identification can be based on imaging of the retina or iris, as described in U.S. Pat. No. 5,572,596 issued to Wildes et al. on Nov. 5, 1996 and U.S. Pat. No. 4,641,349 issued to Flom et al. on Feb. 3, 1987, each of which is incorporated herein by reference. Combinations of several types of identity signals can also be used (e.g., speech and video, as described in Aleksic, P. S. and Katsaggelos, A. K. “Audio-Visual Biometrics,” Proceedings of the IEEE Volume: 94, Issue: 11, Page(s): 2025-2044, 2006 (DOI: 10.1109/JPROC.2006.886017), which is incorporated herein by reference).

In an aspect, identity signal 204 includes at least one authentication factor, including, for example, a security token, a password, a digital signature, or cryptographic key. In an aspect, an authentication factor is received by communication system 106 via a user input device 330. User input device 330 can include various types of user input devices or controls as are well known to those of ordinary skill in the art, including but not limited to keyboards, touchpads, touchscreen, mouse, joystick, microphone or other voice input, buttons, or switches. User input device 330 can be integral to a communication device, e.g. a key pad of a cell phone. One or more user input device 330 in local system 114 can be used to receive various types of user inputs relating to operation of local system 114, not limited to entry of an authentication factor.

In another aspect, identity signal 204 includes a device identification code 332, which identifies a device or component of local system 114. Device identification code 332 may be, for example, a cell phone identification code, such as an electronic serial number, a mobile identification number, or a system identification code. In various aspects, device identification code 332 identifies a cell phone (including application software) 334, a computing system or device 336, or a stand-alone microprocessor-based system 338, or a component thereof. Device identification code 332 can serve to identify patient 102 providing the identified device, for example a personal computer or cell phone, is consistently used only by patient 102. Identifying patient 102 based on device identification code 332 may be done, for example, if some or all components of local system 114 are shared by multiple users but the device or component associated with device identification code 332 is used consistently by patient 102. As with other types of identity signals, device identification code 332 (or a value derived therefrom) can be transmitted to the monitoring system as identity signal 392.

In an aspect, identity signal 204 includes a radio frequency identification (RFID) signal, e.g., from an RFID device 340, which may be carried, worn by, or otherwise associated with patient 102 and sensed by RFID sensor 342. In an aspect, RFID device 340 is a passive RFID in a tag or chip associated with the patient. In an aspect, RFID sensor 342 is an active RFID reader.

In general, identity signal 204 is sensed with one or more sensor 405. Audio sensor 126, imaging device 316, biometric sensor 326, user input device 330, and RFID sensor 342 are examples of sensors that may be used to sense identity signal 304. Other types of sensor 405 may be used, as well.

In an aspect, patient identification circuitry 202 is configured to distinguish the presence of patient 102 from the presence of another individual. For example, in an aspect, patient 102 is the normal user of communication system 106, and speech patterns of patient 102 during using of the communication system 106 are used to assess compliance of patient 102 with a prescribed treatment regimen. In the event that another individual uses the cell phone belonging to patient 102, speech patterns detected from the other individual should not be used to determine the compliance of patient 102. In an aspect, patient identification circuitry 202 is configured to determine the presence of the patient by determining that information contained in the identity signal matches patient information associated with the patient. For some types of identity signal (e.g., a password or device identity code), an exact match can be obtained. In other cases, a match is obtained by using a windowing, thresholding, or distance measurement to determine whether the identity signal (or information contained there) matches sufficiently closely patient information associated with the patient. In an aspect, patient identification circuitry 202 is configured to distinguish the presence of the patient from the absence of the patient (e.g., to determine whether patient 102 is present and speaking, versus background noise detected by audio sensor 126 that does not contain speech of patient 102).

In an aspect, patient identification circuitry 202 generates presence signal 318 to indicated presence and/or identity of patient 102. In an aspect, presence signal 318 is provided as an input to speech detection circuitry 206. Presence of patient 102 may be indicated by a value of presence signal 318. For example, in some aspects, presence signal 318 is a binary signal; e.g., presence signal 318 has a high value if the patient is present or a low value if the patient is not present (or vice versa). In an aspect, patient speech signal 314 is acquired from audio signal 124 only when the value of presence signal 318 indicates that patient 102 is present. Alternatively, in some aspects presence signal 318 is a continuous valued signal that indicates the probability that the patient is present. For example, presence signal 318 has a value of 100 if there is 100 percent probability that the patient is present, a value of zero if there is zero percent probability that the patient is present, or an intermediate value if there is an intermediate probability that the patient is present. It will be appreciated that in some contexts, the determination of whether the patient is present or absent will be relatively straightforward, in which case a binary presence signal may be appropriate, whereas in others (e.g., in cases where the presence of the patient must be distinguished from the presence of other individuals, e.g., from a conference call) there is some likelihood of error in identifying the presence of the patient (with the likelihood of error potentially dependent upon the number and identity of the other individuals present), such that an indication of the probability that the patient is present may be more appropriate. In some aspects, various device functions (e.g., acquisition of speech data, performance of speech analysis, or transmission of speech data signal 134 to the monitoring location) are initiated in response to detection of the presence of patient 102. In some aspects, presence of patient 102 is a necessary but not sufficient condition for performance of particular device functions. For example, data may be collected at certain times of day, contingent upon the presence of patient 102. In another aspect, data is collected when patient 102 is present and initiates a VOIP communication session or phone call, for example.

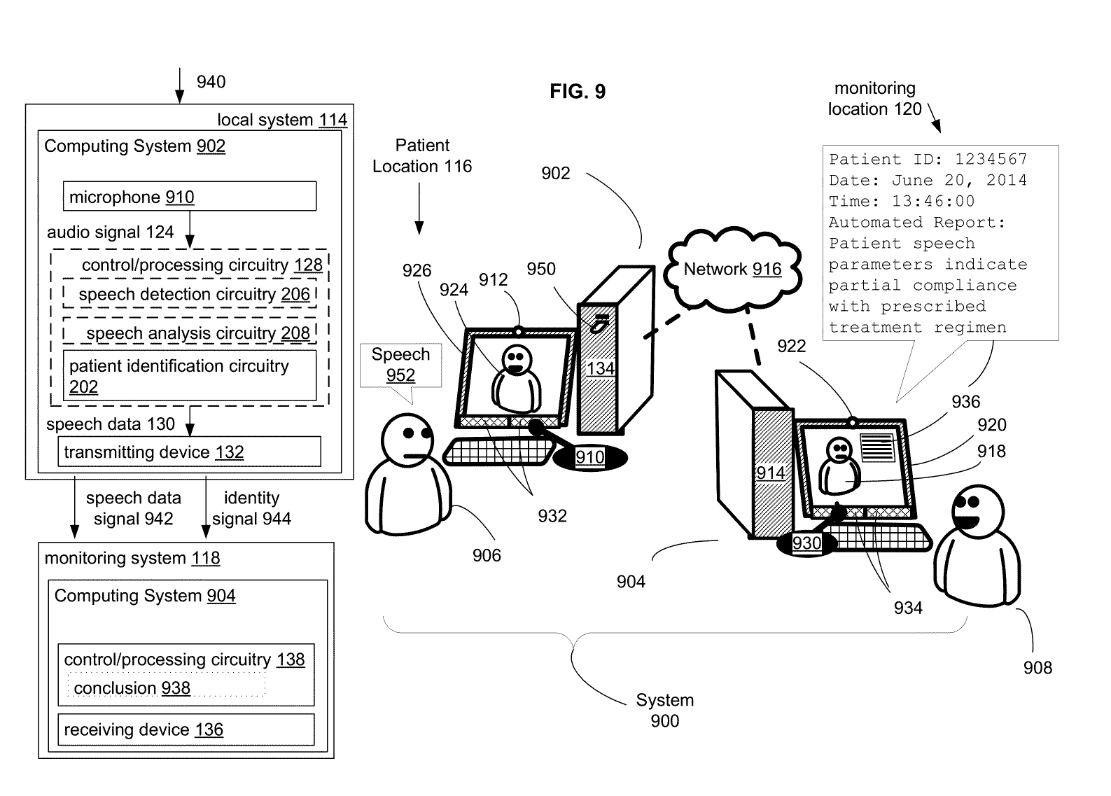

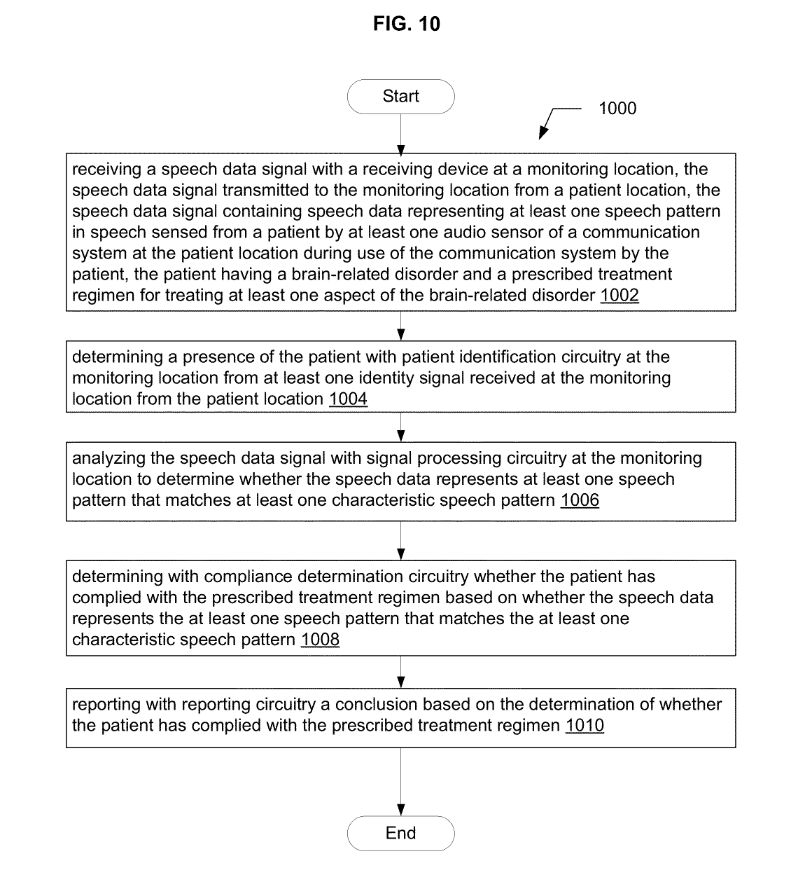

Local system 114 can be constructed and implemented in a variety of embodiments in which different devices and/or device components provide the functionality described herein. For example, in various aspects, audio sensor 126, control/processing circuitry 128, and transmitting device 132 may be components of a cell phone configured with application software, as indicated at 334, or a computing system or device, as indicated at 336, examples of which are shown in FIGS. 9 and 10. In other aspects, local system 114 can include a microprocessor-based system 338, which may be, for example, a stand-alone device constructed specifically as a combination communication and compliance monitoring device. In an aspect, audio sensor 126, control/processing circuitry 128, and transmitting device 132 are components of an intercommunication (“intercom”) system 339.

In an aspect, local system 114 includes data storage device 400, which may be any of various types of data storage and/or memory devices. Local system 114 may include one or more power source (not shown), e.g., a battery, a plug for connecting to an electrical outlet or communication port, e.g., a USB port, or any of various other types of power sources.

Local system 114 includes speech detection circuitry 206 in communication with system 106. Speech detection circuitry 206 is used for processing the at least one audio signal 124 to identify at least one section of the at least one audio signal 124 containing speech of the patient (e.g., patient speech signal 314), wherein the speech detection circuitry 206 is configured to identify the at least one section of the at least one audio signal containing speech of the patient based at least in part on the determination of the presence of patient 102 by patient identification circuitry 202. In an aspect, speech detection circuitry 206 processes the at least one audio signal to exclude at least one portion of the at least one audio signal that does not contain speech of the patient.

Communication system 106 includes speech analysis circuitry 208 for analyzing the at least one section of the at least one audio signal 124 (e.g., patient speech signal 314) to generate speech data 130, which includes data indicative of whether the patient has complied with prescribed treatment regimen 104. As used herein, “speech data” may refer to any or all of a digitized audio signal containing one or more speech-containing portions and one or more non-speech-containing portions, a digitized audio signal from which non-speech-containing portions have been removed to leave one or more speech-containing portions, speech pattern data derived or computed from a digitized audio signal containing speech, or speech parameter data derived or computed from a digitized audio signal containing speech, for example. “Speech data” may include several types of data, e.g., one or more digitized audio speech signal, one or more speech pattern, and/or one or more speech parameter. Speech data may be said to represent a speech pattern if it includes the speech pattern and/or one or more digitized audio signal, speech parameter, or other data from which the speech pattern can be derived or computed. Speech data representing a speech pattern may include data in addition to the speech pattern and/or data from which the speech pattern can be derived or computed.

In an aspect, speech analysis circuitry 208 includes a speech processor 342 for processing at least one section of the at least one audio signal 124 to determine at least one speech pattern 344 of the patient. In an aspect, speech data 130 includes the at least one speech pattern 344 of the patient.

A speech pattern can be defined as a consistent, characteristic form, style, or method of speech comprising a distribution or arrangement of repeated or corresponding parts composed of qualities, acts, or tendencies. In an embodiment a speech pattern can include one or more qualities of diction, elocution, inflection, and/or intonation. In an embodiment a speech pattern can include aspects of language at the lexical level, sentential level, or discourse level. In an embodiment, a speech pattern may conform to the Thought, Language, and Communication Scale and/or Thought and Language Index. Reviews describing speech patterns and linguistic levels and the tools used to study them include Covington M. A., et al. “Schizophrenia and the structure of language: The linguist's view,” Schizophrenia Research 77: 85-98, 2005, and Kuperberg and Caplan (2003 Book Chapter: Language Dysfunction in Schizophrenia), which are both incorporated herein by reference.

In an embodiment a speech pattern includes a linguistic pattern determined at the lexical level. A speech pattern may include a frequency of, for example, pauses, words, or phrases. For example a speech pattern may include a frequency of pauses. A higher frequency of pauses or reduced verbal fluency can be indicative of alogia associated with a brain disorder, e.g., bipolar disorder, depression, or schizophrenia. For example, a speech pattern may include a frequency of dysfluencies (“uhs” and “ums”). A higher than average frequency of dysfluencies may indicate a slowed speech, the inability to think clearly, or a deliberate attempt to appear unaffected by illness, all of which have been associated with psychological pathologies. For example, a speech pattern may include a distribution of pauses and dysfluencies. A high frequency and particular distribution of pauses and dysfluencies may be indicative of anomia associated with schizophrenia or with an aphasia due to brain injury. For example, a speech pattern may include a frequency of neologisms and/or word approximations, or glossomania. Higher than average frequencies of neologisms and/or word approximations, or glossomania, have been associated with disorders such as schizophrenia, schizoaffective disorder, or mania. For example a speech pattern may include a frequency of word production. A frequency of word production lower than the norm may be indicative of a brain disorder such as schizophrenia. An excessive speed during speech, as in pressured speech, may be indicative of a brain disorder such as the mania of bipolar disorder, while reduced speed may be indicative of depression or a depressive episode. For example, a pattern may include a type:token ratio (i.e., number of different words (types) in relation to the total number of words spoken (tokens)). A type:token ratio that is generally lower than the norm can be indicative of schizophrenia. For example, a speech pattern may include a frequency of specific words. Quantitative word counts have been used as a tool in the identification and examination of abnormal psychological processes including major depression, paranoia, and somatization disorder. A high frequency of negative emotion words or death-related words may be indicative of depression. Psychologically relevant words can include those listed in one or more dictionaries of the Linguistic Inquiry and Word Count (LIWC) program (see Tausczik and Pennebaker, “The Psychological Meaning of Words: LIWC and Computerized Text Analysis Methods,” Journal of Language and Social Psychology 29(1): 24-54, 2010, which is incorporated herein by reference). Words interpreted as carrying normative emotional qualities are found in dictionaries of two programs, Affective Norms for English Words (ANEW) and Dictionary of Affect in Language (DAL) (see Whissell C., “A comparison of two lists providing emotional norms for English words (ANEW and the DAL),” Psychol Rep., 102(2):597-600, 2008, which is incorporated herein by reference).

In an embodiment a speech pattern includes a linguistic pattern determined at the sentential level or discourse level. For example, a speech pattern can include a consistent grammatical style. A pattern comprising a style that is grammatically deviant from the norm might include the overuse of the past tense, indicating detachment from the subject being discussed. A pattern comprising a style that is grammatically deviant from the norm, e.g., as reflected by a higher percentage of simple sentences and, in compound sentences, fewer dependent clauses may be indicative of schizophrenia. For example, a speech pattern may include a ratio of syntactic complexity (number of clauses and proportion of relative:total clauses). An abnormal ratio may indicate a brain disorder. For example, a speech pattern may include a frequency of subordinate clauses. An increase in subordinate clauses has been observed in the speech of psychopaths (see, e.g., Hancock et al., “Hungry like the wolf: A word-pattern analysis of the language of psychopaths,” Legal and Criminological Psychology, 2011; DOI: 10.1111/j.2044-8333.2011.02025.x, which is incorporated herein by reference). For example, a speech pattern may include a relatedness of lexical content such as semantic or sentential priming. A speech pattern of abnormal priming may indicate a brain disorder such as schizophrenia. For example, a speech pattern may include a frequency of one or more use of cohesive ties, e.g., as demonstrated by references, conjunctions, or lexical cohesion. A low frequency of reference ties has been observed in patients suffering from schizophrenia. For example, a speech pattern may include an hierarchical structure within a discourse, e.g., a systematic structure in which propositions branch out from a central proposition. A speech pattern lacking a systematic structure may be indicative of schizophrenia.

For example, a speech pattern including a linguistic pattern determined at the sentential level or discourse level may include a representation of content of thought (what the patient is talking about). For example, a speech pattern may include a representation of form of thought (the way ideas, sentences, and words are put together). A speech pattern containing representations of content or form of thought that differ from those expected (e.g., as determined from population patterns) may indicate a psychological disorder such as schizophrenia. Examples of representations of content or form of thought observed in schizophrenia include derailment, loss of goal, perseveration, and tangentiality. For example, a speech pattern may include aspects of linguistic pragmatics (e.g., cohesion or coherence). Abnormal patterns in pragmatics may be indicative of a brain disorder such as schizophrenia or mania. Examples of speech patterns and content of thought are discussed by Covington, et al., idem, and by Kuperberg and Caplan idem. A program for classifying parts of speech (e.g., noun, verb, adjective, etc.) based on the surrounding context and analysis of semantic content has been developed and is available under the Wmatrix interface (http://ucrel.lancs.ac.uk/wmatrix/) and has been used to analyze the speech of psychopaths (see Hancock, idem).

In an embodiment, a speech pattern includes an acoustic quality. In an embodiment, a speech pattern includes volume. For example, excessive or reduced volume may be indicative of a symptom of a brain disorder. In an embodiment, a speech pattern includes prosody (the rhythm, stress, and intonation of speech). For example, aprosody or flattened intonation can be indicative of schizophrenia. In an embodiment, a speech pattern includes a voice quality of phonation. In an embodiment, a speech pattern includes pitch or timbre. For example, abnormalities in pitch have been observed in schizophrenics. For example, a strained quality, choking voice, or creaking voice (laryngealisation) may be indicative of a psychological disorder. Voice qualities and volume in linguistics are discussed by Covington, idem.

In an aspect, the at least one speech pattern 344 is represented in speech data 130 in numerical or categorical form. For example, a speech pattern represented in numerical form may include one or more numerical values representing one or more speech parameters. Particular speech parameters represented in a speech pattern may be selected for the purpose of evaluating/monitoring particular brain-related disorders. For example, in an aspect a speech pattern for evaluating/monitoring depression includes values representing the following parameters: speech volume, frequency of word production, frequency of pauses, and frequency of negative value words. In another aspect, a speech pattern for evaluating/monitoring schizophrenia includes values representing frequency of word production, frequency of pauses, frequency of disfluencies, type:token ratio, and speech volume. A speech parameter or pattern may be represented in speech data 130 in categorical form; for example, frequency of word production may be categorized as low, medium, or high rather than represented by a specific numerical value.

In an aspect, speech analysis circuitry 208 includes compliance determination circuitry 458, including one or more comparator 350. Comparator 350 is used for comparing the at least one speech pattern 344 with at least one characteristic speech pattern 352 to determine whether the patient has complied with the prescribed treatment regimen. In some aspects, comparator 350 is configured to compare the at least one speech pattern 344 with a plurality of characteristic speech patterns, e.g., characteristic speech patterns 352, 354, and 356 (three are shown in the example of FIG. 3, but a comparison can be made with any number of characteristic speech patterns). In an aspect, speech analysis circuitry 208 is configured to generate speech data 130 including data indicative of whether the patient has complied with the prescribed treatment regimen based upon whether the at least one speech pattern 344 matches at least one of the plurality of characteristic speech patterns (e.g., characteristic speech patterns 352, 354, and 356).

In various aspects, speech analysis circuitry 208 is configured to determine that the patient has failed to comply with the prescribed treatment regimen, or to determine that the patient has complied with the prescribed treatment regimen. Determination of compliance may be accomplished by a thresholding, windowing, or distance computation of one or multiple parameters relative to characteristic threshold or range values for the parameter. For example, for a given parameter, a patient parameter value higher than a characteristic threshold value may indicate compliance of the patient with the prescribed treatment regimen, while a patient parameter value equal to or lower than the threshold value may indicate non-compliance. As another example, a patient parameter value that lies within a range of characteristic values for the parameter may indicate compliance, while a patient parameter value outside the range of characteristic values indicates non-compliance. Speech analysis circuitry 208 may utilize various types of distance computations to determine whether patient parameter values are within a threshold distance or distance range from characteristic values. Distance computations based on one or more parameters or data values are known (including, but not limited to, least-squares calculations). In an aspect, speech analysis circuitry 208 is configured to determine whether the patient has complied with the prescribed treatment regimen based upon a determination of whether the speech corresponds to at least one of a plurality of characteristic speech patterns. For example, the plurality of characteristic speech patterns can include multiple characteristic speech patterns, each corresponding to a patient speech pattern obtained at a different treatment regimen, for example, a different dose of a drug. By identifying which characteristic speech pattern the patient speech pattern matches or is closest to, the drug dose taken by the patient can be determined. For example, the patient may have taken the drug, but at a lesser dose or less often than was prescribed. Accordingly, the patient's speech pattern will then match the characteristic speech pattern associated with the lesser dose of drug, indicating partial, but not full, compliance of the patient with the prescribed treatment regimen.

In an aspect, speech processor 342 includes speech analyzer 346 for assessing the at least one speech pattern 344 to determine at least one speech parameter 348 indicative of whether the patient has complied with the prescribed treatment regimen, wherein speech data 130 includes the at least one speech parameter 348. Speech parameters include, but are not limited to, measures of prosody, rhythm, stress, intonation, variance, intensity/volume, pitch, length of phonemic syllabic segments, and length of rising segments, for example. In an aspect, speech data 130 includes at least one speech parameter 348, which may include, for example, one or more of prosody, rhythm, stress, intonation, variance, intensity/volume, pitch, length of phonemic syllabic segments, and length of rising segments. In an aspect, comparator 350 is configured for comparing at least one speech parameter 348 of the patient with at least one characteristic speech parameter 360 to determine whether the patient has complied with the prescribed treatment regimen. In an aspect, comparator 350 is configured to compare at least one speech parameter 348 of the patient with a plurality of characteristic speech parameters (for example, characteristic speech parameters 360, 362, and 364) to determine whether the patient has complied with the prescribed treatment regimen. For example, in an aspect, the result of such a comparison is either “patient has complied” or “patient has not complied.” In an aspect, comparator 350 is configured to compare at least one speech parameter 348 of the patient with plurality of characteristic speech parameters 360, 362, and 364 to determine a level of compliance of the patient with the prescribed treatment regimen. Determination of compliance, non-compliance, or level of compliance may be performed with the use of comparator 350 using thresholding, windowing, or distance measurements, for example, as described herein above. In an aspect, speech analysis circuitry 208 is configured to generate speech data 130 including data indicative of whether the patient has complied with the prescribed treatment regimen based upon whether the at least one speech parameter 348 matches at least one of the plurality of characteristic speech parameters (e.g., characteristic speech parameters 360, 362, and 364). Similarly, determination of compliance or non-compliance of patient 102 with a prescribed treatment regimen maybe be accomplished with the use of comparator 350 for various types of speech data by comparing speech data 130 with one or more characteristic speech data sets 366, 387, 370, using approaches as described herein above. In an aspect, speech analysis circuitry 208 is configured to generate speech data 130 including data indicative of whether the patient has complied with the prescribed treatment regimen based upon whether the speech data matches at least one of the plurality of characteristic speech data sets (e.g., characteristic speech data sets 366, 387, and 370).

Local system 114 includes transmitting device 132, which in various aspects includes a wireless transmitter 372, which may be configured to transmit signals to a wireless router 374 or cellular network 376, for example. Transmitting device 132 receives speech data 130 from control/processing circuitry 128 and generates speech data signal 134 suitable for transmission to receiving device 136. In another aspect, transmitting device 132 includes a computer network connection 378, e.g., an Ethernet connection 380, or a hardware connection 382, for example a communication port 384 (which may be, for example, a USB port, Type-A or Type-B port, Micro-USB, or Mini-USB Port or other type of communication port) or computer drive 386. Transmitting device 132 functions to transmit speech data signal 134, which includes speech data 130, but may also be used to transmit communication signal 108, notification 388 generated by notification circuitry 390, identity signal 392 (which may be the same as identity signal 204, or a processed version thereof), and other data, instructions, or information, for example, as discussed elsewhere herein. In some aspects, transmitting device 132 provides for two-way communication between local system 114 and the monitoring system (e.g., monitoring system 118 as shown in FIG. 1), and one-way or two-way communication between local system 114 and other systems or devices located remotely from local system 114, including but not limited to a cell phone or other communication device at destination 110, as shown in FIG. 1. Communication system 106 may include more than one transmitting device, and may include more than one type of transmitting device.

In an aspect, local system 114 also includes notification circuitry 390 for generating a notification. Notification circuitry 390 may include, for example, email generation circuitry 394 for generating an email notification, wireless notification circuitry 396 for generating a notification to be transmitted to a wireless device, or data storage circuitry 398 for storing a notification in a data storage device (e.g., data storage device 400). Notifications may be transmitted via transmitting device 132 or other transmitting devices.

Various aspects of system functionality can be distributed between local system 114 and monitoring system 118. With regard to processing of speech signals, if the majority of speech processing is to take place in monitoring system 118, speech data 130 transmitted in speech data signal 134 may include minimally processed patient speech data. On the other hand, if the majority of speech processing is performed in local system 114, speech data signal 134 may contain speech data 130 that includes processed patient speech data (e.g., speech patterns and/or parameters). However, even if speech processing is performed in local system 114, speech data 130 may include both processed and unprocessed patient speech data (e.g., raw speech data as well as speech parameters and or speech patterns) to be transmitted in speech data signal 134.

In some aspects, patient speech data (e.g., patient speech signal 314) is compared directly with characteristic speech data sets, rather than being processed first by speech processor 342 to determine speech pattern 344. For example, comparator 350 in speech analysis circuitry 208 compares patient speech data 130 with one or multiple characteristic speech data sets 366, 368, and 370 indicative of the characteristic speech pattern, where each said characteristic speech data set is indicative of a characteristic speech pattern.

In the above scenarios, the result of the comparison performed by comparator 350 is a determination that the patient speech data (or patient speech pattern or speech parameter derived therefrom) either does, or does not, match one or more characteristic speech data sets, patterns, or parameters. In an aspect, if there is a match, notification 388 is generated by notification circuitry 390 regarding whether the patient has complied with the prescribed treatment regimen. In practice, the comparison performed by comparator 350 (which may include thresholding, windowing, distance computation, for example, as discussed herein above) will result in production of a signal that indicates at least whether the patient has complied with the prescribed treatment regimen, and alternatively, or in addition, a level of compliance with the prescribed treatment regimen. In some cases, a medical care provider at the monitoring location (or another party concerned with the patient's health and well-being, such as a parent, family member, caretaker, healthcare provider) is notified only if the patient has failed to comply with the prescribed treatment regimen. Alternatively, in some aspects the medical care provider is notified when the patient is in compliance with the prescribed treatment regimen. In some aspects, notification can be provided by transmitting a notification 388 generated by notification circuitry 390 and transmitted to the monitoring location by transmitting device 132 or to a wireless device, e.g., a remote device at the patient location, using wireless notification circuitry 396. It will be appreciated that in various aspects, speech analysis circuitry 208 can be configured to determine both compliance and non-compliance, and additionally, or alternatively, level of compliance (either at specific levels or simply partial compliance).

Compliance or lack thereof can be represented by appropriate text or numerical value in a displayed report or email, e.g., reported by notification circuitry 390, or represented by a binary value in data stored by data storage device 400. Alternatively, or in addition, level of compliance can be represented by a continuous value (e.g., percent compliance) or a text descriptor selected from a number of text descriptors corresponding to different levels of compliance (e.g., non-compliance, low compliance, intermediate compliance, near-full compliance, full compliance). Notification circuitry 390 provides for formatting data included in notification 388 appropriately (e.g., by including appropriate text to accompany numerical data values) and for deciding whether and how to report the conclusion, based upon user preferences. For example, who is notified (patient versus medical care provider versus family member) or how notification is provided (stored in an event record, via email, or via a text message to a cell phone) may depend on the patient's level of compliance and the specifics of the patient. In some aspects, notification circuitry 390 can generate different levels of notifications depending on how serious a problem non-compliance is likely to be for the patient. Generating a notification may include retrieving a stored notification 402 from data storage device 400, e.g., selected from among one or more notifications 402 stored in data storage device 400. Notifications may take the form of text or numerical codes, for example.

In an aspect, notification circuitry 390 includes audio alarm circuitry 404 for generating an audio alarm, e.g., a tone or voice alert be delivered via audio source 406 (e.g., a speaker, bell, buzzer, beeper, or the like). In an aspect, notification circuitry 390 provides a notification to patient 102, e.g., by generating an audio alarm via audio source 406 or displaying a text message on a display of, e.g., cell phone 334 or computing system 336. A notification to the patient could take the form of a reminder to take a medication or contact a medical care provider, for example. In another aspect, notification circuitry 390 uses wireless notification circuitry 396 to transmit notification (e.g., via wireless transmitter 372) to a wireless device such as a pager, cell phone, or other wireless device used by a medical care provider or family member interested in tracking the status of the patient. In another aspect, notification circuitry 390 includes data storage circuitry 398 for storing a notification in a data storage device 400. For example, data storage device 400 may provide for storage of a notification in event history 408 in conjunction with information regarding the time at which the notification was generated, obtained, for example from timing circuitry 410. Information stored in event history 408 may be a part of the subject's electronic medical records, and may ultimately be transferred to the monitoring system or other location. In an aspect, timing circuitry 410 includes a clock and/or timer, for example.

FIG. 4 provides an overview of an embodiment of a monitoring system 118 at monitoring location 120. Monitoring system 118 includes at least one receiving device 136 for receiving speech data signal 134 transmitted to monitoring location 120 from patient location 116. As discussed previously, speech data signal 134 contains speech data 130. In an aspect, speech data 130 represents at least one speech pattern 450 in speech sensed from a patient 102 with at least one audio sensor 126 in communication system 106. Speech is sensed from patient 102 during use of communication system 106 by patient 102.

In an aspect, monitoring system 118 includes patient identification circuitry 452 configured to determine a presence of the patient from at least one identity signal 392 received at the monitoring location 120 from the patient location 116. Identity signal 392 may be received by receiving device 136 or, in some aspects, identity signal 392 is received by another receiving device. In some aspects, identity signal 392 is the same as identity signal 204. In an aspect, patient presence is not determined by local system 114, but identity signal 204 received at patient location 116 is transmitted to monitoring system 118, as identity signal 392, along with speech data signal 134, and presence of patient 102 is determined at monitoring location 120. Alternatively, in some aspects, identity of the patient is determined at the patient location 116 by local system 114, based on identity signal 204, as described herein above in connection with FIG. 3, but identity signal 204 is also provided to monitoring system 118, as identity signal 392. In some aspects, identity signal 392 is different from identity signal 204. In some cases identity signal 392 is a processed version of identity signal 204 or is derived from identity signal 204. Processing of identity signal 204 at monitoring system 118 is discussed in greater detail herein below.

In an aspect, monitoring system 118 includes signal processing circuitry 454 configured to analyze speech data signal 134 to determine whether the speech data 130 is representative of the at least one speech pattern 450 that matches at least one characteristic speech pattern 456, and compliance determination circuitry 458 configured to determine whether patient 102 has complied with the prescribed treatment regimen 104 based upon whether speech data 130 represents the at least one speech pattern 450 that matches the at least one characteristic speech pattern 456. In an additional aspect, monitoring system 118 includes reporting circuitry 460 configured to report a conclusion 140 based on the determination of whether the patient has complied with the prescribed treatment regimen.

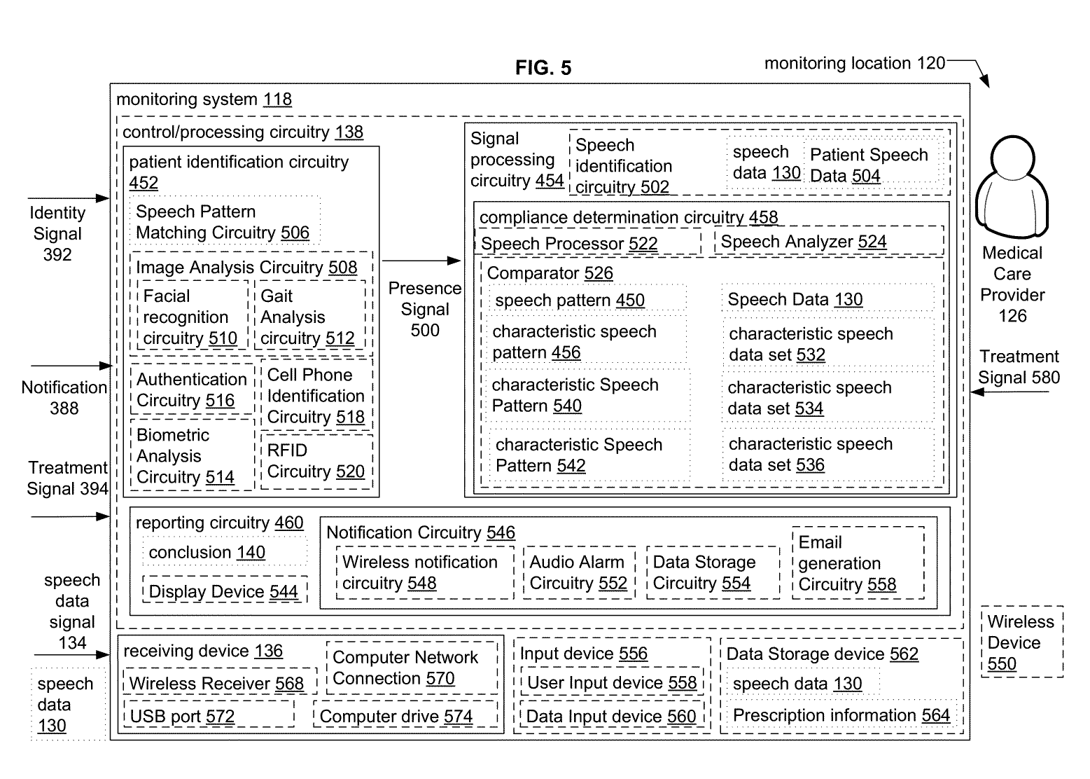

FIG. 5 provides greater detail regarding various aspects of monitoring system 118, including additional and alternative aspects. Monitoring system 118 includes patient identification circuitry 452, which is configured to determine a presence of the patient at the patient location (e.g., determining that the patient is present at local system 114 as shown in FIG. 4) from at least one identity signal 392 received at the monitoring location 120 from the patient location 116 in FIG. 4. As noted herein above, identity signal 392 may be the same as the identity signal detected at the patient location, or may be derived from the identity signal detected at the patient location (e.g., a processed version thereof). In general, identity signal 392 can be any of various types of identity signal, for example as described in connection with FIG. 3. As noted in connection with FIG. 4, identity signal 392 may be received by receiving device 136, or by a separate receiving device. The output of patient identification circuitry 452, determined from the identity signal 392, is presence signal 500, which indicates the presence of the patient at the patient location. Alternatively, or in addition, in an aspect, identity signal 392 is determined with control/processing circuitry at the patient location (i.e., presence signal 318 in FIG. 3) and indicates the presence of a patient of a particular identity at the patient location. In this case, identity signal 392 is sufficient to determine presence signal 500.

In an aspect, monitoring system 118 includes speech identification circuitry 502, which is configured to identify patient speech data 504 in speech data 130 based at least in part on the determination of the presence of the patient by the patient identification circuitry 452. As described in connection with FIG. 3, in some aspects the sensed audio signal is processed such that speech data 130 in speech data signal 134 transmitted from the patient location is limited to patient speech data (e.g., corresponding to patient speech signal 314 in FIG. 3). In other aspects, speech data signal 134 includes speech data 130 that is not limited to patient speech data 504. For example, this may be the case if data signal 134 includes speech data from individuals other than the patient, or if data signal 134 includes patient speech data 504 in combination with non-speech data. In particular, in some aspects, the local system at the patient location (e.g., local system 114 as shown in FIG. 1) does not perform extensive processing prior to transmission of speech data signal 134 (or, alternatively, transmits both processed and unprocessed data in speech data signal 134) and monitoring system 118 performs processing of speech data signal 134 to identify patient speech data 504. In such cases, as shown in FIG. 5, patient identification circuitry 452 is used to identify patient speech data 504.

In an aspect, identity signal 392 includes at least a portion of the speech data signal 134, and patient identification circuitry 452 is configured to analyze the speech data signal 134 to determine, with the use of speech pattern matching circuitry 506, at least a portion of the speech data signal 134 that contains speech data representing a speech pattern that matches a known speech pattern of patient 102. In connection therewith, speech identification circuitry 502 of signal processing circuitry 454 is configured to identify patient speech data 504 by identifying the at least a portion of the speech data 130 representing the speech pattern that matches a known speech pattern of the patient. Speech pattern matching circuitry 506 may implement methods as discussed herein above to identify the patient, e.g., as described in Chandra, E. and Sunitha, C., “A review on Speech and Speaker Authentication System using Voice Signal feature selection and extraction,” IEEE International Advance Computing Conference, 2009. IACC 2009, Page(s): 1341-1346, 2009 (DOI: 10.1109/IADCC.2009.4809211) or McCowan, I.; Dean, D.; McLaren, M.; Vogt, R.; and Sridharan, S.; “The Delta-Phase Spectrum With Application to Voice Activity Detection and Speaker Recognition,” IEEE Transactions on Audio, Speech, and Language Processing, 2011, Volume: 19, Issue: 7, Page(s): 2026-2038 (DOI: 10.1109/TASL.2011.2109379), both of which are incorporated herein by reference.

In an aspect, identity signal 392 includes an image signal received from an imaging device at the patient location, and patient identification circuitry 452 is configured to analyze the image signal to determine the presence of the patient, using image analysis circuitry 508. In connection therewith, speech identification circuitry 502 is configured to identify patient speech data 504 by identifying at least a portion of the speech data 130 corresponding to an image signal indicative of the patient. In an aspect, patient identification circuitry 452 is configured to analyze the image signal to determine the presence of the patient through facial recognition, using facial recognition circuitry 510. In an aspect, patient identification circuitry 452 is configured to analyze the image signal to determine the presence of the patient through gait recognition, using gait analysis circuitry 512.

In an aspect, identity signal 392 includes a biometric signal from at least one biometric sensor at the patient location (e.g., as described in connection with FIG. 3), and patient identification circuitry 452 is configured to analyze the biometric signal through the use of biometric analysis circuitry 514 to determine the presence of the patient, and speech identification circuitry 502 is configured to identify patient speech data 504 by identifying speech data corresponding to a biometric signal indicative of the patient.

In an aspect, identity signal 392 includes at least one authentication factor, and patient identification circuitry 452 is configured to determine the presence of the patient through the use of authentication circuitry 516. For example, in various aspects authentication factor is a security token, a password, a digital signature, or a cryptographic key.

In an aspect, identity signal 392 includes a cell phone identification code, and patient identification circuitry 452 is configured to determine the presence of the patient through the use of cell phone identification circuitry 518. The cell phone identification code may be, for example, an electronic serial number, a mobile identification number, and a system identification code.

In an aspect, identity signal 392 includes an RFID signal, and patient identification circuitry 452 is configured to determine the presence of the patient through the use of RFID circuitry 520. The RFID may be associated with the communication system or other component of the local system at the patient location, or it may be worn on, carried, or otherwise associated with the patient.

In general, determination of patient presence with patient identification circuitry 452 based on identity signal 392 can be performed using methods as discussed and described in the references cited and incorporated by reference herein above in connection with FIG. 3.

Signal processing circuitry 454 is configured to analyze speech data signal 134 to determine whether speech data 130 is representative of at least one speech pattern 450 that matches at least one characteristic speech pattern 456. In an aspect, the signal processing circuitry 454 includes a speech processor 522.

In an aspect, compliance determination circuitry 458 is configured to determine whether the patient has complied with the prescribed treatment regimen based upon whether speech data 130 represents at least one speech pattern 450 that matches at least one characteristic speech pattern 456. In an aspect, compliance determination circuitry 458 includes speech analyzer 524 for analyzing the speech data to determine the speech pattern 450, and a comparator 526 for comparing the speech pattern represented by the speech data with the at least one characteristic speech pattern 456. In an aspect, comparator 526 is configured to compare speech pattern 450 with a plurality of characteristic speech patterns 456, 540, and 542. In an aspect, comparator 526 is configured for comparing the speech data 130 with characteristic speech data 532 (also referred to as characteristic speech data set 532) representing the characteristic speech pattern. In an aspect, comparator 526 is configured to compare speech data 130 with a plurality of characteristic speech data sets (e.g., characteristic speech data sets 532, 534, and 536), each said characteristic speech data set representing a characteristic speech pattern. In an aspect, compliance determination circuitry 458 is configured to determine whether the patient has complied with the prescribed treatment regimen based upon a determination of whether the received speech data (speech data 130) represents at least one of a plurality of characteristic speech patterns (e.g., characteristic speech pattern 456, 540, and 542). Compliance determination circuitry 458 can be configured to determine that the patient has failed to comply with the prescribed treatment regimen, and/or that the patient has complied with the prescribed treatment regimen.

Reporting circuitry 460 in monitoring system 118 is configured to report a conclusion 140 based on the determination of whether the patient has complied with the prescribed treatment regimen. In various aspects, reporting circuitry 460 includes a display device 544 and notification circuitry 546 for generating a notification. Notification circuitry 546 may include, for example, wireless notification circuitry 548 for transmitting a notification to a wireless device 550, audio alarm circuitry 552 for generating an audio alarm, data storage circuitry 554 for storing a notification in a data storage device 562, or email generation circuitry 558 for generating an email notification.

In an aspect, monitoring system 118 includes at least one input device 556 for receiving prescription information indicative of the treatment regimen prescribed to the patient. Input device 556 can be, for example a user input device 558 (e.g., a keyboard, touchpad, touchscreen, mouse, joystick, microphone or other voice input, etc.) adapted for receiving prescription information from, e.g., medical care provider 126, or data input device 560 adapted to receive data from another device (e.g., a computer system, a networked system, a cell phone, a barcode reader, a flash drive, a disk drive, etc. via a wired or wireless connection as is well known in the relevant arts).

As noted herein above, monitoring system 118 includes at least one data storage device 562. Data stored in data storage device 562 may include, but is not limited to speech data 130, prescription information 564 (including details of the prescribed treatment regimen), stored messages regarding device status, device settings, instructions, or conclusions, for example. Data storage device 562 is a data storage device or system that forms a part of monitoring system 118, or is accessible by monitoring system 118, e.g., on a server and/or cloud-based data storage system. In an aspect, data storage device 562 stores one or more databases containing electronic medical records, for example.

Monitoring system 118 includes at least one receiving device 136, which is used for receiving speech data signal 134 transmitted to monitoring location 120 from patient location 116. In various aspects, receiving device 136 includes a wireless receiver 568, a computer network connection 570, a communication port 572 (e.g., USB port), or a computer drive 574. In an aspect, transmission of data or information to receiving device 136 encompasses wireless or wired transmission. In an aspect, transmission of data or information to receiving device 136 encompasses device-based transmission involving transfer of data from local system 114 at patient location 116, via a data storage device (e.g., a flash drive or DVD), to a data reading device (USB port 572 or computer drive 574) in monitoring system 118 that reads data from the data storage device. Monitoring system 118 in some aspects includes more than one receiving device, and multiple receiving devices may be of the same or different types. In some aspects, receiving device 136 receives various types of data and/or information from local system 114 at patient location 116, not limited to speech data signal 134. In some aspects, receiving device 136 receives identity signal 392 from the patient location. The identity signal 392 may be received from the communication system 106, for example from the transmitting device 132, and may include data processed from sensing devices of the communication system 106. In some aspects, receiving device 136 receives notification 388. In some aspects, receiving device 136 receives treatment signal 394. Furthermore, in some aspects, receiving device 136 receives data or information from devices and systems other than local system 114. For example, the identity signal 392 may be received from sensing devices at the patient location but external to the communication system 106. For example, in some aspects, receiving device 136 may also serve as data input device 560. In various aspects, monitoring system 118 includes one or more receiving devices 136 for receiving identity signal 392, notification 388, treatment signal 394, and/or speech data signal 134.

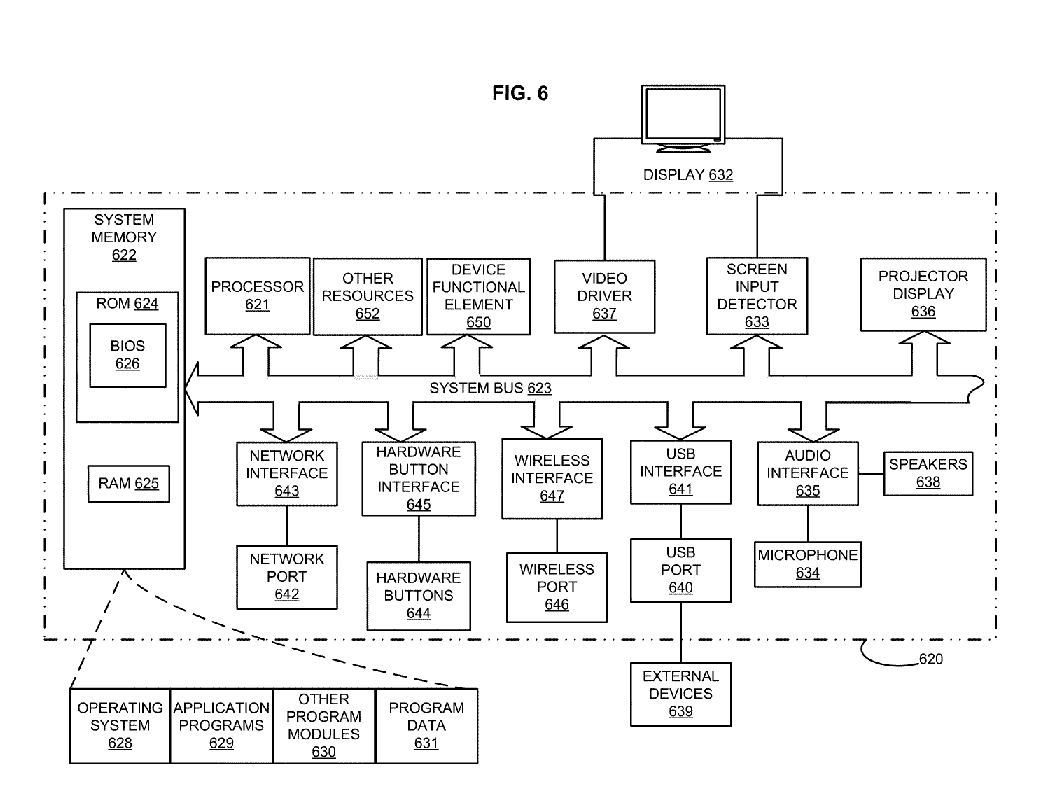

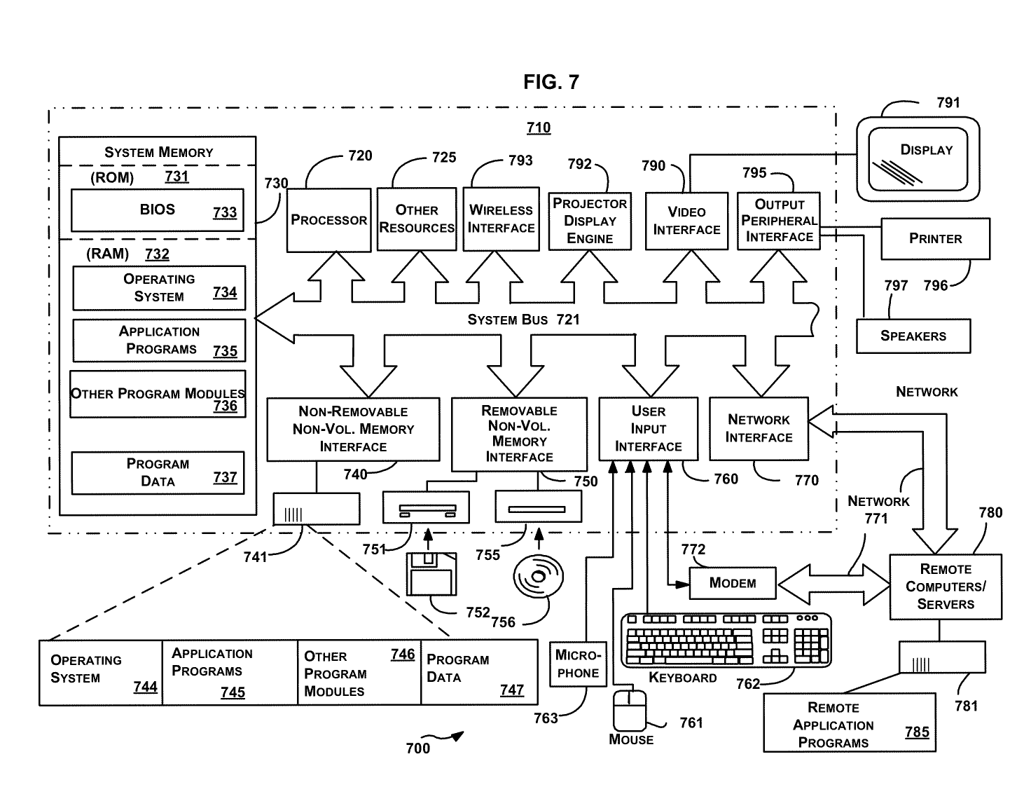

FIGS. 6 and 7 provide brief, general descriptions of environments in which embodiments may be implemented. FIG. 6 illustrates an example system that includes a thin computing device 620, which may be included in an electronic device that also includes one or more device functional element 650. For example, the electronic device may include any item having electrical or electronic components playing a role in a functionality of the item, such as a limited resource computing device, a wireless communication device, a mobile wireless communication device, an electronic pen, a handheld electronic writing device, a digital camera, a scanner, an ultrasound device, an x-ray machine, a non-invasive imaging device, a cell phone, a PDA, a Blackberry® device, a printer, a refrigerator, a car, and an airplane. In another example, the thin computing device may be included in an implantable medical apparatus or device. In a further example, the thin computing device may be operable to communicate with an implantable or implanted medical apparatus.

The thin computing device 620 includes a processor 621, a system memory 622, and a system bus 623 that couples various system components including the system memory 622 to the processor 621. The system bus 623 may be any of several types of bus structures including a memory bus or memory controller, a peripheral bus, and a local bus using any of a variety of bus architectures. In an aspect, the system memory includes read-only memory (ROM) 624 and random access memory (RAM) 625. A basic input/output system (BIOS) 626, containing the basic routines that help to transfer information between sub-components within the thin computing device 620, such as during start-up, is stored in the ROM 624. A number of program modules may be stored in the ROM 624 or RAM 625, including an operating system 628, one or more application programs 629, other program modules 630 and program data 631.

A user may enter commands and information into the computing device 620 through input devices, such as a number of switches and buttons, illustrated as hardware buttons 644, connected to the system via a suitable interface 645. Input devices may further include a touch-sensitive display with suitable input detection circuitry, illustrated as a display 632 and screen input detector 633. The output circuitry of the touch-sensitive display 632 is connected to the system bus 623 via a video driver 637. Other input devices may include a microphone 634 connected through a suitable audio interface 635, and a physical hardware keyboard (not shown). Output devices may include at least one display 632 and at least one speaker 638.

In addition to the display 632, the computing device 620 may include other peripheral output devices, such as a projector display 636. Other external input or output devices 639 may be connected to the processor 621 through a USB port 640 and USB port interface 641, to the system bus 623. Alternatively, the other external input and output devices 639 may be connected by other interfaces, such as a parallel port, game port or other port. External input or output devices 629 include, e.g., a joystick, game pad, satellite dish, scanner, various types of sensors or actuators. Output signals include device control signals. The computing device 620 may further include or be capable of connecting to a flash card memory (not shown) through an appropriate connection port (not shown). The computing device 620 may further include or be capable of connecting with a network through a network port 642 and network interface 643, and through wireless port 646 and corresponding wireless interface 647 may be provided to facilitate communication with other peripheral devices, including other computers, printers, and so on (not shown). It will be appreciated that the various components and connections shown are examples and other components and means of establishing communication links may be used.

The computing device 620 may be primarily designed to include a user interface. The user interface may include a character, a key-based, or another user data input via the touch sensitive display 632. The user interface may include using a stylus (not shown). Moreover, the user interface is not limited to a touch-sensitive panel arranged for directly receiving input, but may alternatively or in addition respond to another input device such as the microphone 634. For example, spoken words may be received at the microphone 634 and recognized. Alternatively, the computing device 620 may be designed to include a user interface having a physical keyboard (not shown).

The device functional elements 650 are typically application specific and related to a function of the electronic device, and is coupled with the system bus 623 through an interface (not shown). The functional elements may typically perform a single well-defined task with little or no user configuration or setup, such as a cell phone connecting with an appropriate tower and transceiving voice or data information, or communicating with an implantable medical apparatus, or a camera capturing and saving an image.

In certain instances, one or more elements of the thin computing device 620 may be deemed not necessary and omitted. In other instances, one or more other elements (e.g., other resources 652) may be deemed necessary and added to the thin computing device.

FIG. 7 illustrates an example embodiment of a computing system in which embodiments may be implemented, shown as a computing system environment 700. Components of the computing system environment 700 may include, but are not limited to, a computing device 710 having a processor 720, a system memory 730, and a system bus 721 that couples various system components including the system memory to the processor 720. The system bus 721 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

The computing system environment 700 typically includes a variety of computer-readable media products. Computer-readable media may include any media that can be accessed by the computing device 710 and include both volatile and nonvolatile media, removable and non-removable media. By way of example, and not of limitation, computer-readable media may include computer storage media. By way of further example, and not of limitation, computer-readable media may include a communication media.

Computer storage media includes volatile and nonvolatile, removable and non-removable media implemented in any method or technology for storage of information such as computer-readable instructions, data structures, program modules, or other data. Computer storage media includes, but is not limited to, random-access memory (RAM), read-only memory (ROM), electrically erasable programmable read-only memory (EEPROM), flash memory, or other memory technology, CD-ROM, digital versatile disks (DVD), or other optical disk storage, magnetic cassettes, magnetic tape, magnetic disk storage, or other magnetic storage devices, or any other medium which can be used to store the desired information and which can be accessed by the computing device 710. In a further embodiment, a computer storage media may include a group of computer storage media devices. In another embodiment, a computer storage media may include an information store. In another embodiment, an information store may include a quantum memory, a photonic quantum memory, or atomic quantum memory. Combinations of any of the above may also be included within the scope of computer-readable media.

Communication media may typically embody computer-readable instructions, data structures, program modules, or other data in a modulated data signal such as a carrier wave or other transport mechanism and include any information delivery media. The term “modulated data signal” means a signal that has one or more of its characteristics set or changed in such a manner as to encode information in the signal. By way of example, and not limitation, communication media include wired media, such as a wired network and a direct-wired connection, and wireless media such as acoustic, RF, optical, and infrared media.

The system memory 730 includes computer storage media in the form of volatile and nonvolatile memory such as ROM 731 and RAM 732. A RAM may include at least one of a DRAM, an EDO DRAM, a SDRAM, a RDRAM, a VRAM, or a DDR DRAM. A basic input/output system (BIOS) 733, containing the basic routines that help to transfer information between elements within the computing device 710, such as during start-up, is typically stored in ROM 731. RAM 732 typically contains data and program modules that are immediately accessible to or presently being operated on by processor 720. By way of example, and not limitation, FIG. 7 illustrates an operating system 734, application programs 735, other program modules 736, and program data 737. Often, the operating system 734 offers services to applications programs 735 by way of one or more application programming interfaces (APIs) (not shown). Because the operating system 734 incorporates these services, developers of applications programs 735 need not redevelop code to use the services. Examples of APIs provided by operating systems such as Microsoft's “WINDOWS” are well known in the art.

The computing device 710 may also include other removable/non-removable, volatile/nonvolatile computer storage media products. By way of example only, FIG. 7 illustrates a non-removable non-volatile memory interface (hard disk interface) 740 that reads from and writes for example to non-removable, non-volatile magnetic media. FIG. 7 also illustrates a removable non-volatile memory interface 750 that, for example, is coupled to a magnetic disk drive 751 that reads from and writes to a removable, non-volatile magnetic disk 752, or is coupled to an optical disk drive 755 that reads from and writes to a removable, non-volatile optical disk 756, such as a CD ROM. Other removable/nonremovable, volatile/non-volatile computer storage media that can be used in the example operating environment include, but are not limited to, magnetic tape cassettes, memory cards, flash memory cards, DVDs, digital video tape, solid state RAM, and solid state ROM. The hard disk drive 741 is typically connected to the system bus 721 through a non-removable memory interface, such as the interface 740, and magnetic disk drive 751 and optical disk drive 755 are typically connected to the system bus 721 by a removable non-volatile memory interface, such as interface 750.

The drives and their associated computer storage media discussed above and illustrated in FIG. 7 provide storage of computer-readable instructions, data structures, program modules, and other data for the computing device 710. In FIG. 7, for example, hard disk drive 741 is illustrated as storing an operating system 744, application programs 745, other program modules 746, and program data 747. Note that these components can either be the same as or different from the operating system 734, application programs 735, other program modules 736, and program data 737. The operating system 744, application programs 745, other program modules 746, and program data 747 are given different numbers here to illustrate that, at a minimum, they are different copies.

A user may enter commands and information into the computing device 710 through input devices such as a microphone 763, keyboard 762, and pointing device 761, commonly referred to as a mouse, trackball, or touch pad. Other input devices (not shown) may include at least one of a touch sensitive display, joystick, game pad, satellite dish, and scanner. These and other input devices are often connected to the processor 720 through a user input interface 760 that is coupled to the system bus, but may be connected by other interface and bus structures, such as a parallel port, game port, or a universal serial bus (USB). Other devices that can be coupled to the system bus via other interface and bus structures include sensors of various types, for example.

A display 791, such as a monitor or other type of display device or surface may be connected to the system bus 721 via an interface, such as a video interface 790. A projector display engine 792 that includes a projecting element may be coupled to the system bus. In addition to the display, the computing device 710 may also include other peripheral output devices such as speakers 797 and printer 796, which may be connected through an output peripheral interface 795. Outputs may be sent to a variety of other types of devices, and are not limited to the example output devices identified here.

The computing system environment 700 may operate in a networked environment using logical connections to one or more remote computers, such as a remote computer 780. The remote computer 780 may be a personal computer, a server, a router, a network PC, a peer device, or other common network node, and typically includes many or all of the elements described above relative to the computing device 710, although only a memory storage device 781 has been illustrated in FIG. 7. The network logical connections depicted in FIG. 7 include a local area network (LAN) and a wide area network (WAN), and may also include other networks such as a personal area network (PAN) (not shown). Such networking environments are commonplace in offices, enterprise-wide computer networks, intranets, and the Internet.

When used in a networking environment, the computing system environment 700 is connected to the network 771 through a network interface, such as the network interface 770, the modem 772, or the wireless interface 793. The network may include a LAN network environment, or a WAN network environment, such as the Internet. In a networked environment, program modules depicted relative to the computing device 710, or portions thereof, may be stored in a remote memory storage device. By way of example, and not limitation, FIG. 7 illustrates remote application programs 785 as residing on computer medium 781. It will be appreciated that the network connections shown are examples and other means of establishing a communication link between the computers may be used.

In certain instances, one or more elements of the computing device 710 may be deemed not necessary and omitted. In other instances, one or more other elements (e.g., other resources 725) may be deemed necessary and added to the computing device.

FIGS. 6 and 7 illustrate generalized forms of circuitry-based systems, in which systems as depicted in FIGS. 1-5 may be implemented. Although specific embodiments are described herein, those skilled in the art will appreciate that methods and systems as described herein can be implemented in various ways. Reference is made herein to various circuitry systems/subsystems (e.g., patient identification circuitry 202, speech detection circuitry 206, notification circuitry 390 in FIG. 3, and patient identification circuitry 452, reporting circuitry 460, and signal processing circuitry 454 in FIG. 5) which may be considered to be control/processing circuitry, and/or components thereof. In general, control/processing circuitry (e.g., control/processing circuitry 128 and control/processing circuitry 138 in FIG. 1) includes any or all of digital and/or analog components, one or more processor (e.g., a microprocessor), and includes memory and additional components as described in connection with FIGS. 6 and 7.

In a general sense, those skilled in the art will recognize that the various embodiments described herein can be implemented, individually and/or collectively, by various types of electrical circuitry having a wide range of electrical components such as hardware, software, firmware, and/or virtually any combination thereof. Electrical circuitry includes electrical circuitry having at least one discrete electrical circuit, electrical circuitry having at least one integrated circuit, electrical circuitry having at least one application specific integrated circuit, electrical circuitry forming a computing device configured by a computer program (e.g., a computer configured by a computer program which at least partially carries out processes and/or devices described herein, or a microprocessor configured by a computer program which at least partially carries out processes and/or devices described herein), electrical circuitry forming a memory device, which may include various types of memory (e.g., random access, flash, read only, etc.), electrical circuitry forming a communications device (e.g., a modem, communications switch, optical-electrical equipment, etc.), and/or any non-electrical analog thereto, such as optical or other analogs (e.g., graphene based circuitry). In an embodiment, the system is integrated in such a manner that the system operates as a unique system configured specifically for the function of monitoring treatment compliance, and any associated computing devices of the system operate as specific use computers for purposes of the claimed system, and not general use computers. In an embodiment, at least one of the associated computing devices of the system is hardwired with a specific ROM to instruct the at least one computing device. In a general sense, those skilled in the art will recognize that the various aspects described herein which can be implemented, individually and/or collectively, by a wide range of hardware, software, firmware, and/or any combination thereof can be viewed as being composed of various types of “electrical circuitry.”

At least a portion of the devices and/or processes described herein can be integrated into a data processing system. A data processing system generally includes one or more of a system unit housing, a video display, memory such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), and/or control systems including feedback loops and control motors (e.g., feedback for sensing position and/or velocity; control motors for moving and/or adjusting components and/or quantities). A data processing system may be implemented utilizing suitable commercially available components, such as those typically found in data computing/communication and/or network computing/communication systems.

As discussed in connection with FIG. 1, transmitting device 132 in local system 114 and receiving device 136 in monitoring system 118 are configured to provide a communication link between the two locations. In various aspects, transmitting device 132 and receiving device 136 provide a wireless communication link. A wireless communication link may also be established between monitoring system 118 and wireless device 550, as shown in FIG. 5. In various aspects, a wireless communication link includes at least one of a radio frequency, wireless network, cellular network, satellite, WiFi, BlueTooth, Wide Area Network (WAN), Local Area Network (LAN), or Body Area Network (BAN) communication link. Various types of communication links are suitable for providing communication between two remote locations. Communication between locations remote from each other may take place over telecommunications networks, for example public or private Wide Area Network (WAN). In general, communication between remote locations is not considered to be suitably handled by technologies geared towards physically localized networks, e.g., Local Area Network (LAN) technologies operation at Layer 1/2 (such as the forms of Ethernet or WiFi). However, it will be appreciated that portions (but not the entirety) of communication networks used in remote communications may include technologies suitable for use in physically localized network, such as Ethernet or WiFi. In an aspect, system components are considered “remote” from each other if they are not within the same room, building, or campus. In an aspect, a remote system may include components separated by a few miles or more. Conversely, system components may be considered “local” to each other if they are located within the same room, building, or campus.

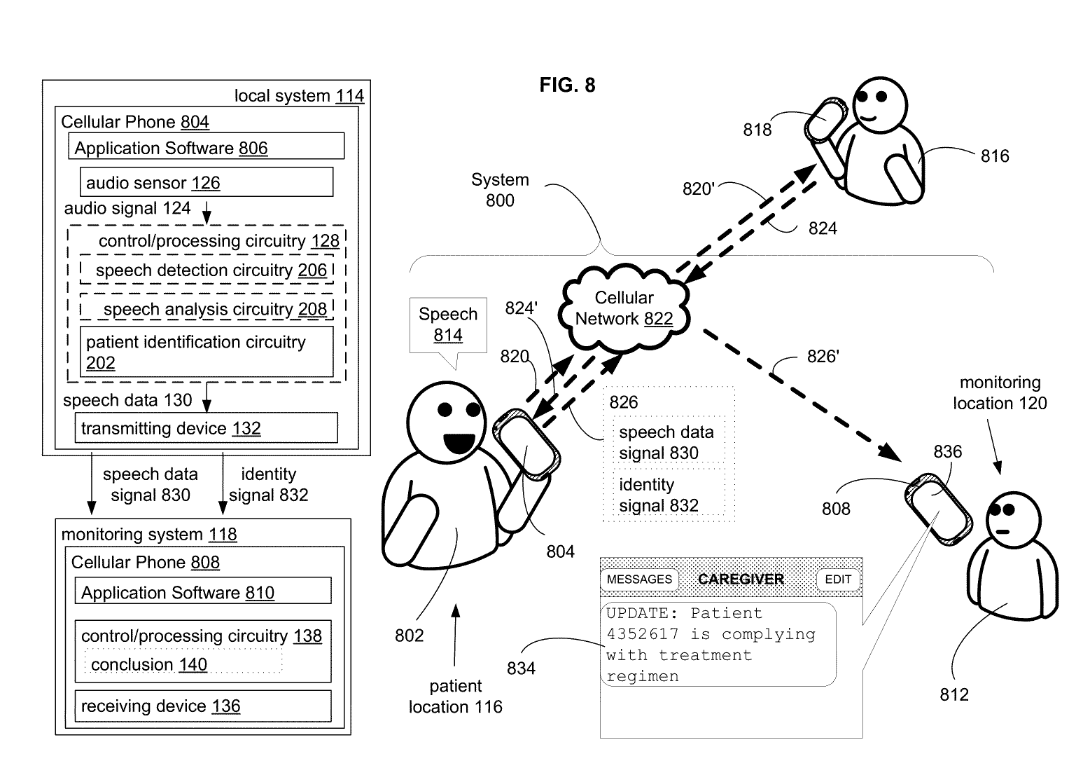

FIG. 8 is an illustration of a system 800 for monitoring compliance of a patient 802 with a treatment regimen, implemented in a cell phone 804. System 800 includes cell phone 804, which is a cell phone used by patient 802 and configured with application software 806, and cell phone 808, which is configured with application software 810 and used by medical care provider 812. In the example of FIG. 8, patient compliance with a treatment regimen is monitored, and a report is provided to medical care provider 812, while patient 802 uses cell phone 804 for personal communications. Cell phone 804 and application software 806 serve as local system 114 at patient location 116, while cell phone 808 and application software 810 serve as monitoring system 118 at monitoring location 120. System 800 is used to monitor compliance of patient 802 with a prescribed treatment regimen by analyzing speech 814 of patient 802 during the course of routine use of cell phone 804 by patient 802, for example to communicate with person 816 (e.g., a friend), who is using a cell phone 818. During communication with person 816, a conventional cellular communication signal 820, containing voice data from patient 802 is transmitted to cellular network 822 and from there to cell phone 818 as signal 820′. Similarly, cellular communication signal 824, 824′ containing voice data from person 816 is transmitted from cell phone 818 to cell phone 804 via cellular network 822. A second cellular signal 826, 826′ is transmitted via cellular network 822 to cell phone 808. Second cellular signal 826, 826′ contains speech data signal 830 and identity signal 832, which are processed by control/processing circuitry 138 on phone 808 (including application software 810 on cell phone 808 as well as telephone system hardware/software) to generate report 834. Speech data signal 830 contains speech parameters that characterize the speech of patient 802, but does not contain the speech itself, thereby maintaining the privacy of patient 802's communications. Furthermore, speech data signal 830 does not contain speech from person 816. Processing of speech data signal 830 occurs on cell phone 804, through the use of application software 806 as well as phone system hardware/software, functioning as control/processing circuitry 128, speech detection circuitry 206, speech analysis circuitry 208, and patient identification circuitry 202 as described elsewhere herein. Speech data signal 830 and identity signal 832 are transmitted from transmitting device 132 in phone 804 to receiving device 136 in phone 808, where processing is performed by control/processing circuitry 138 as described herein above. As depicted in FIG. 8, report 834 including a conclusion 140 is presented to medical care provider 812 in the form of a text message displayed on screen 836 of cell phone 808. In the example of FIG. 8, the conclusion is that “Patient 4352617 is complying with treatment regimen.”

FIG. 9 is an illustration a compliance monitoring system 900 including local system 114, which in this example is a computing system 902 at patient location 116, and monitoring system 118, which is a computing system 904 at monitoring location 120. In the example of FIG. 9, in a first monitoring mode, patient 906 participates in a video consultation with medical care provider 908, with patient voice data being captured by microphone 910, patient image data being captured by camera 912, and both voice and image data being transmitted to computer 914 of computing system 904 via network 916. An image 918 of patient 906 is displayed on display 920 for viewing by medical care provider 908. Camera 922 captures an image 924 of medical care provider 908, which is transmitted to system 902 via network 916, where it is displayed on display 926. Microphone 930 captures voice data from medical care provider 908, which is also sent to system 902 and may be delivered to patient 906 via speakers 932. Similarly, patient voice data can be presented to medical care provider 908 via speakers 934. In addition to patient image 918, a report 936 containing a conclusion 938 regarding compliance of patient 906 with a prescribed treatment regimen is displayed on display 920. In the example of FIG. 9, report 936 includes a listing of a patient ID number, a date, a time, and a statement regarding patient compliance, e.g., “Patient speech parameters indicate partial compliance with prescribed treatment regimen.” Patient identity is determined by entry of an authentication factor 940 (e.g., login and password) by patient 906 when logging in for video conference. Computing system 902 incorporates control/processing circuitry 128, including speech detection circuitry 206, speech analysis circuitry 208, and patient identification circuitry 202, which are as described herein above. In this example, speech data signal 942 includes patient speech 952 (used for audio communication with caregiver 908 as well as for assessing patient compliance with a treatment regimen). Identity signal 944 includes authentication factor 940, as entered by the user (or, alternatively, a value determined with based on authentication factor 940, such as a patient identification number).

In a second monitoring mode, which is used as the patient is working on computing system 902 or in the vicinity, but is not necessarily engaged in a video conference with medical care provider 908, data streaming device 950 captures speech 952 from patient 906 with a built-in microphone and provides for transmission of speech data to network 916. Patient identity is determined by voice recognition. In this case, speech data signal 942 may include minimally processed speech data (e.g., speech data which has been processed to remove non-speech intervals) while identity signal 944 includes speech parameters sufficient to determine patient identity (e.g., speech frequency composition). Patient speech data is transmitted from data streaming device 950 to receiving device 136 of monitoring system 118 via network 916, for further processing by control/processing circuitry 138 of computing system 904 and reporting of conclusion 938 to medical care provider 908.

FIG. 10 is a flow diagram of a method 1000 relating to monitoring compliance of a patient with a prescribed treatment regimen. Method 1000 includes receiving a speech data signal with a receiving device at a monitoring location, the speech data signal transmitted to the monitoring location from a patient location, the speech data signal containing speech data representing at least one speech pattern in speech sensed from a patient by at least one audio sensor of a communication system at the patient location during use of the communication system by the patient, the patient having a brain-related disorder and a prescribed treatment regimen for treating at least one aspect of the brain-related disorder, as indicated at 1002; determining a presence of the patient with patient identification circuitry at the monitoring location from at least one identity signal received at the monitoring location from the patient location, as indicated at 1004; analyzing the speech data signal with signal processing circuitry at the monitoring location to determine whether the speech data represents at least one speech pattern that matches at least one characteristic speech pattern, as indicated at 1006; determining with compliance determination circuitry whether the patient has complied with the prescribed treatment regimen based on whether the speech data represents the at least one speech pattern that matches the at least one characteristic speech pattern, as indicated at 1008; and reporting with reporting circuitry a conclusion based on the determination of whether the patient has complied with the prescribed treatment regimen, as indicated at 1010. In various aspects, method 1000 is carried out with monitoring system 118 as depicted in FIGS. 4 and 5, for example.

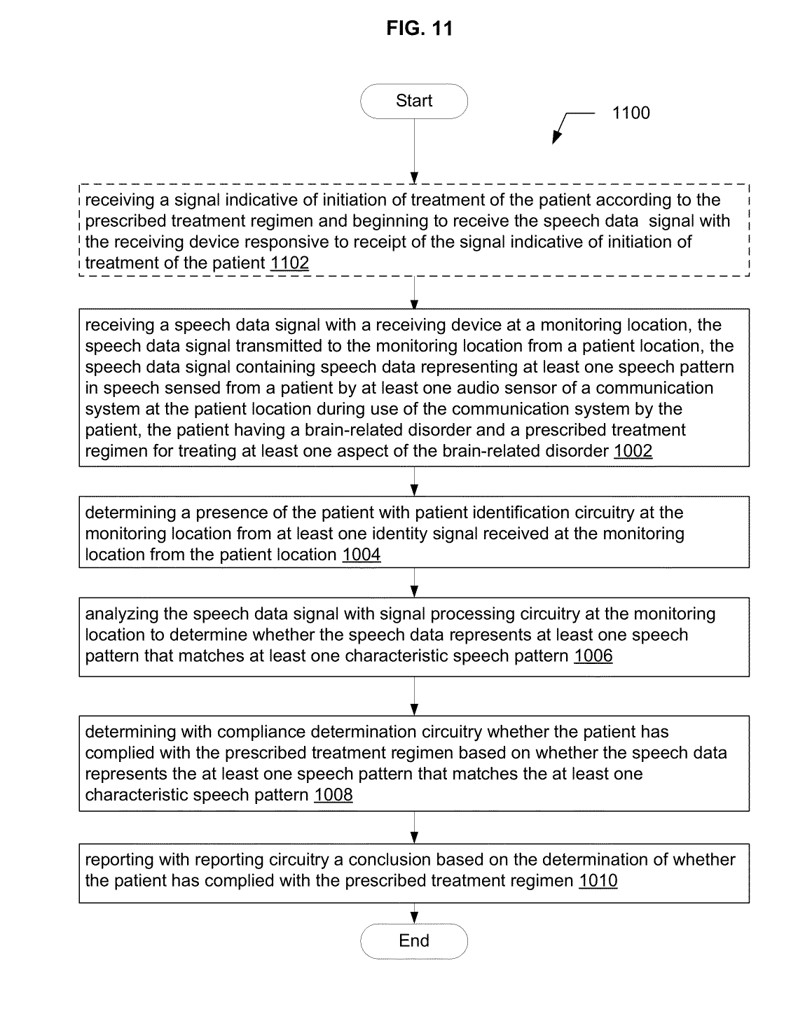

FIGS. 11-22 depict variations and expansions of method 1000 as shown in FIG. 10. In the methods depicted in FIGS. 11-22, steps 1002-1010 are as described generally in connection with FIG. 10. Here and elsewhere, method steps outlined with dashed lines represent steps that are included in some, but not all method aspects, and combinations of steps other than those specifically depicted in the figures are possible as would be known by those having ordinary skill in the relevant art.

FIG. 11 depicts method 1100, which includes steps 1002-1010 as described above, and also includes receiving a signal indicative of initiation of treatment of the patient according to the prescribed treatment regimen, and beginning to receive the speech data signal with the receiving device responsive to receipt of the signal indicative of initiation of treatment of the patient, as indicated at 1102. In some aspects, patient 102 provides an input via a user input device such as user input device 330 in FIG. 3 (e.g., a keyboard or keypad) to indicate that treatment has been initiated (e.g., that the patient took a dose of medication), and treatment signal 412 is transmitted from communication system 106 to the monitoring location. See, e.g., treatment signal 412 received by monitoring system 118 in FIG. 5. In other aspects, treatment signal 580 indicating that treatment has been initiated is received by input device 556 from medical care provider 126 or other individual or entity.

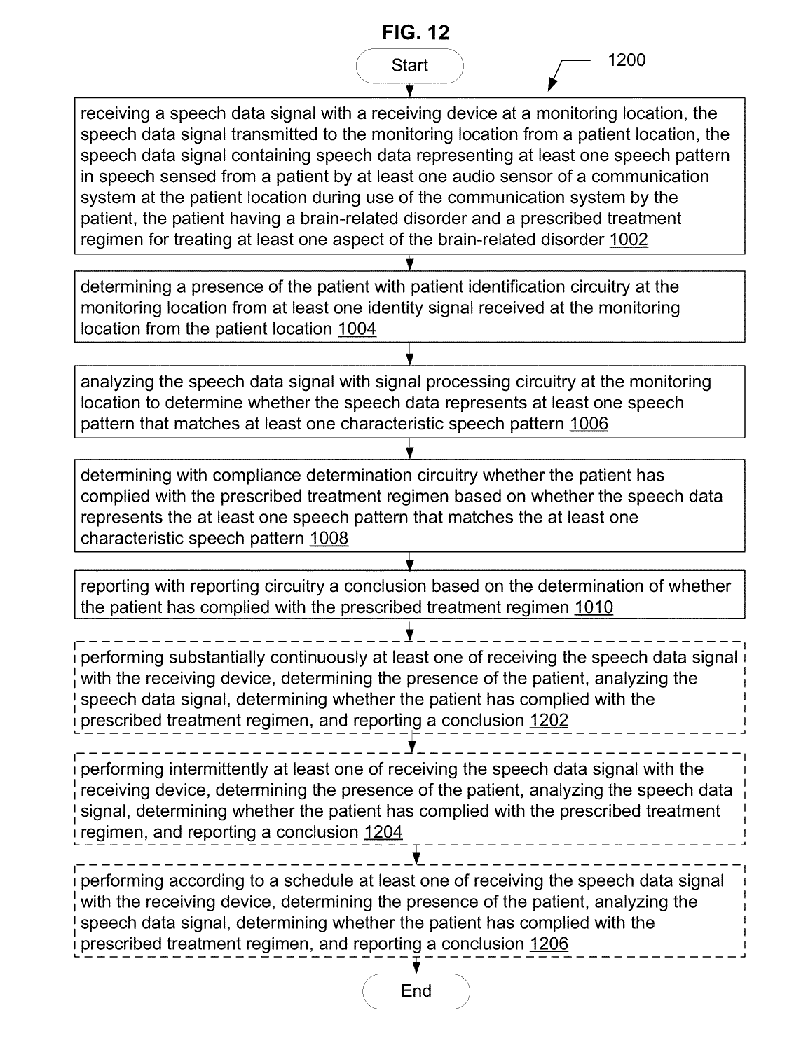

FIG. 12 depicts a method 1200, which includes performing substantially continuously at least one of receiving the speech data signal with the receiving device, determining the presence of the patient, analyzing the speech data signal, determining whether the patient has complied with the prescribed treatment regimen, and reporting a conclusion, as indicated at 1202. In an aspect, method 1200 includes performing intermittently at least one of receiving the speech data signal with the receiving device, determining the presence of the patient, analyzing the speech data signal, determining whether the patient has complied with the prescribed treatment regimen, and reporting a conclusion, as indicated at 1204. In another aspect, method 1200 includes performing according to a schedule at least one of receiving the speech data signal with the receiving device, determining the presence of the patient, analyzing the speech data signal, determining whether the patient has complied with the prescribed treatment regimen, and reporting a conclusion, as indicated at 1206.

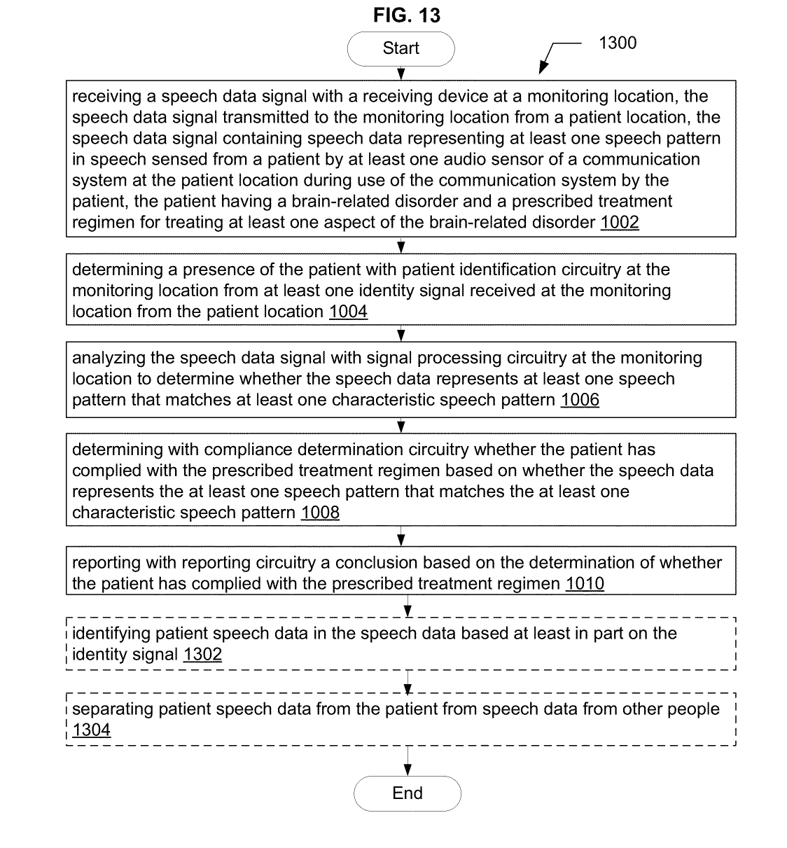

As shown in FIG. 13, a method 1300 includes identifying patient speech data in the speech data based at least in part on the identity signal, as indicated at 1302. In another aspect, method 1300 includes separating patient speech data from the patient from speech data from other people, as indicated at 1304.

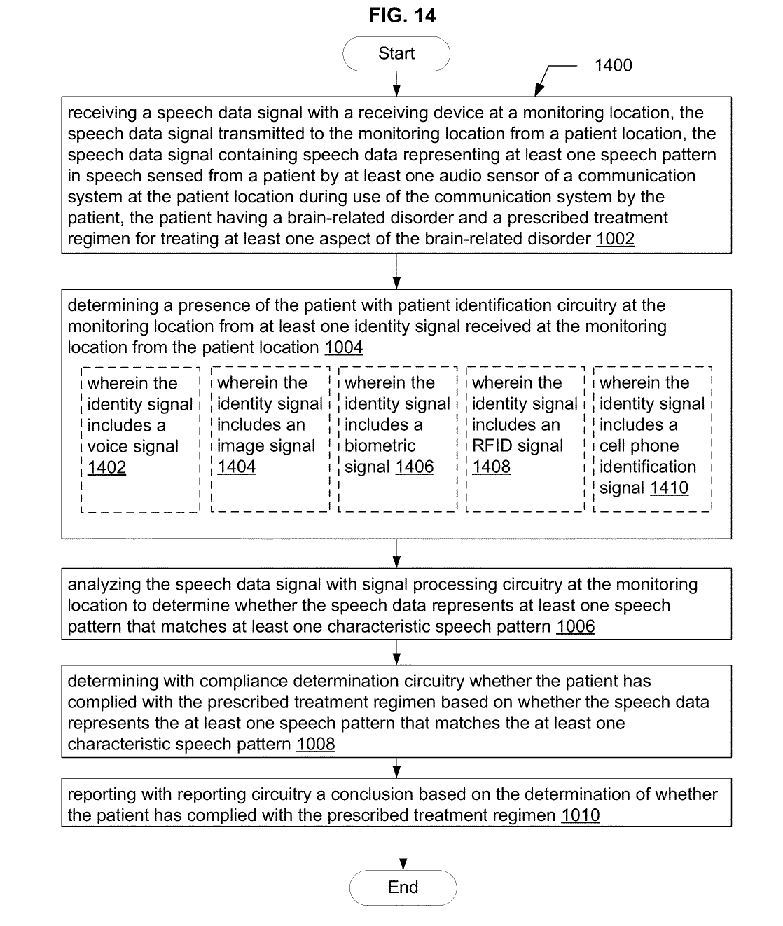

As shown in FIG. 14, in various aspects of a method 1400, the identity signal includes at least one of a voice signal, as indicated at 1402; an image signal, as indicated at 1404; a biometric signal, as indicated at 1406; an RFID signal, as indicated at 1408; or a cell phone identification signal, as indicated at 1410.

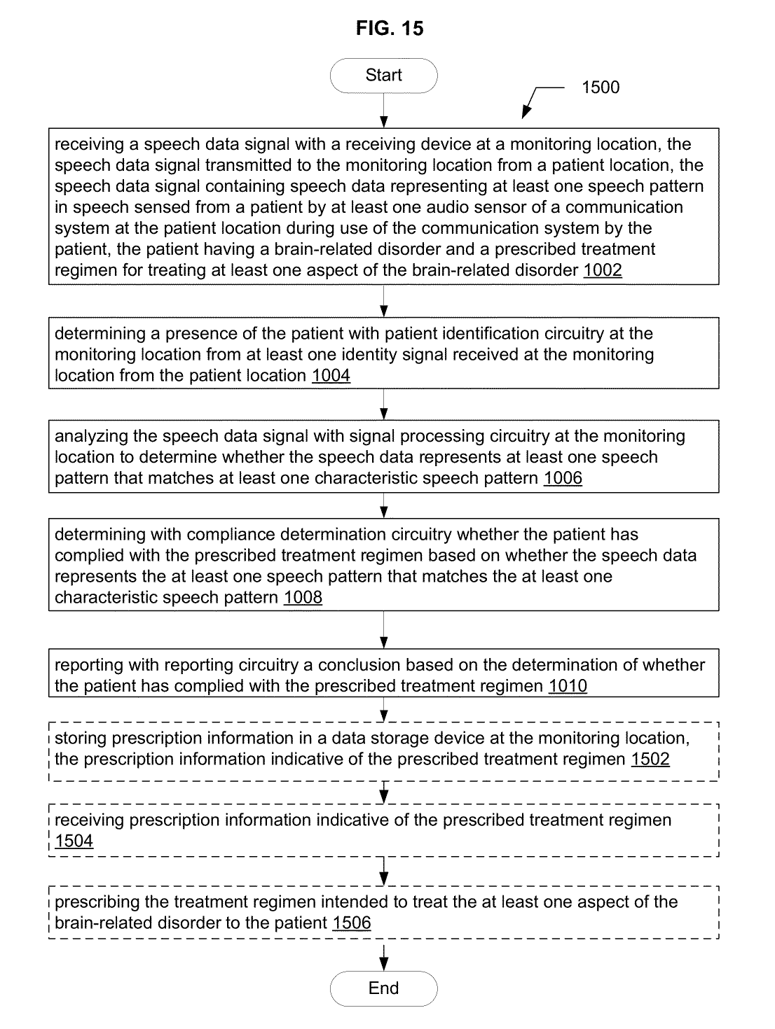

As shown in FIG. 15, in various aspects method 1500 includes at least one of storing prescription information in a data storage device at the monitoring location, the prescription information indicative of the prescribed treatment regimen, as indicated at 1502; receiving prescription information indicative of the prescribed treatment regimen, as indicated at 1504; or prescribing the treatment regimen intended to treat the at least one aspect of a the brain-related disorder to the patient, as indicated at 1506.

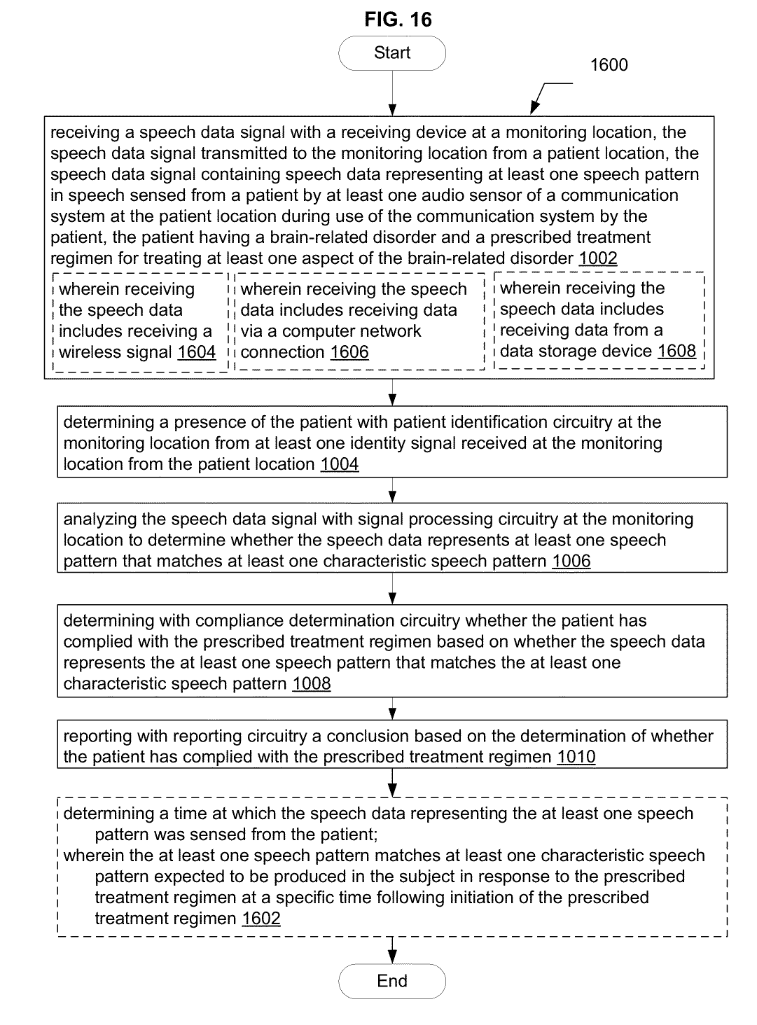

As shown in FIG. 16, in an aspect a method 1600 includes determining a time at which the speech data representing the at least one speech pattern was sensed from the patient, wherein the at least one speech pattern matches at least one characteristic speech pattern expected to be produced in the subject in response to the prescribed treatment regimen at a specific time following initiation of the prescribed treatment regimen, as indicated at 1602. In various aspects, receiving the speech data signal includes one or more of receiving a wireless signal, as indicated at 1604; receiving data via a computer network connection, as indicated at 1606; or receiving data from a data storage device, as indicated at 1608.

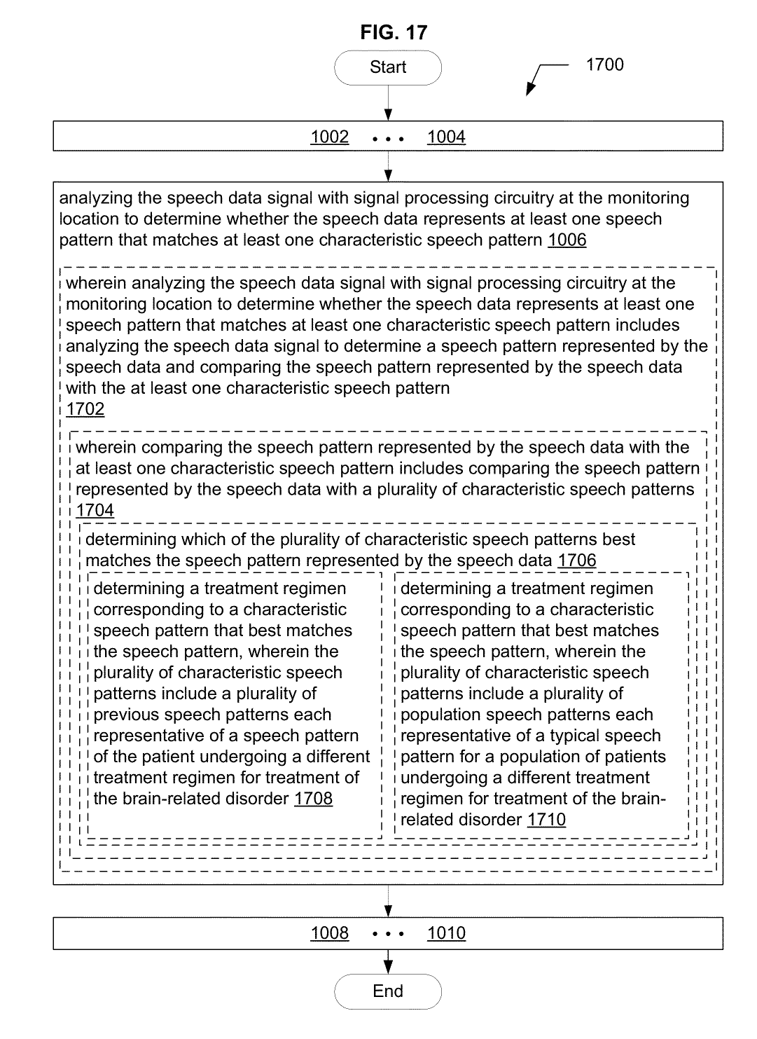

FIG. 17 depicts a method 1700, which includes steps 1002-1010 as described herein above, wherein analyzing the speech data signal with signal processing circuitry at the monitoring location to determine whether the speech data represents at least one speech pattern that matches at least one characteristic speech pattern includes analyzing the speech data signal to determine a speech pattern represented by the speech data, and comparing the speech pattern represented by the speech data with the at least one characteristic speech pattern, as indicated at 1702. In a further aspect, as indicated at 1704, comparing the speech pattern represented by the speech data with the at least one characteristic speech pattern includes comparing the speech pattern represented by the speech data with a plurality of characteristic speech patterns. For example, in an aspect, method 1700 includes determining which of the plurality of characteristic speech patterns best matches the speech pattern represented by the speech data, as indicated at 1706. In an aspect, determining a treatment regimen corresponding to a characteristic speech pattern that best matches the speech pattern, wherein the plurality of characteristic speech patterns include a plurality of previous speech patterns each representative of a speech pattern of the patient undergoing a different treatment regimen for treatment of the brain-related disorder, as indicated at 1708. In another aspect, determining a treatment regimen corresponding to a characteristic speech pattern that best matches the speech pattern, wherein the plurality of characteristic speech patterns include a plurality of population speech patterns each representative of a typical speech pattern for a population of patients undergoing a different treatment regimen for treatment of the brain-related disorder, as indicated at 1710.

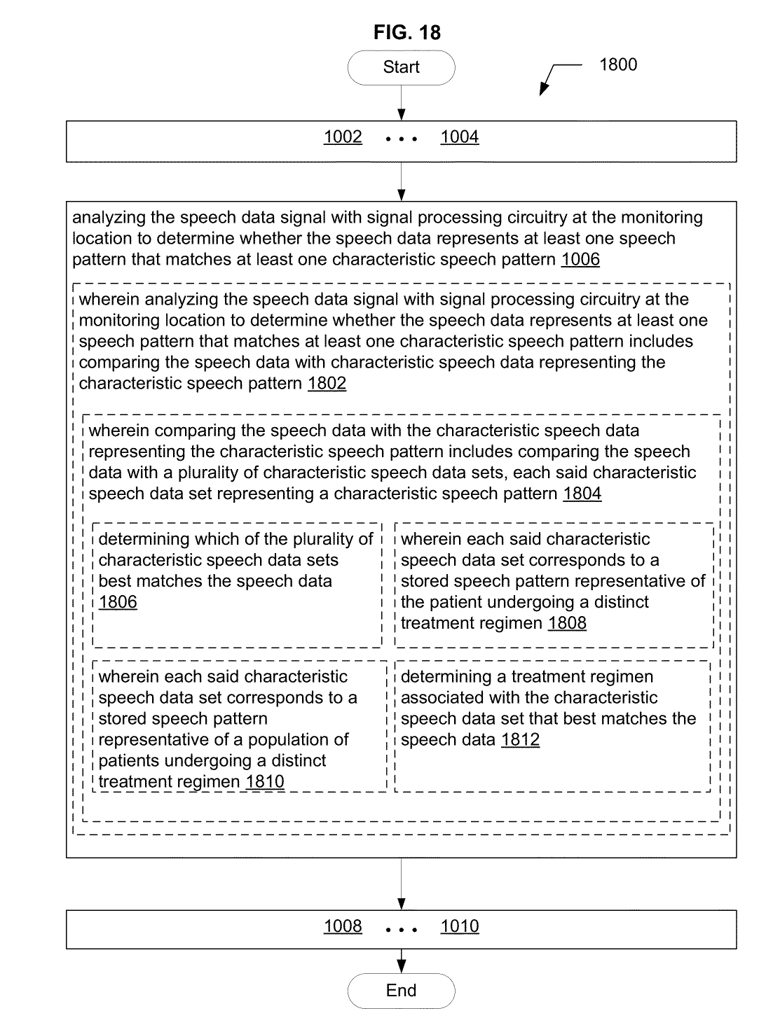

FIG. 18 depicts a method 1800, which includes steps 1002-1010 as described herein above, wherein analyzing the speech data signal with signal processing circuitry at the monitoring location to determine whether the speech data represents the at least one speech pattern that matches at least one characteristic speech pattern includes comparing the speech data with characteristic speech data (a characteristic speech data set) representing the characteristic speech pattern, as indicated at 1802. In an aspect, comparing the speech data with the characteristic speech data representing the characteristic speech pattern includes comparing the speech data with a plurality of characteristic speech data sets, each said characteristic speech data set representing a characteristic speech pattern, as indicated at 1804. In an aspect, method 1800 includes determining which of the plurality of characteristic speech data sets best matches the speech data, as indicated at 1806. In an aspect, each said characteristic speech data set corresponds to a stored speech pattern representative of the patient undergoing a distinct treatment regimen, as indicated at 1808. In an aspect, each said characteristic speech data set corresponds to a stored speech pattern representative of a population of patients undergoing a distinct treatment regimen, as indicated at 1810. In an aspect, determining a treatment regimen associated with the characteristic speech data set that best matches the speech data, as indicated at 1812.

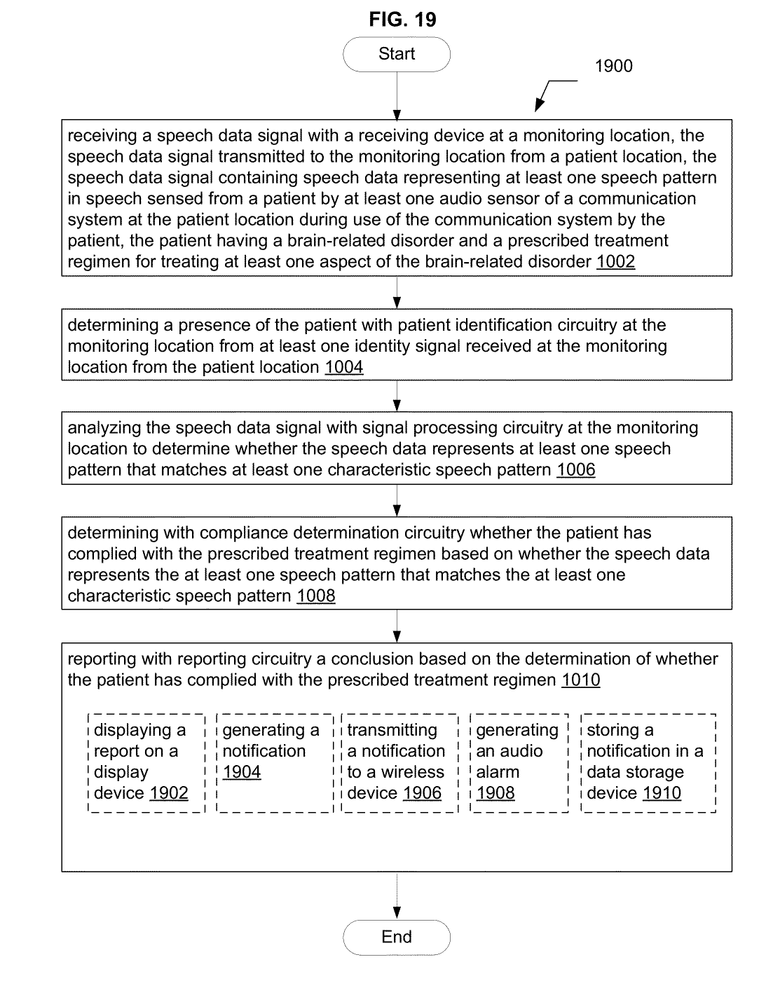

As shown in FIG. 19, in various aspects of a method 1900 reporting a conclusion based on the determination of whether the patient has complied with the prescribed treatment regimen includes at least one of displaying a report on a display device, as indicated at 1902; generating a notification, as indicated at 1904; transmitting a notification to a wireless device, as indicated at 1906; generating an audio alarm, as indicated at 1908; and storing a notification in a data storage device, as indicated at 1910.

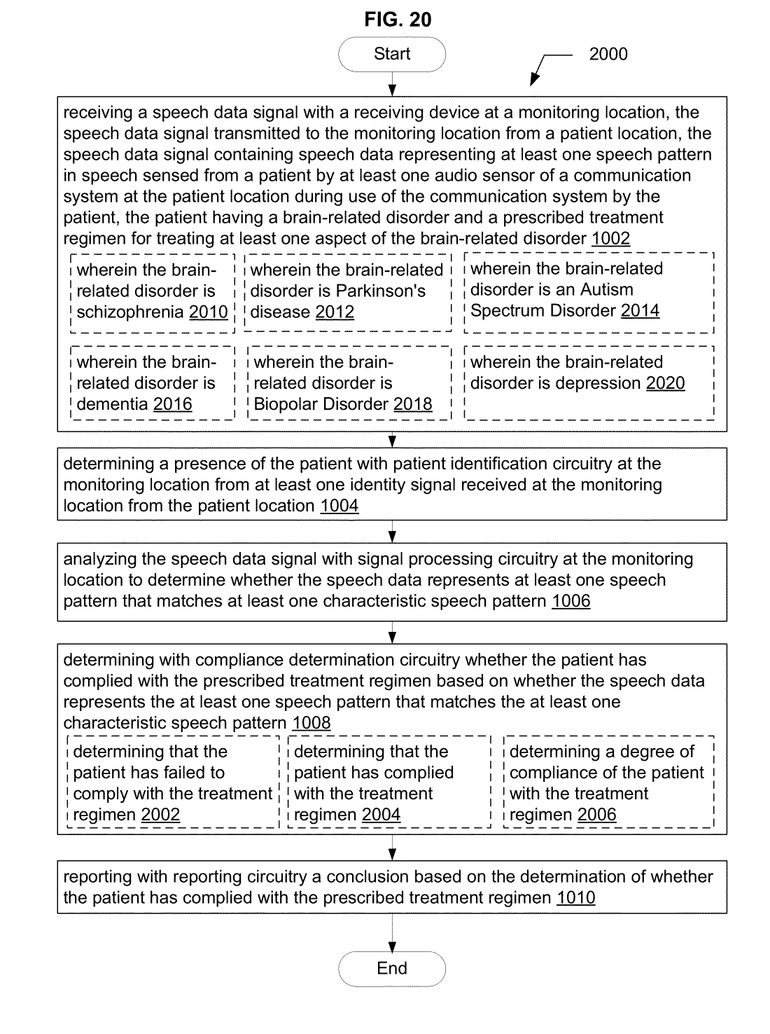

As shown in FIG. 20, in aspects of method 2000, determining with compliance determination circuitry whether the patient has complied with the prescribed treatment regimen includes at least one of determining that the patient has failed to comply with the prescribed treatment regimen, as indicated at 2002; determining that the patient has complied with the prescribed treatment regimen, as indicated at 2004; or determining a degree of compliance of the patient with the prescribed treatment regimen, as indicated at 2006. In various aspects of this and other methods described herein, the brain-related disorder includes at least one of schizophrenia, as indicated at 2010; Parkinson's disease, as indicated at 2012; an Autism Spectrum Disorder, as indicated at 2014; dementia, as indicated at 2016; Biopolar Disorder, as indicated at 2018; or depression, as indicated at 2020.

In an aspect, a brain-related disorder is a mental disorder, psychological disorder, or psychiatric disorder. A mental disorder, psychological disorder, or psychiatric disorder can include, for example, a psychological pathology, psychopathology, psychosocial pathology, social pathology, or psychobiology disorder. A mental disorder, psychological disorder, or psychiatric disorder can be any disorder categorized in any Diagnostic and Statistical Manual (DSM) or International Statistical Classification of Diseases (ICD) Classification of Mental and Behavioural Disorders text, and may be, for example and without limitation, a neurodevelopmental disorder (e.g., autism spectrum disorder or attention-deficit/hyperactivity disorder), a psychotic disorder (e.g., schizophrenia), a mood disorder, a bipolar disorder, a depressive disorder, an anxiety disorder, an obsessive-compulsive disorder, a trauma- or stressor-related disorder, a dissociative disorder, a somatic symptom disorder, an eating disorder, an impulse-control disorder, a substance-related or addictive disorder, a personality disorder (e.g., narcissistic personality disorder or antisocial personality disorder), a neurocognitive disorder, a major or mild neurocognitive disorder (e.g., one due to Alzheimer's disease, traumatic brain injury, HIV infection, prion disease, Parkinson's disease, Huntington's disease, or substance/medication). A mental disorder, psychological disorder, or psychiatric disorder can be any disorder described by the NIH National Institute of Mental Health (NIMH) Research Domain Criteria Project and may include a biological disorder involving brain circuits that implicate specific domains of cognition, emotion, or behavior. In an aspect, a brain-related disorder includes a serious mental illness or serious emotional disturbance.

In various aspects, a brain-related disorder includes a serious mental illness or serious emotional disturbance, a mental disorder, psychological disorder, or psychiatric disorder.

In an aspect, a brain disorder is a traumatic disorder, such as a traumatic brain injury. Traumatic brain injury-induced disorders may present with dysfunction in cognition, communication, behavior, depression, anxiety, personality changes, aggression, acting out, or social inappropriateness. See, e.g., Jeffrey Nicholl and W. Curt LaFrance, Jr., “Neuropsychiatric Sequelae of Traumatic Brain Injury,” Semin Neurol. 2009, 29(3):247-255.

In an aspect, a brain-related disorder is a lesion-related disorder. A brain lesion can include, for example and without limitation, a tumor, an aneurysm, ischemic damage (e.g., from stroke), an abscess, a malformation, inflammation, or any damage due to trauma, disease, or infection. An example of a lesion-related disorder is a disorder associated with a right-hemisphere lesion.

In an aspect a brain disorder is a neurological disorder. A neurological disorder may be, for example and without limitation, Alzheimer's disease, a brain tumor, a developmental disorder, epilepsy, a neurogenetic disorder, Parkinson's disease, Huntington's disease, a neurodegenerative disorder, stroke, traumatic brain injury or a neurological consequence of AIDS. Neurological disorders are described on the website of the National Institutes of Health (NIH) National Institute of Neurological Disorders and Stroke (NINDS).

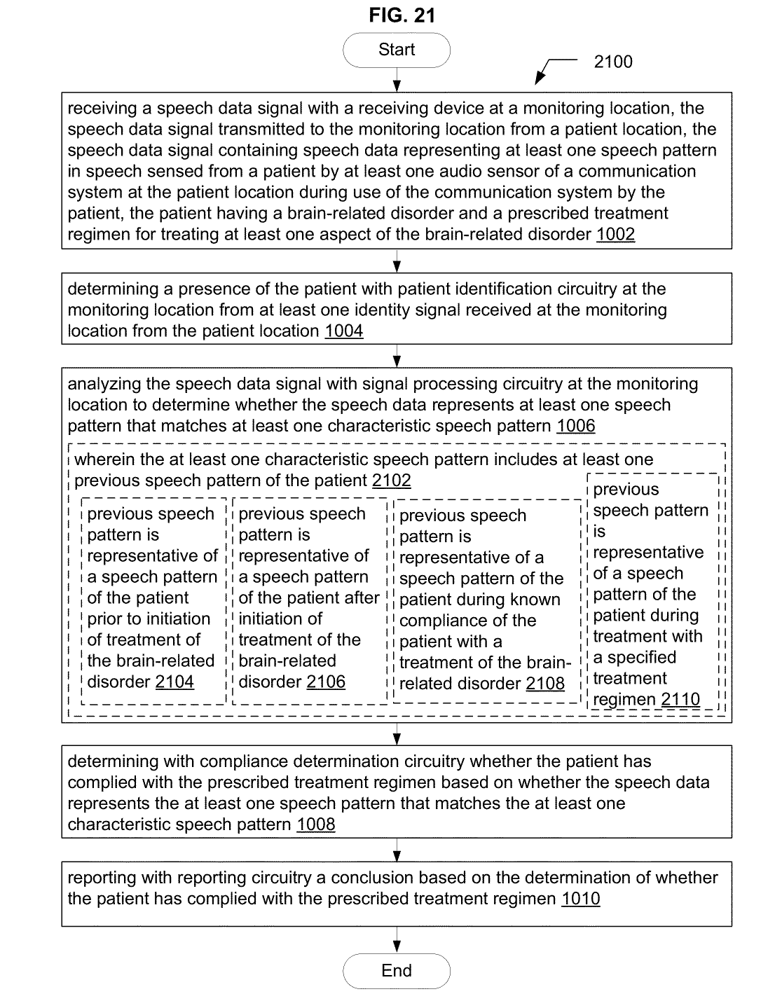

FIG. 21 depicts a method 2100 which includes steps 1002-1010 as described herein above. In an aspect of method 2100, the at least one characteristic speech pattern includes at least one previous speech pattern of the patient, as indicated at 2102. In various aspects, the at least one previous speech pattern is representative of at least one of speech pattern of the patient prior to initiation of treatment of the brain-related disorder, as indicated at 2104; a speech pattern of the patient after initiation of treatment of the brain-related disorder, as indicated at 2106; a speech pattern of the patient during known compliance of the patient with a treatment of the brain-related disorder, as indicated at 2108; and a speech pattern of the patient during treatment with a specified treatment regimen, as indicated at 2110.

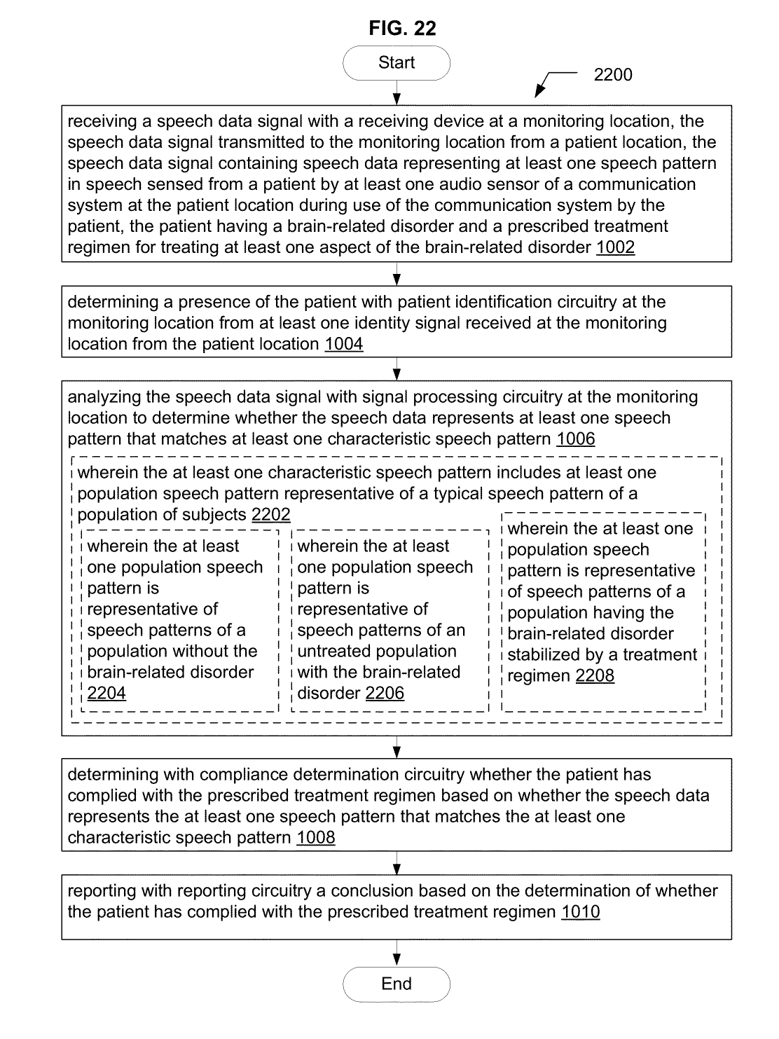

FIG. 22 depicts a method 2200 which includes steps 1002-1010 as described herein above. In an aspect of method 2200, the at least one characteristic speech pattern includes at least one population speech pattern representative of a typical speech pattern of a population of subjects, as indicated at 2202. In various aspects, the at least one population speech pattern is representative of at least one of speech patterns of a population without the brain-related disorder, as indicated at 2204; speech patterns of an untreated population with the brain-related disorder, as indicated at 2206; and speech patterns of a population having the brain-related disorder stabilized by a treatment regimen, as indicated at 2208.

In various embodiments, methods as described herein may be performed according to instructions implementable in hardware, software, and/or firmware. Such instructions may be stored in non-transitory machine-readable data storage media, for example. Those having skill in the art will recognize that the state of the art has progressed to the point where there is little distinction left between hardware, software, and/or firmware implementations of aspects of systems; the use of hardware, software, and/or firmware is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware in one or more machines, compositions of matter, and articles of manufacture. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically oriented hardware, software, and or firmware.

In some implementations described herein, logic and similar implementations may include software or other control structures. Electrical circuitry, for example, may have one or more paths of electrical current constructed and arranged to implement various functions as described herein. In some implementations, one or more media may be configured to bear a device-detectable implementation when such media hold or transmit device detectable instructions operable to perform as described herein. In some variants, for example, implementations may include an update or modification of existing software or firmware, or of gate arrays or programmable hardware, such as by performing a reception of or a transmission of one or more instructions in relation to one or more operations described herein. Alternatively or additionally, in some variants, an implementation may include special-purpose hardware, software, firmware components, and/or general-purpose components executing or otherwise invoking special-purpose components.

Implementations may include executing a special-purpose instruction sequence or invoking circuitry for enabling, triggering, coordinating, requesting, or otherwise causing one or more occurrences of virtually any functional operations described herein. In some variants, operational or other logical descriptions herein may be expressed as source code and compiled or otherwise invoked as an executable instruction sequence. In some contexts, for example, implementations may be provided, in whole or in part, by source code, such as C++, or other code sequences. In other implementations, source or other code implementation, using commercially available and/or techniques in the art, may be compiled/implemented/translated/converted into a high-level descriptor language (e.g., initially implementing described technologies in C or C++ programming language and thereafter converting the programming language implementation into a logic-synthesizable language implementation, a hardware description language implementation, a hardware design simulation implementation, and/or other such similar mode(s) of expression). For example, some or all of a logical expression (e.g., computer programming language implementation) may be manifested as a Verilog-type hardware description (e.g., via Hardware Description Language (HDL) and/or Very High Speed Integrated Circuit Hardware Descriptor Language (VHDL)) or other circuitry model which may then be used to create a physical implementation having hardware (e.g., an Application Specific Integrated Circuit). Those skilled in the art will recognize how to obtain, configure, and optimize suitable transmission or computational elements, material supplies, actuators, or other structures in light of these teachings.

This detailed description sets forth various embodiments of devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In an embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one having skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to non-transitory machine-readable data storage media such as a recordable type medium such as a floppy disk, a hard disk drive, a Compact Disc (CD), a Digital Video Disk (DVD), a digital tape, a computer memory, etc. A signal bearing medium may also include transmission type medium such as a digital and/or an analog communication medium (e.g., a fiber optic cable, a waveguide, a wired communications link, a wireless communication link (e.g., transmitter, receiver, transmission logic, reception logic, etc.) and so forth).

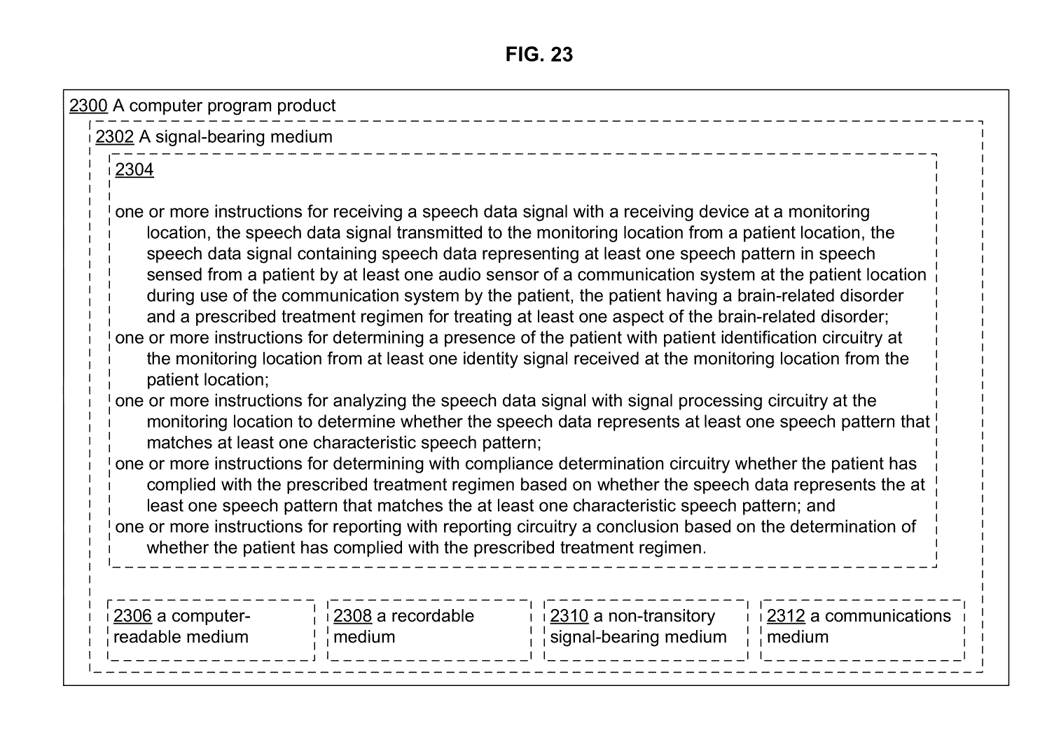

FIG. 23 is a block diagram of a computer program product 2300 for implementing a method as described in connection with FIG. 9. Computer program product 2300 includes a signal-bearing medium 2302 bearing one or more instructions for receiving a speech data signal with a receiving device at a monitoring location, the speech data signal transmitted to the monitoring location from a patient location, the speech data signal containing speech data representing at least one speech pattern in speech sensed from a patient by at least one audio sensor of a communication system at the patient location during use of the communication system by the patient, the patient having a brain-related disorder and a prescribed treatment regimen for treating at least one aspect of the brain-related disorder; one or more instructions for determining a presence of the patient with patient identification circuitry at the monitoring location from at least one identity signal received at the monitoring location from the patient location; one or more instructions for analyzing the speech data signal with signal processing circuitry at the monitoring location to determine whether the speech data represents at least one speech pattern that matches at least one characteristic speech pattern; one or more instructions for determining with compliance determination circuitry whether the patient has complied with the prescribed treatment regimen based on whether the speech data represents the at least one speech pattern that matches the at least one characteristic speech pattern; and one or more instructions for reporting with reporting circuitry a conclusion based on the determination of whether the patient has complied with the prescribed treatment regimen, as indicated at 2304. Signal-bearing medium 2302 may be, for example, a computer-readable medium 2306, a recordable medium 2308, a non-transitory signal-bearing medium 2310, or a communications medium 2312, examples of which are described herein above.

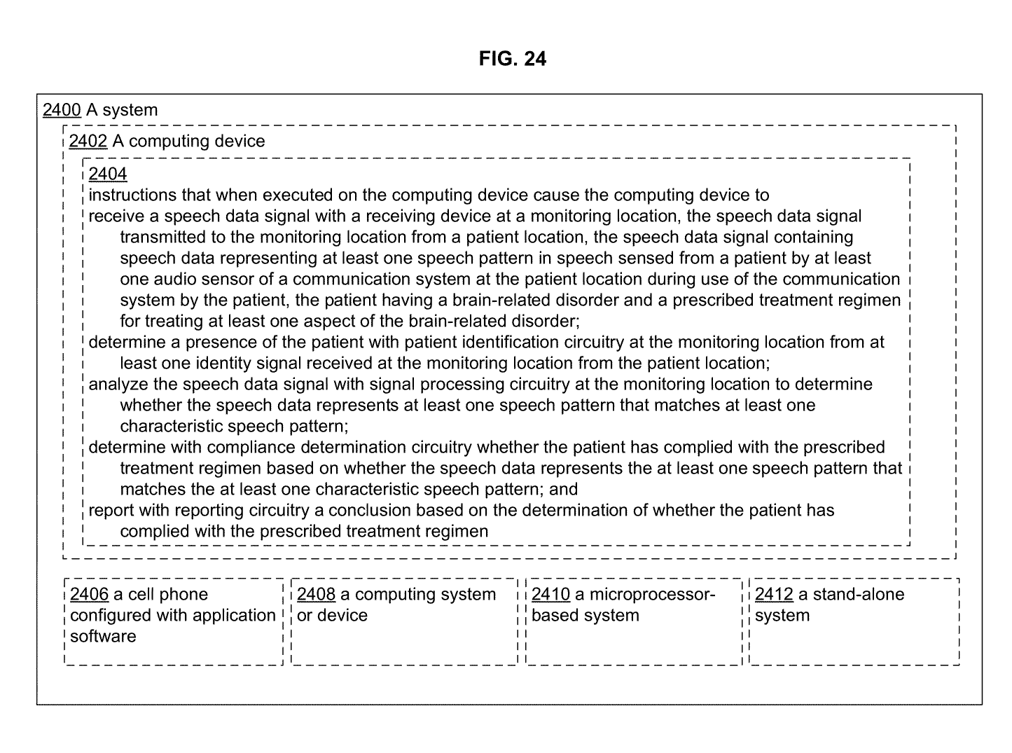

FIG. 24 is a block diagram of a system 2400 for implementing a method as described in connection with FIG. 9. System 2400 includes a computing device 2402 and instructions that when executed on the computing device cause the computing device to receive a speech data signal with a receiving device at a monitoring location, the speech data signal transmitted to the monitoring location from a patient location, the speech data signal containing speech data representing at least one speech pattern in speech sensed from a patient by at least one audio sensor of a communication system at the patient location during use of the communication system by the patient, the patient having a brain-related disorder and a prescribed treatment regimen for treating at least one aspect of the brain-related disorder; determine a presence of the patient with patient identification circuitry at the monitoring location from at least one identity signal received at the monitoring location from the patient location; analyze the speech data signal with signal processing circuitry at the monitoring location to determine whether the speech data represents at least one speech pattern that matches at least one characteristic speech pattern; determine with compliance determination circuitry whether the patient has complied with the prescribed treatment regimen based on whether the speech data represents the at least one speech pattern that matches the at least one characteristic speech pattern; and report with reporting circuitry a conclusion based on the determination of whether the patient has complied with the prescribed treatment regimen, as indicated at 2404. System 2400 may be, for example, a cell phone configured with application software 2406, a computing system or device 2408, a microprocessor-based system 2410, and/or a stand-alone system 2412.

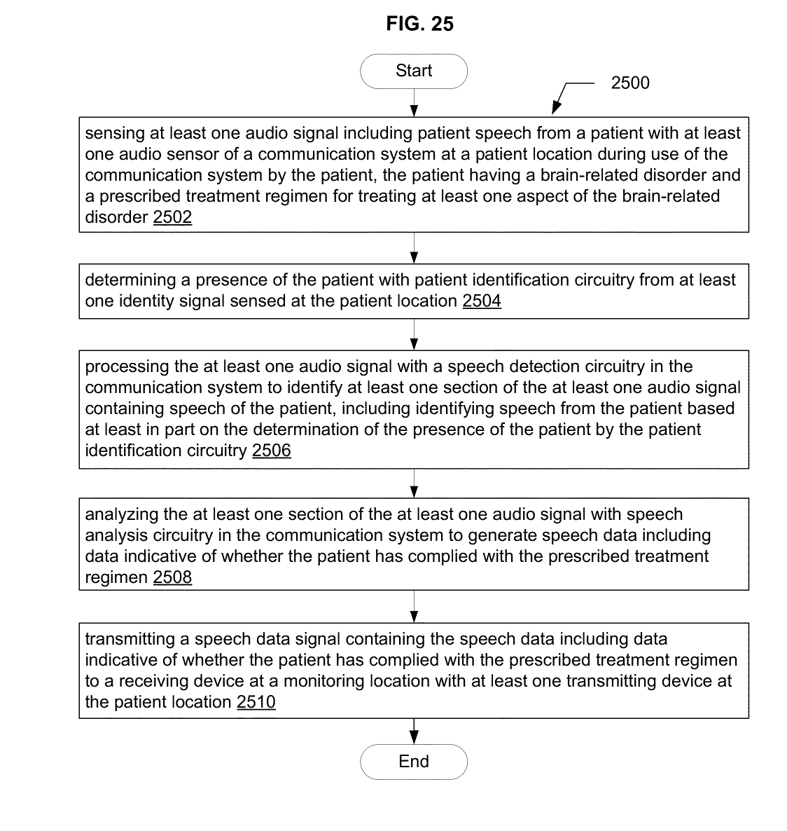

FIG. 25 is a flow diagram of a method 2500 relating to monitoring compliance of a patient with a prescribed treatment regimen. Method 2500 includes sensing at least one audio signal including patient speech from a patient with at least one audio sensor of a communication system at a patient location during use of the communication system by the patient, the patient having a brain-related disorder and a prescribed treatment regimen for treating at least one aspect of the brain-related disorder, as indicated at 2502; determining a presence of the patient with patient identification circuitry from at least one identity signal sensed at the patient location, as indicated at 2504; processing the at least one audio signal with speech detection circuitry in the communication system to identify at least one section of the at least one audio signal containing speech of the patient, including identifying speech from the patient based at least in part on the determination of the presence of the patient by the patient identification circuitry, as indicated at 2506; analyzing the at least one section of the at least one audio signal with speech analysis circuitry in the communication system to generate speech data including data indicative of whether the patient has complied with the prescribed treatment regimen, as indicated at 2508; and transmitting a speech data signal containing the speech data including data indicative of whether the patient has complied with the prescribed treatment regimen to a receiving device at a monitoring location with at least one transmitting device at the patient location, as indicated at 2510.

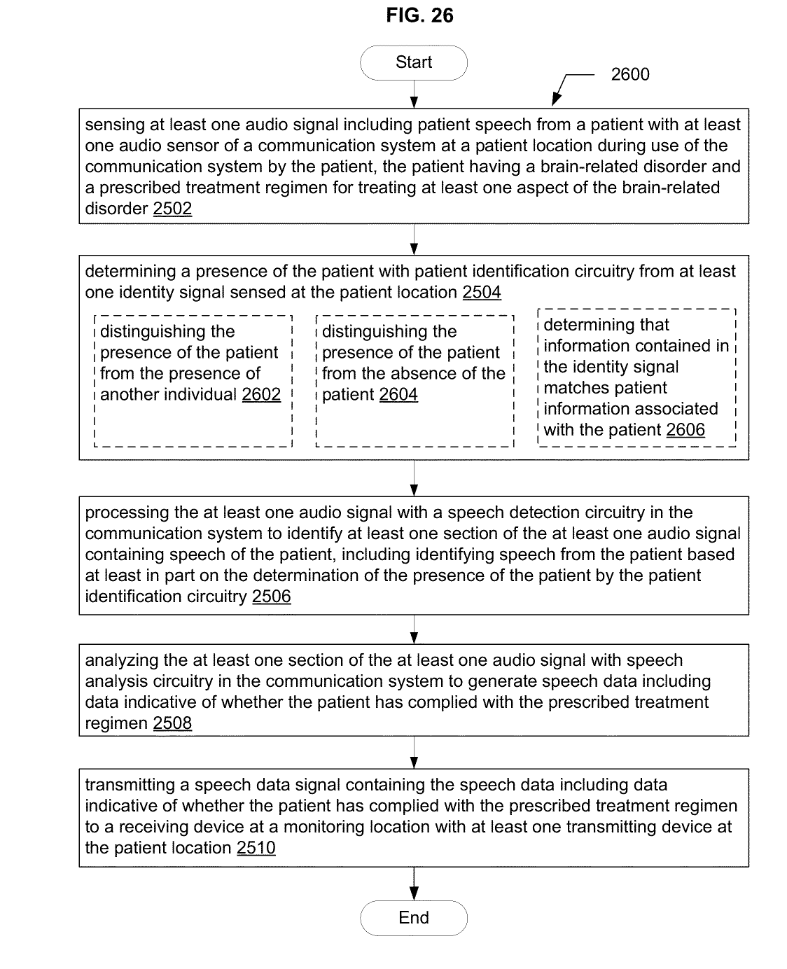

FIGS. 26-33 depict variations and expansions of method 2500 as shown in FIG. 25. In the methods depicted in FIGS. 26-33, steps 2502-2510 are as described generally in connection with FIG. 25. Here and elsewhere, method steps outlined with dashed lines represent steps that are included in some, but not all method aspects, and combinations of steps other than those specifically depicted in the figures are possible as would be known by those having ordinary skill in the relevant art.

FIG. 26 depicts a method 2600, including steps 2502-2510 as described above. In various aspects of method 2600, determining the presence of the patient includes at least one of distinguishing the presence of the patient from the presence of another individual, as indicated at 2602; distinguishing the presence of the patient from the absence of the patient, as indicated at 2604; and determining that information contained in the identity signal matches patient information associated with the patient, as indicated at 2606.

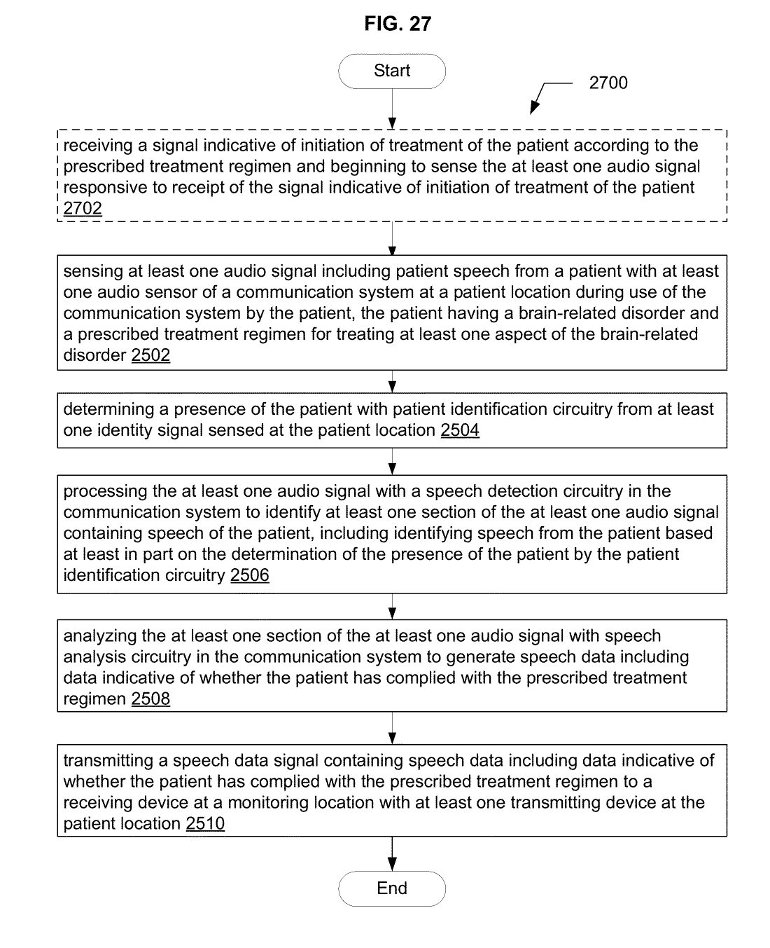

FIG. 27 depicts a method 2700, which includes receiving a signal indicative of initiation of treatment of the patient according to the prescribed treatment regimen and beginning to sense the at least one audio signal responsive to receipt of the signal indicative of initiation of treatment of the patient, as indicated at 2702.

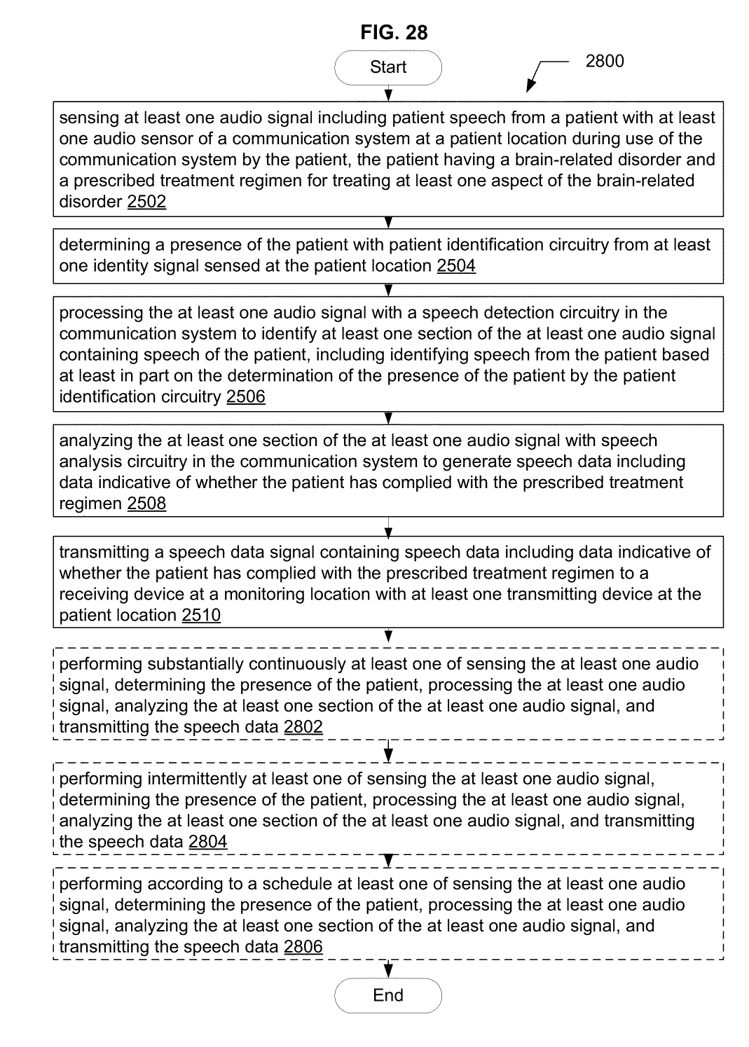

As shown in FIG. 28, in various aspects, a method 2800 includes performing substantially continuously at least one of sensing the at least one audio signal, determining the presence of the patient, processing the at least one audio signal, analyzing the at least one section of the at least one audio signal, and transmitting the speech data signal, as indicated at 2802. In another aspect, method 2800 includes performing intermittently at least one of sensing the at least one audio signal, determining the presence of the patient, processing the at least one audio signal, analyzing the at least one section of the at least one audio signal, and transmitting the speech data signal, as indicated at 2804. In another aspect, method 2800 includes performing according to a schedule at least one of sensing the at least one audio signal, determining the presence of the patient, processing the at least one audio signal, analyzing the at least one section of the at least one audio signal, and transmitting the speech data signal, as indicated at 2806.

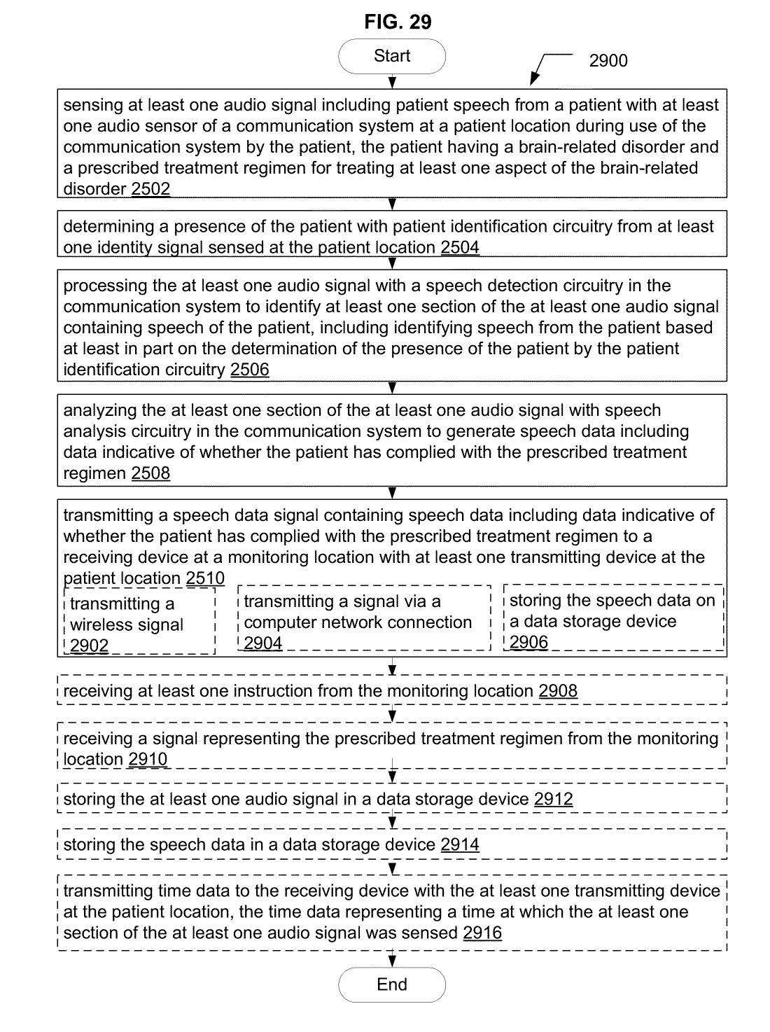

As shown in FIG. 29, in some aspects of method 2900, transmitting the speech data signal to the receiving device at the monitoring location includes at least one of transmitting a wireless signal, as indicated at 2902; transmitting a signal via a computer network connection, as indicated at 2904; and storing the speech data on a data storage device, as indicated at 2906. A data storage device may be, for example, a flash drive or a removable hard drive. In some aspects, method 2900 includes at least one of receiving at least one instruction from the monitoring location, as indicated at 2908; receiving a signal representing the prescribed treatment regimen from the monitoring location, as indicated at 2910; storing the at least one audio signal in a data storage device, as indicated at 2912; storing the speech data in a data storage device, as indicated at 2914; and transmitting time data to the receiving device with the at least one transmitting device at the patient location, the time data representing a time at which the at least one section of the at least one audio signal was sensed, as indicated at 2916.

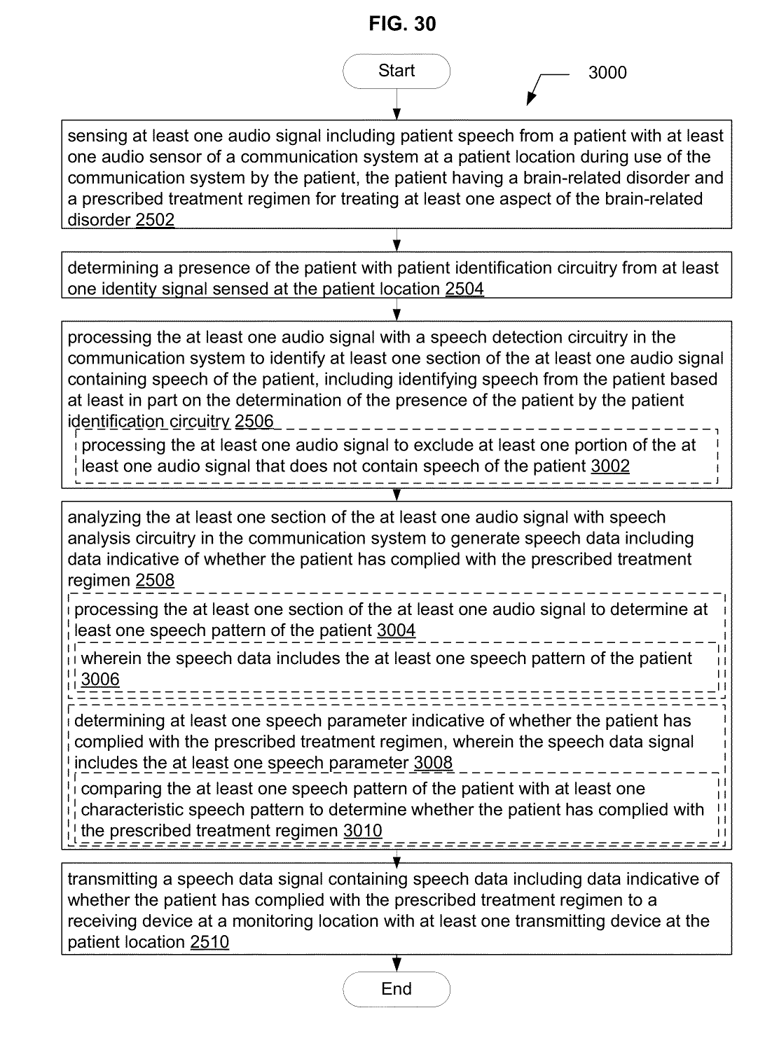

FIG. 30 depicts a method 3000. In an aspect, method 3000 includes processing the at least one audio signal to exclude at least one portion of the at least one audio signal that does not contain speech of the patient, as indicated at 3002. In another aspect, method 3000 includes processing the at least one section of the at least one audio signal to determine at least one speech pattern of the patient, as indicated at 3004. In an aspect, the speech data includes the at least one speech pattern of the patient, as indicated at 3006. In an aspect, method 3000 includes determining at least one speech parameter indicative of whether the patient has complied with the prescribed treatment regimen. In an aspect, the speech data includes the at least one speech parameter, as indicated at 3008. In addition, in an aspect, method 3000 includes comparing the at least one speech pattern of the patient with at least one characteristic speech pattern to determine whether the patient has complied with the prescribed treatment regimen, as indicated at 3010.

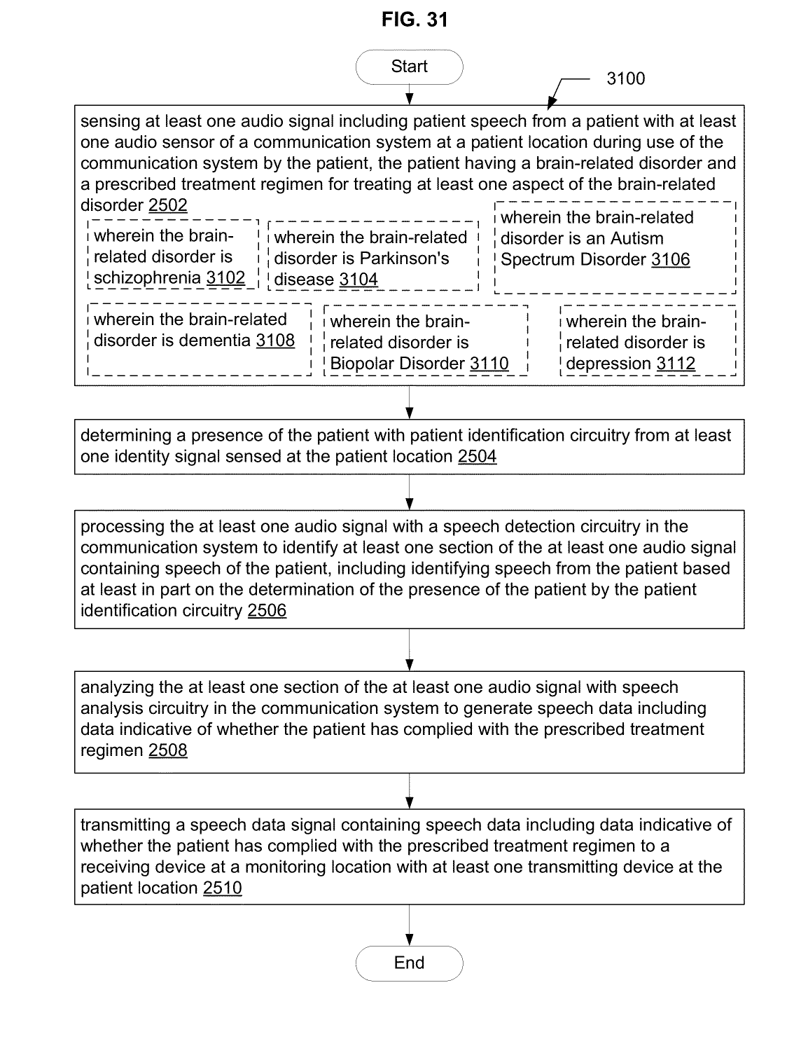

As shown in FIG. 31, in various aspects of a method 3100, the brain-related disorder includes at least one of schizophrenia, as indicated at 3102; Parkinson's disease, as indicated at 3104; an Autism Spectrum Disorder, as indicated at 3106; dementia, as indicated at 3108; Bipolar Disorder, as indicated at 3110; and depression, as indicated at 3112.

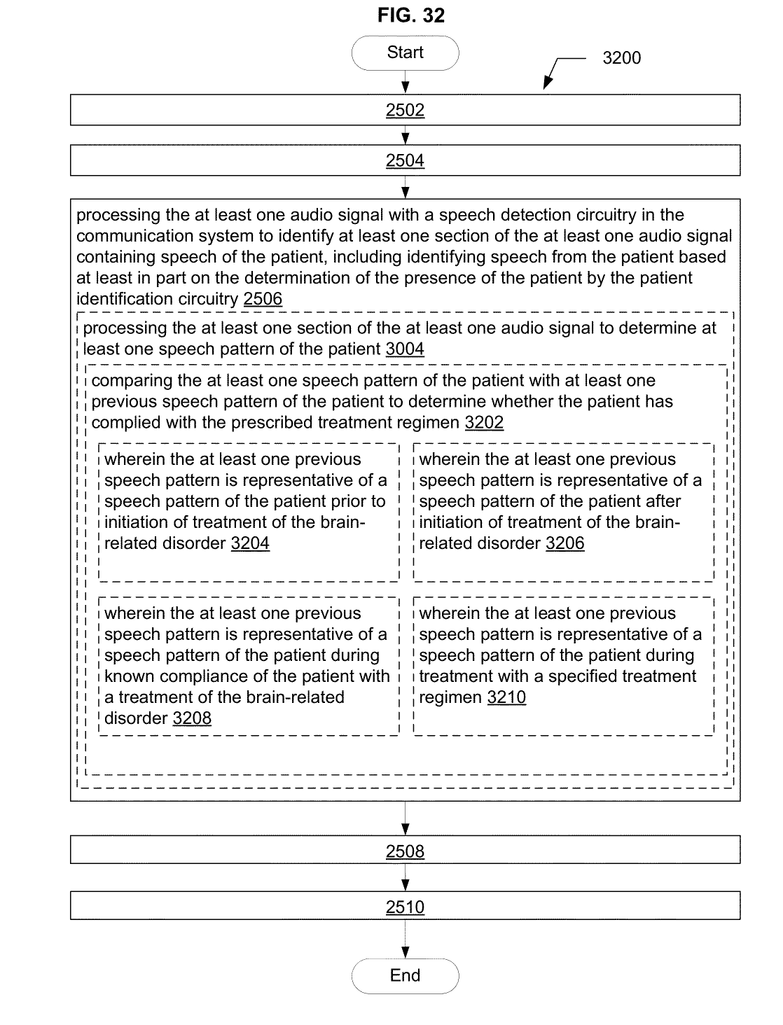

FIG. 32 depicts a method 3200, which includes processing the at least one section of the at least one audio signal to determine at least one speech pattern of the patient, as indicated at 3004, and then comparing the at least one speech pattern of the patient with at least one previous speech pattern of the patient to determine whether the patient has complied with the prescribed treatment regimen, as indicated at 3202. For example, in various aspects, the at least one previous speech pattern is representative of at least one of a speech pattern of the patient prior to initiation of treatment of the brain-related disorder, as indicated at 3204; a speech pattern of the patient after initiation of treatment of the brain-related disorder, as indicated at 3206; a speech pattern of the patient during known compliance of the patient with a treatment of the brain-related disorder, as indicated at 3208; and a speech pattern of the patient during treatment with a specified treatment regimen, as indicated at 3210.

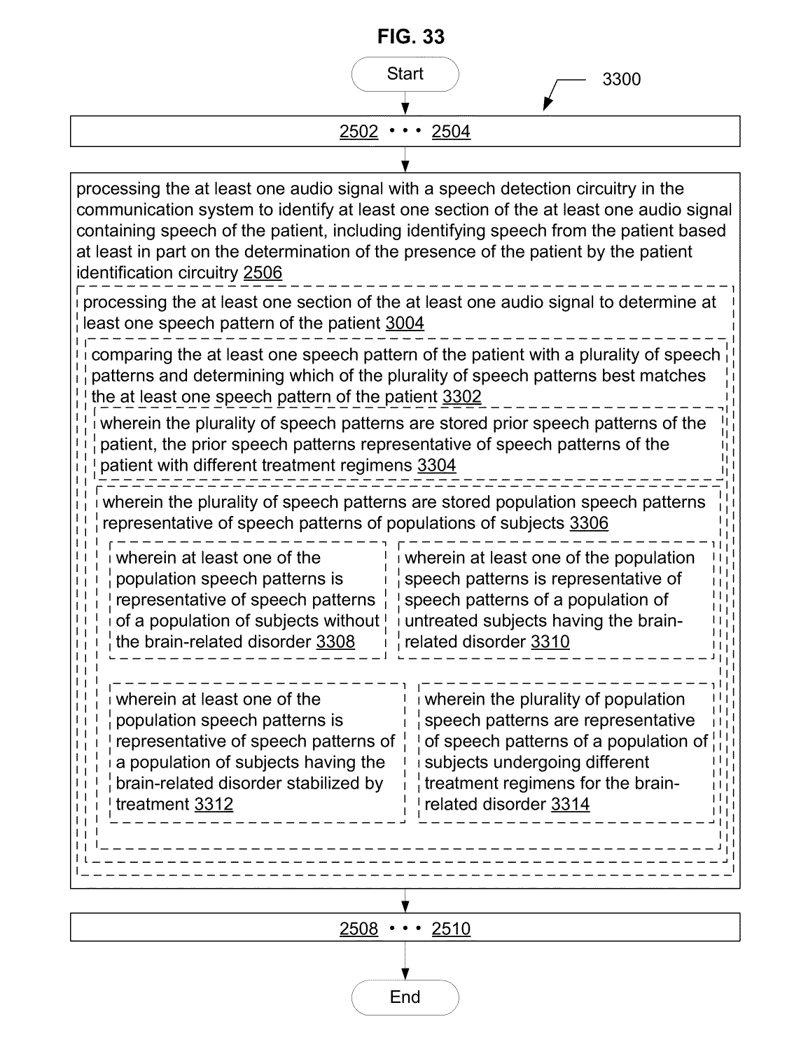

FIG. 33 depicts a method 3300, which includes processing the at least one section of the at least one audio signal to determine at least one speech pattern of the patient, as indicated at 3004, and then comparing the at least one speech pattern of the patient with a plurality of speech patterns and determining which of the plurality of speech patterns best matches the at least one speech pattern of the patient, as indicated at 3302. In an aspect, the plurality of speech patterns are stored prior speech patterns of the patient, the prior speech patterns representative of speech patterns of the patient with different treatment regimens, as indicated at 3304. In another aspect, the plurality of speech patterns are stored population speech patterns representative of speech patterns of populations of subjects, as indicated at 3306. For example, in various aspects, at least one of the population speech patterns is representative of speech patterns of a population of subjects without the brain-related disorder, as indicated at 3308; at least one of the population speech patterns is representative of speech patterns of a population of untreated subjects having the brain-related disorder, as indicated at 3310; at least one of the population speech patterns is representative of speech patterns of a population of subjects having the brain-related disorder stabilized by treatment, as indicated at 3312; or the plurality of population speech patterns are representative of speech patterns of a population of subjects undergoing different treatment regimens for the brain-related disorder, as indicated at 3314.

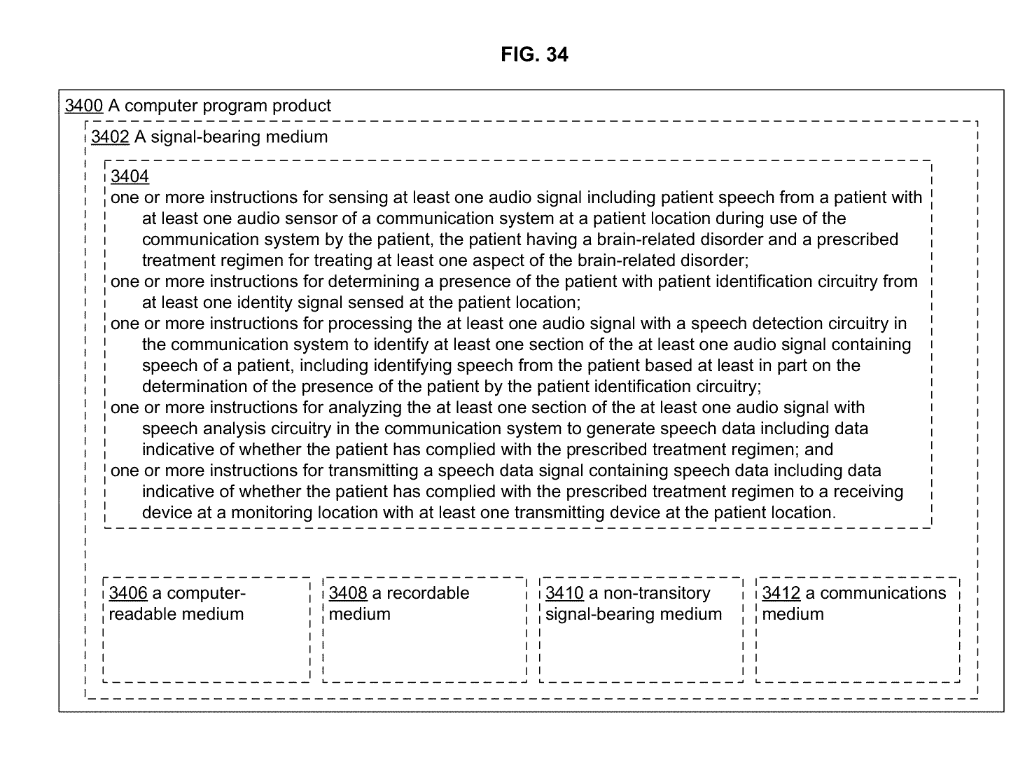

FIG. 34 is a block diagram of a computer program product 3400 for implementing a method as described in connection with FIG. 25. Computer program product 3400 includes a signal-bearing medium 3402 bearing one or more instructions for sensing at least one audio signal including patient speech from a patient with at least one audio sensor of a communication system at a patient location during use of the communication system by the patient, the patient having a brain-related disorder and a prescribed treatment regimen for treating at least one aspect of the brain-related disorder; one or more instructions for determining a presence of the patient with patient identification circuitry from at least one identity signal sensed at the patient location; one or more instructions for processing the at least one audio signal with a speech detection circuitry in the communication system to identify at least one section of the at least one audio signal containing speech of a patient, including identifying speech from the patient based at least in part on the determination of the presence of the patient by the patient identification circuitry; one or more instructions for analyzing the at least one section of the at least one audio signal with speech analysis circuitry in the communication system to generate speech data including data indicative of whether the patient has complied with the prescribed treatment regimen; and one or more instructions for transmitting a speech data signal containing the speech data including data indicative of whether the patient has complied with the prescribed treatment regimen to a receiving device at a monitoring location with at least one transmitting device at the patient location as indicated at 3404. Signal-bearing medium 3402 may be, for example, a computer-readable medium 3406, a recordable medium 3408, a non-transitory signal-bearing medium 3410, or a communications medium 3412, examples of which are described herein above.

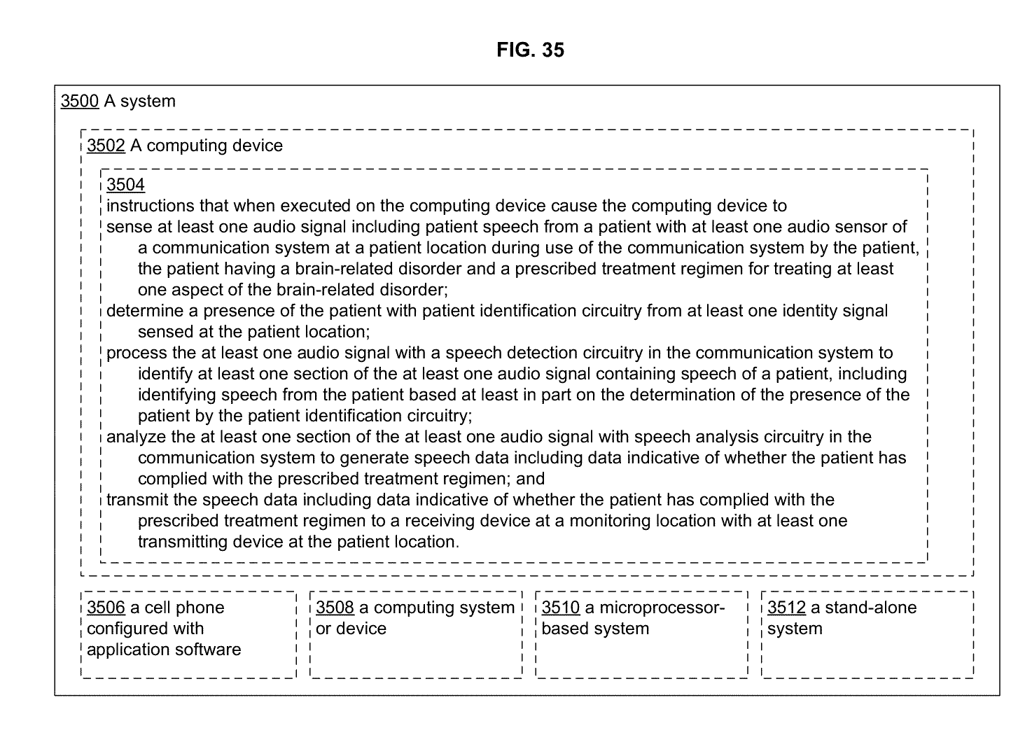

FIG. 35 is a block diagram of a system 3500 for implementing a method as described in connection with FIG. 25. System 3500 includes a computing device 3502 and instructions that when executed on the computing device cause the computing device to sense at least one audio signal including patient speech from a patient with at least one audio sensor of a communication system at a patient location during use of the communication system by the patient, the patient having a brain-related disorder and a prescribed treatment regimen for treating at least one aspect of the brain-related disorder; determine a presence of the patient with patient identification circuitry from at least one identity signal sensed at the patient location; process the at least one audio signal with a speech detection circuitry in the communication system to identify at least one section of the at least one audio signal containing speech of a patient, including identifying speech from the patient based at least in part on the determination of the presence of the patient by the patient identification circuitry; analyze the at least one section of the at least one audio signal with speech analysis circuitry in the communication system to generate speech data including data indicative of whether the patient has complied with the prescribed treatment regimen; and transmit the speech data including data indicative of whether the patient has complied with the prescribed treatment regimen to a receiving device at a monitoring location with at least one transmitting device at the patient location, as indicated at 3504. System 3500 may be, for example, a cell phone configured with application software 3506, a computing system or device 3508, a microprocessor-based system 3510, and/or a stand-alone system 3512.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

In some instances, one or more components may be referred to herein as “configured to,” “configured by,” “configurable to,” “operable/operative to,” “adapted/adaptable,” “able to,” “conformable/conformed to,” etc. Those skilled in the art will recognize that such terms (e.g., “configured to”) generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A communication system comprising:
at least one audio sensor for sensing at least one audio signal including patient speech during use of the communication system by a patient at a patient location, the patient having a brain-related disorder and a prescribed treatment regimen for treating at least one aspect of the brain-related disorder;
patient identification circuitry configured to determine a presence of the patient from at least one identity signal sensed at the patient location;
speech detection circuitry in the communication system for processing the at least one audio signal to identify at least one section of the at least one audio signal containing speech of the patient, wherein the speech detection circuitry is configured to identify the at least one section of the at least one audio signal containing speech of the patient based at least in part on the determination of the presence of the patient by the patient identification circuitry;
speech analysis circuitry in the communication system for analyzing the at least one section of the at least one audio signal to generate speech data including data indicative of whether the patient has complied with the prescribed treatment regimen; and
at least one transmitting device at the patient location for transmitting a speech data signal containing the speech data including data indicative of whether the patient has complied with the prescribed treatment regimen from the patient location to a receiving device at a monitoring location.

2. The system of claim 1, wherein the at least one identity signal includes at least a portion of the at least one audio signal, wherein the patient identification circuitry is configured to analyze the at least one audio signal to determine the presence of the patient by identifying at least a portion of the at least one audio signal that resembles known speech of the patient, and wherein the speech detection circuitry is configured to identify the at least one section of the at least one audio signal containing speech of the patient by identifying speech in the at least one audio signal corresponding to presence of the patient detected from the at least one audio signal.

3.-23. (canceled)

24. The system of claim 1, including a data storage device.

25. (canceled)

26. The system of claim 1, wherein the speech analysis circuitry includes a speech processor for processing at least one section of the at least one audio signal to determine at least one speech pattern of the patient.

27. (canceled)

28. The system of claim 26, wherein the speech processor includes a speech analyzer for assessing the at least one speech pattern to determine at least one speech parameter indicative of whether the patient has complied with the prescribed treatment regimen, wherein the speech data includes the at least one speech parameter.

29. The system of claim 26, wherein the speech analysis circuitry includes a comparator for comparing the at least one speech pattern with at least one characteristic speech pattern to determine whether the patient has complied with the prescribed treatment regimen.

30.-36. (canceled)

37. The system of claim 1, including notification circuitry for generating a notification.

38.-40. (canceled)

41. A method comprising:
sensing at least one audio signal including patient speech from a patient with at least one audio sensor of a communication system at a patient location during use of the communication system by the patient, the patient having a brain-related disorder and a prescribed treatment regimen for treating at least one aspect of the brain-related disorder;
determining a presence of the patient with patient identification circuitry from at least one identity signal sensed at the patient location;
processing the at least one audio signal with a speech detection circuitry in the communication system to identify at least one section of the at least one audio signal containing speech of the patient, including identifying speech from the patient based at least in part on the determination of the presence of the patient by the patient identification circuitry;
analyzing the at least one section of the at least one audio signal with speech analysis circuitry in the communication system to generate speech data including data indicative of whether the patient has complied with the prescribed treatment regimen; and
transmitting a speech data signal containing the speech data including data indicative of whether the patient has complied with the prescribed treatment regimen to a receiving device at a monitoring location with at least one transmitting device at the patient location.

42.-44. (canceled)

45. The method of claim 41, including receiving a signal indicative of initiation of treatment of the patient according to the prescribed treatment regimen and beginning to sense the at least one audio signal responsive to receipt of the signal indicative of initiation of treatment of the patient.

46.-54. (canceled)

55. The method of claim 41, including transmitting time data to the receiving device with the at least one transmitting device at the patient location, the time data representing a time at which the at least one section of the at least one audio signal was sensed.

56. (canceled)

57. The method of claim 41, including processing the at least one section of the at least one audio signal to determine at least one speech pattern of the patient.

58. The method of claim 57, wherein the speech data includes the at least one speech pattern of the patient.

59. The method of claim 41, including determining at least one speech parameter indicative of whether the patient has complied with the prescribed treatment regimen, wherein the speech data includes the at least one speech parameter.

60. The method of claim 57, including comparing the at least one speech pattern of the patient with at least one characteristic speech pattern to determine whether the patient has complied with the prescribed treatment regimen.

61.-66. (canceled)

67. The method of claim 57, including comparing the at least one speech pattern of the patient with at least one previous speech pattern of the patient to determine whether the patient has complied with the prescribed treatment regimen.

68.-71. (canceled)

72. The method of claim 57, including comparing the at least one speech pattern of the patient with a plurality of speech patterns and determining which of the plurality of speech patterns best matches the at least one speech pattern of the patient.

73.-78. (canceled)

79. A computer program product comprising:
a non-transitory signal-bearing medium bearing:
one or more instructions for sensing at least one audio signal including patient speech from a patient with at least one audio sensor of a communication system at a patient location during use of the communication system by the patient, the patient having a brain-related disorder and a prescribed treatment regimen for treating at least one aspect of the brain-related disorder;
one or more instructions for determining a presence of the patient with patient identification circuitry from at least one identity signal sensed at the patient location;
one or more instructions for processing the at least one audio signal with a speech detection circuitry in the communication system to identify at least one section of the at least one audio signal containing speech of a patient, including identifying speech from the patient based at least in part on the determination of the presence of the patient by the patient identification circuitry;
one or more instructions for analyzing the at least one section of the at least one audio signal with speech analysis circuitry in the communication system to generate speech data including data indicative of whether the patient has complied with the prescribed treatment regimen; and
one or more instructions for transmitting a speech data signal containing the speech data including data indicative of whether the patient has complied with the prescribed treatment regimen to a receiving device at a monitoring location with at least one transmitting device at the patient location.

80.-82. (canceled)

83. The computer program product of claim 79, wherein the non-transitory signal-bearing medium bears one or more instructions for receiving a signal indicative of initiation of treatment of the patient according to the prescribed treatment regimen and beginning to sense the at least one audio signal responsive to receipt of the signal indicative of initiation of treatment of the patient.

84.-93. (canceled)

94. The computer program product of claim 79, wherein the non-transitory signal-bearing medium bears one or more instructions for transmitting time data to the receiving device with the at least one transmitting device at the patient location, the time data representing a time at which the at least one section of the at least one audio signal was sensed.

95. The computer program product of claim 79, wherein the non-transitory signal-bearing medium bears one or more instructions for processing the at least one audio signal to exclude at least one portion of the at least one audio signal that does not contain speech of the patient.

96. The computer program product of claim 79, wherein the non-transitory signal-bearing medium bears one or more instructions for processing the at least one section of the at least one audio signal to determine at least one speech pattern of the patient.

97. The computer program product of claim 96, wherein the one or more instructions for analyzing the at least one section of the at least one audio signal with speech analysis circuitry in the communication system to generate speech data including data indicative of whether the patient has complied with the prescribed treatment regimen include the one or more instructions for processing the at least one section of the at least one audio signal to determine the at least one speech pattern of the patient.

98. The computer program product of claim 79, wherein the non-transitory signal-bearing medium bears one or more instructions for determining at least one speech parameter indicative of whether the patient has complied with the prescribed treatment regimen, wherein the speech data includes the at least one speech parameter.

99.-104. (canceled)

105. The computer program product of claim 96, wherein the non-transitory signal bearing medium bears one or more instructions for comparing the at least one speech pattern of the patient with a plurality of speech patterns and determining which of the plurality of speech patterns best matches the at least one speech pattern of the patient.

106.-108. (canceled)

109. The system of claim 1, further including at least one sensor, the at least one sensor including at least one of a biometric sensor, an audio sensor, an imaging device, an RFID sensor, and a user input device.

110. The system of claim 1, wherein the at least one identity signal includes at least one of an authentication factor, a cell phone identification code, and an RFID signal.

111. The system of claim 1, wherein the patient identification circuitry is configured to determine the presence of the patient by at least one of distinguishing the presence of the patient from the presence of another individual, distinguishing the presence of the patient from the absence of the patient, and determining that information contained in the identity signal matches patient information associated with the patient.

112. The system of claim 1, wherein the at least one audio sensor, the patient identification circuitry, the speech detection circuitry, the speech analysis circuitry, and the at least one transmitting device are components of at least one of a cell phone configured with application software, a computing system or device, an intercommunication system, and a microprocessor-based system.

113. The system of claim 1, wherein the transmitting device includes at least one of a wireless transmitter, a computer network connection, and a computer communication port.

114. The system of claim 37, wherein the notification circuitry includes at least one of circuitry for generating an email notification, circuitry for generating a notification to be transmitted to a wireless device, and circuitry for storing a notification in a data storage device.

115. The method of claim 41, wherein determining the presence of the patient includes at least one of distinguishing the presence of the patient from the presence of another individual, distinguishing the presence of the patient from the absence of the patient, and determining that information contained in the identity signal matches patient information associated with the patient.

116. The method of claim 41, including performing at least one of sensing the at least one audio signal, determining the presence of the patient, processing the at least one audio signal, analyzing the at least one section of the at least one audio signal, and transmitting the speech data signal; wherein the performing is done substantially continuously, intermittently, or according to a schedule.

117. The method of claim 41, including receiving at least one of at least one instruction and a signal representing the prescribed treatment regimen from the monitoring location.

118. The method of claim 41, wherein transmitting the speech data signal to the receiving device at the monitoring location includes at least one of transmitting a wireless signal and transmitting a signal via a computer network connection.

119. The method of claim 41, including at least one of storing the at least one audio signal in a data storage device and storing the speech data in a data storage device.

120. The method of claim 41, wherein the brain-related disorder includes at least one of schizophrenia, Parkinson's disease, Autism Spectrum Disorder, dementia, Bipolar Disorder, and depression.

121. The method of claim 67, wherein the at least one previous speech pattern is representative of at least one of a speech pattern of the patient prior to initiation of treatment of the brain-related disorder, a speech pattern of the patient after initiation of treatment of the brain-related disorder, a speech pattern of the patient during known compliance of the patient with a treatment of the brain-related disorder, and a speech pattern of the patient during treatment with a specified treatment regimen.

122. The method of claim 72, wherein the plurality of speech patterns are selected from stored population speech patterns representative of speech patterns of populations of subjects and stored prior speech patterns of the patient representative of speech patterns of the patient with different treatment regimens.

123. The method of claim 122, wherein the plurality of speech patterns are the stored population speech patterns, and wherein at least one of the population speech patterns is representative of at least one of speech patterns of a population of subjects without the brain-related disorder, speech patterns of a population of untreated subjects having the brain-related disorder, speech patterns of a population of subjects having the brain-related disorder stabilized by treatment, and speech patterns of a population of subjects undergoing different treatment regimens for the brain-related disorder.

124. The computer program product of claim 79, wherein the one or more instructions for determining the presence of the patient include one or more instructions for at least one of distinguishing the presence of the patient from the presence of another individual, distinguishing the presence of the patient from the absence of the patient, and determining that information contained in the identity signal matches patient information associated with the patient.

125. The computer program product of claim 79, wherein the non-transitory signal-bearing medium bears one or more instructions for performing, substantially continuously, intermittently, or according to a schedule, at least one of sensing the at least one audio signal, determining the presence of the patient, processing the at least one audio signal, analyzing the at least one section of the at least one audio signal, and transmitting the speech data signal.

126. The computer program product of claim 79, wherein the non-transitory signal-bearing medium bears one or more instructions for at least one of receiving at least one instruction from the monitoring location and receiving a signal representing the prescribed treatment regimen from the monitoring location.

127. The computer program product of claim 96, wherein the non-transitory signal-bearing medium bears one or more instructions for at least one of comparing the at least one speech pattern of the patient with at least one characteristic speech pattern to determine whether the patient has complied with the prescribed treatment regimen and comparing the at least one speech pattern of the patient with at least one previous speech pattern of the patient to determine whether the patient has complied with the prescribed treatment regimen.
